(19) United States
(12) Patent Application Publication
(10) Pub. No.: US 2026/0159842 A1
(43) Pub. Date: Jun. 11, 2026

(54) VIRAL VECTOR MEDIATED GENE THERAPY TRANSDUCTION AND SAFETY OR PROMOTING VIRAL CLEARANCE AND/OR INHIBITING VIRAL INFECTIVITY BY TARGETING CLASS B SCAVENGER RECEPTORS

(71) Applicant: The United States of America, as represented by the Secretary, Dept. of Health & Human Services, Bethesda, MD (US)

(72) Inventors: Alexander V. Bocharov, Bethesda, MD (US); Thomas L. Eggerman, Rockville, MD (US); Amy P. Patterson, Rockville, MD (US); Irina N. Baranova, Silver Spring, MD (US); Tatyana G. Vishnyakova, Silver Spring, MD (US); Zhigang Chen, Gaithersburg, MD (US)

(73) Assignee: The United States of America, as represented by the Secretary, Dept. of Health & Human Services, Bethesda, MD (US)

(21) Appl. No.: 19/303,076

(22) Filed: Aug. 18, 2025

Related U.S. Application Data
(60) Provisional application No. 63/684,214, filed on Aug. 16, 2024.

(51) Int. Cl. C12N 15/113; A61K 38/00; A61P 31/14; C07K 14/00; C07K 16/28; C12N 15/86

(52) U.S. Cl. CPC C12N 15/1138; A61P 31/14; C07K 14/001; C07K 16/28; C12N 15/86; A61K 38/00; C07K 2319/70; C12N 2310/14; C12N 2310/531; C12N 2710/10043; C12N 2740/15043; C12N 2750/14143

(57) ABSTRACT

In one embodiment, the invention provides a method of promoting viral vector-mediated gene transfer to an animal or to cells or tissues ex vivo. In another embodiment, the invention provides a method of promoting viral clearance from an animal, the method comprising administering to an animal having or at risk of a viral infection an active principal that up-regulates the expression/activity of one or more SR-Bs involved in viral clearance. In one embodiment, the invention provides a method of inhibiting viral infectivity by utilizing SR-B antagonists, antibodies, or shRNA to prevent or reduce viral entry or reduce virally induced cell fusion or interfere with intracellular viral processing in virally targeted cells.

### CROSS-REFERENCE TO RELATED APPLICATIONS

This patent application claims the benefit of U.S. Provisional Patent Application No. 63/684,214, filed Aug. 16, 2024, which is incorporated by reference.

### STATEMENT REGARDING FEDERALLY SPONSORED RESEARCH OR DEVELOPMENT

This invention was made with Government support under National Institutes of Health project numbers ZIA CL090096 by the National Institute of Diabetes and Digestive and Kidney Diseases and ZIA and CL090097 by the National Institute of Allergy and Infectious Diseases. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 89,571 Byte Extensible Markup Language (XML) file named “774347_ST26.xml,” Aug. 18, 2025.

### BACKGROUND OF THE INVENTION

Gene therapy is a molecular therapeutic targeting approach, especially for genetic disease that has been a developing field for 30 years. The technology has transformed many areas of medicine, including therapeutic gene transfer, vaccine development, and experimental medical research. An important technology underlying many gene therapy approaches is viral vector-mediated gene transfer technology, in which a modified virus (containing a transgene to impart a physiological change in the recipient animal) is introduced into the recipient animal (e.g., an animal subject or a human or animal patient) and used as a medicament. The treatment can be given into the systemic circulation, for example, intravenously. Alternatively, it can be given as a direct injection into a tissue/body space, for example into the eye or it can be used ex vivo where a subject's cells of interest are collected and the viral vector is directly exposed to the cells and the treated cells are given back to the subject

Despite distinct advantages of viral vector-based technologies, the efficiency of gene delivery when introduced into the systemic circulation is often compromised due to rapid removal of viral vectors by the reticuloendothelial system (RES), especially by liver sinusoidal endothelial cells (LSEC) and liver Kupffer cells (KCs). Additionally, viral vector administration also is associated with undesirable effects, such as innate immune system activation, cellular toxicity, and thrombocytopenia. Sequestration of viral particles (such as adenoviral “AdV” vectors) by the liver RES is a host antiviral defense mechanism including adenoviral clearance, which not only strongly decreases the ability of viral vectors to reach target tissues, but also provokes toxic responses and triggers a rapid destruction of KCs, among other responses.

This host defense mechanism poses technical challenges for employing viral vector-based gene transfer technology in a clinical setting. Rapid clearing of viral vectors from a host animal (e.g., a patient) can often lead to complications (i.e., side-effects) such as adverse events, low transduction efficiency, off target effects, and antibody induction; complications requiring a need to administer viral vectors at high titer to achieve a given level of transduction efficiency in the face of the host-defense response against such viral vectors. Such technical challenges have slowed development of viral vector-based technologies, as applied in the area of gene therapy. However, the present invention addresses these concerns.

### BRIEF SUMMARY OF THE INVENTION

The invention is predicated on the discovery (reported below in the Example) that class B scavenger receptor family proteins (SR-B), which family includes SRBI, SRBII, LIMP2 and CD36, act as an antiviral defense mechanism protecting parenchymal cells of various tissues from direct virus interaction and infection by clearing viruses in SR-Bs-expressing RES, especially liver sinusoidal endothelial cells.

Additionally, SR-B proteins act as receptors/co-receptors during viral infections, including epithelial cells as well as bone marrow derived phagocytes (example 3).

SR-BI is required for virus-induced cell fusion which is a primary mechanism of viral dissemination in HIV, HCV and COVID19 infections.

Building on this discovery, the invention provides a method for increasing viral vector-mediated gene transfer, which comprises reducing viral vector clearance by blocking or downregulating SR-Bs in liver sinusoidal endothelial cells (LSEC) or other cells within the reticulo-endothelial system (RES). The method can be achieved, for example, by administering to animals receiving viral vector-mediated gene transfer SR-B antagonist agents such as synthetic amphipathic helical peptides (SAHP), anti-SR-B antibodies, or small molecule SR-B antagonists, or the method can be achieved via SR-B expression down-regulation, such as through shRNA or small molecules, in such animals.

By reducing viral vector clearance by blocking or downregulating SR-B, the invention can permit higher transfection efficiencies in target tissues, thus reducing viral vector doses needed to achieve a given level of transduction efficiency, and consequently vector toxicity. In turn, reduced viral vector doses can provide greater safety, reduce production and administration costs, and make possible a greater number of gene therapy approaches in a larger number of target diseases.

Another approach to improve target cell response to viral vectors is to increase the transduction efficiency approaches include increasing cellular viral vector uptake by increasing the number or activity of viral vector receptors, improving intracellular vector processing or decreasing intracellular vector degradation. Up regulation of SR-B expression/activity in viral vector target cells can increase viral vector transduction. Also, the invention provides a method of promoting viral clearance from an animal, the method comprising administering to an animal having or at risk of a viral infection an active principal that up-regulates one or more SR-Bs as well as decreasing viral infectivity in virally targeted cells by down regulating one or more SR-Bs.

### BRIEF DESCRIPTION OF THE SEVERAL VIEWS OF THE DRAWING(S)

FIG. 1 presents data concerning CD36-mediated uptake of various ligands including adenovirus V5 (AdV5). HeLa cells cultured on 12-well plates were incubated with nothing added (A), 10 ug/ml of Alexa 488-labeled LDL (B), oxLDL (C), or 10⁷ pfu/ml of Cy2-labeled AdV5 (D) at 37° C. for 2 hours. Cells were washed with PBS three times and collected utilizing a cell stripper solution. Cell associated fluorescence was measured by FACS Analyzer. Mock-Hela (E) and CD36-HeLa (F and G) cells cultured on glass slides were incubated with 20 μg/ml Alexa 488-AdV5 (green) in DMEM, 2 mg/ml Fatty Acid Free BSA for 2 hours, and pulse-chased with fresh media containing 5 μg/ml of Alexa 568-Transferrin (panel E and G, red with overlayed DIC image) or Lysotracker Red (panel F, red) for another 30 minutes. Cells were washed with PBS and visualized utilizing confocal microscopy.

FIG. 2 presents transmitting electron microscope images showing AdV5 adhesion, endocytosis, cytoplasmic escape and nuclear accumulation in CD36 overexpressing HeLa cells. CD36-overexpressing HeLa cells were incubated with AdV5 (10⁸/mlPFU/ml) and processed as described in Materials and Methods. Arrows show cell surface bound AdV5 (panel A), endosomal (panel B), cytosolic (panel C) and nuclear (panel D) localization.

FIG. 3 presents data concerning AdV5 uptake assessed by Luciferase and GFP reporter expression in CD36 and wild type (WT) HeLa. HeLa cells with or without CD36 overexpression were incubated with various concentrations of LUC-AdV5 for two hours (Panel A) or with 10⁷ pfu/ml of LUC-AdV5 for various times (Panel B) or with 10⁷ pfu/ml of GFP-AdV5 for two hours (Panel C) at 37° C. Cells were then washed with PBS three times and further incubated in DMEM containing 10% FCS for another 24 hours. Cells then were washed with PBS three times, lysed and analyzed for LUC activity. Alternatively, cells were collected utilizing cell stripper solution and cell-associated fluorescence was measured by a FACS analyzer.

FIG. 4 presents data concerning CD36-dependent AdV5 induced cytotoxicity as measured by cell monolayer resistance (Panel A) and LDH release (Panel B). Mock-transfected and CD36 overexpressing HEK293 cells were plated at 75% confluency. 24 hours later, 10⁸ pfu/ml of AdV5 were added and further incubated for another 72 hours. Cell monolayer resistance was measured using ACEA RTCA (A). For assessing LDH release, cells were incubated for 24 hours in the presence of 10⁸ pfu/ml AdV5. Media were subsequently collected and LDH was measured in the media as indicated in Methods (Panel B).

FIG. 5 presents data concerning Alexa 488-HDL and AdV5 uptake in mouse liver (2 hours after IV/RO inoculation). WT (Panels A, C) and CD36KO (Panels B, D) animals were IV injected with 4×10⁶ PFU/g of Alexa 488-AdV5 (Panels A, B) or 30 μg/g of Alexa488-HDL (C, D). Two hours later, mice were euthanized, the livers were perfused to wash out unbound ligands for image analyses utilizing confocal microscopy. Liver tissues were also homogenized and extracted with 1% Triton X100 in 50 mM Tris, pH-7.4. Fluorescence in a centrifugation cleared extracts were counted utilizing a Victor 3 fluorimeter (Panel E).

FIG. 6 presents data concerning uptake of Alexa 488/568-AdV5 in NPLC and hepatocytes. NPLC and hepatocytes were isolated from WT mice and cultivated for 24 hours as described in Methods. NPLC (Panels A, B, C, F) and hepatocyte-NPLC co-cultures (Panels D, E) were incubated with Alexa488-AdV5, green, (Panel A, B, D, E) followed by a pulse-chase incubation with Alexa 568-transferrin (Panels A, D) or Lysotracker Red (Panels B, E), both red. In Panels C and F, NPLC were isolated from WT mice, IV inoculated with 4×10⁶ PFU/g of Alexa 568-AdV5, further cultivated for 6 hours, fixed with 3.6% paraformaldehyde and stained for CD36, green (Panel C) or for CD31, green (Panel F). Images were analyzed using confocal microscopy. Yellow represents co-localized merged red/green signals.

FIG. 7 presents data concerning AdV5 Luciferase transduction in hepatocyte, LSEC and KC culture from WT and CD36 KO rat. Cells isolated and plated as described in Materials and Methods were cultivated for 24 hours. Hepatocyte (panel A), LSEC (panel B) and KC (panels C) were incubated with various concentrations (10⁵-10⁸ pfu/ml) of luciferase expressing AdV5 particles. The cells were washed with PBS and further incubated in virus free media for 48 hours. Luciferase activity was measured in cell lysates.

FIG. 8 presents data concerning AdV5 transduction/infective capacity as estimated by GFP (green) expression in co-culture of NPLC and hepatocytes. NPLC and hepatocytes were isolated from WT (A, B) and CD36 KO (C, D, E) mice and cultivated for 24 hours as described in Methods. Hepatocyte-NPLC co-cultures were incubated with 10⁸ pfu/ml of AdV5-GFP (green). Panels A, C represent bright field microscopy of various cells and the detection of GFP signal is shown in panels B, D, E.

FIG. 9 presents data concerning AdV5 transduction/infective capacity as estimated by GFP (green) expression in organs of CD36KO and WT mice. CD36 KO (Panels A-C) or WT (Panels D-F) mice were IV injected with 4×10⁶ PFU/g of GFP-AdV5. Twenty-two hours later mice were further IV injected with 15 μg/ml of Alexa 568-HDL (red). Two additional hours later mice were euthanized and perfused through inferior vena cava to wash out unbound ligands. Liver (Panels A, D), lungs (Panels B, E) and kidneys (Panels C, F) were collected for confocal microscopy analyses.

FIG. 10 presents data concerning AdV5-GFP infectivity analyses by GFP and GFP RNA expression in organs of WT and CD36-KO mice 48 hours after AdV5-GFP IV injection. GFP fluorescence measurement (arbitrary units) of liver tissue lysates were prepared as described in Materials and Methods (panel A). Western blot analyses of GFP protein expression (upper panel) were performed in liver lysates from mice following IV AdV5-GFP injection (panel B), Control without AdV5 (N=1), WT (N=4) and CD36-KO (N=4). Corresponding levels of CD36 and 3-actin expression (used as the sample loading control) are shown in lower panel. GFP gene expression analyses in different organs by qRT-PCR (panel C). GFP expression levels were normalized by GAPDH and are presented as the fold changes relatively to “none” (corresponding non-treated controls). Individual data points are plotted along with mean values SD (n=4). *, P<0.05, ** P<0.01, CD36Ko-AdV5-treated versus WT-AdV5-treated mice.

FIG. 11 presents data concerning human CD36 (hCD36) expression in CD36KO and WT mice decreased AdV5-GFP hepatic infectivity. CD36 KO mice were IV injected with 4×10⁶ PFU/g of empty AdV5 (Panel A) or hCD36-AdV5 (Panel B). Twenty-four hours later, mice were given AdV5-GFP and then 22 hours later mice were additionally R/O injected with 15 μg/ml of Alexa 633-HDL. Two hours later mice were sacrificed, and liver were perfused through the inferior vena cava to wash out unbound ligands. Liver samples were collected and analyzed by confocal microscopy. WT mice were given IV PBS (panel C) or various doses of GFP-AdV5 (D-F) utilizing the same approach as described above, followed by confocal microscopy analyses or fluorimentry measurements of liver extracts (H) (503/535 nm).

FIG. 12 presents data concerning phase-contrast images of mouse cultured hepatocytes and LSEC at 24 hours after plating.

FIG. 13 presents data concerning of Alexa 633-Formaldehyde-treated Albumin FTALB) in WT mouse liver (2 hours after IV/RO inoculation). WT animals were R/O injected with Alexa633-FTALB. Two hours later, mice were euthanized, the livers were perfused to wash out unbound ligands for image analyses utilizing confocal microscopy.

FIG. 14 depicts the results of a Western blot assay of CAR protein expression in WT and hCD36 expressing HeLa cells. Panel A: Lane 1—WT HeLa, lanes 2 and 3-2 clones of HeLa-CD36 cells (CD36-1 and CD36-4). Panel B: Gene expression analyses of various AdV5 receptors by qRT-PCR. Gene expression levels were normalized by GAPDH and are presented as the ΔΔCt values vs WT cells (measurements were performed in duplicates).

FIG. 15 presents data concerning AdV5 binding to CD36 overexpressing and mock-transfected Hela cells. AdV5-GFP binding analysis was performed by incubating various concentrations AdV5 expressing vector with CD36 overexpressing HeLa cell, Clone 1 ( [symbol] ), Clone 4 ( [symbol] ) or mock-transfected HeLa cells (▪) for one hour on ice. The cells were washed with ice-cold PBS and further incubated in virus free DMEM media contacting 10 FCS for 24 hours at 37° C. in CO₂ incubator. GFP signal was measured utilizing Victor 3 fluorimeter.

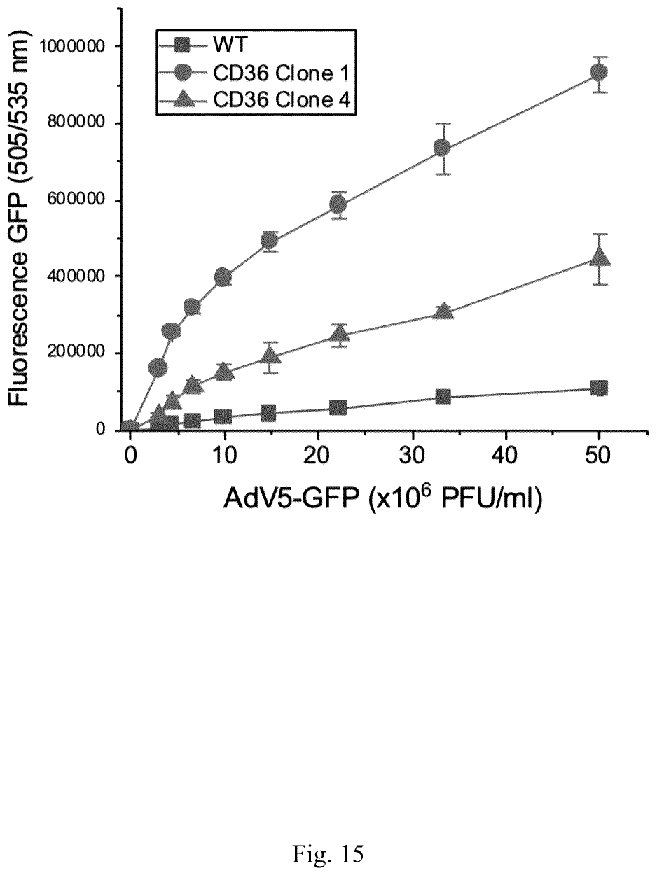

FIG. 16 presents data concerning cross-linking of AdV5 with CD36 in WT and CD36 overexpressing Hela cells. AdV5-GFP cross-linking analysis was performed by incubating 10¹⁰ PFU/ml of AdV5 expressing vector with CD36 overexpressing HeLa cell, Clone 1 (▪) or mock-transfected HeLa ( [symbol] ) with formaldehyde as a cross-linker. In parallel experiment cell extracts were prepared without formaldehyde crosslinking from CD36 overexpressing HeLa cell, Clone 1 ( [symbol] ) or mock-transfected HeLa ( [symbol] ). AdV5 associated CD36 was detected in ELISA method.

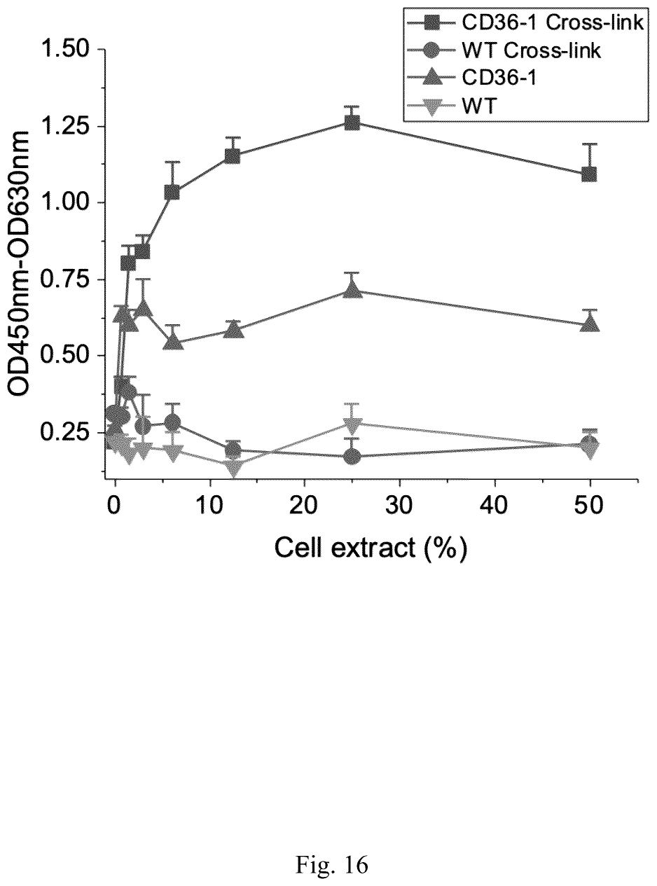

FIG. 17 presents data concerning the uptake of Alexa 488/568-AdV5 in NPLC. NPLC and hepatocytes were isolated from WT mice and cultivated for 24 hours as described in Methods. NPLC (Panels A, B) were incubated with Alexa488-AdV5, followed by a pulse-chase incubation with Alexa 568-transferrin (Panels B) or Lysotracker Red (Panels A), both red. Two lower panel are magnifications of panels A and B, respectfully.

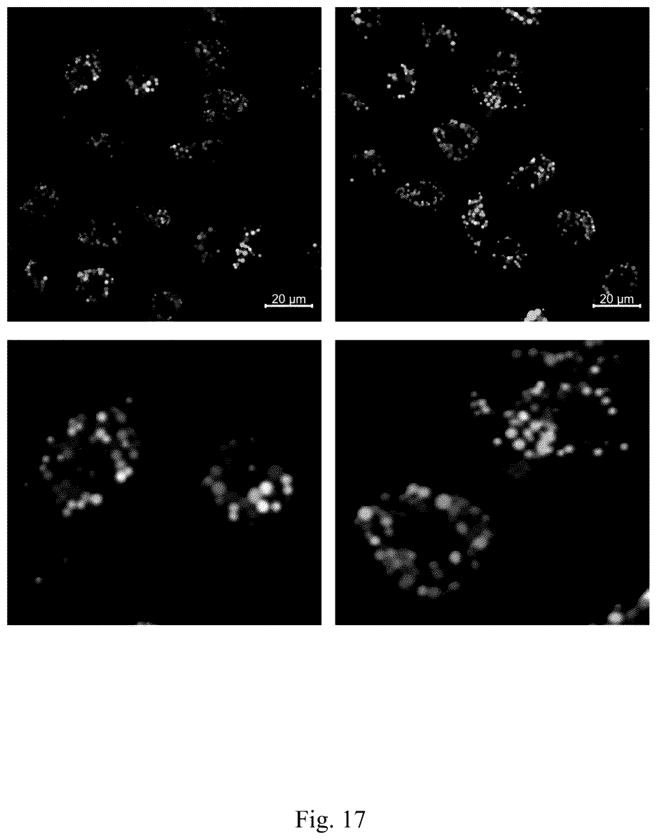

FIG. 18 presents data demonstrating that SR-Bs mediate Alexa 488 AdV5 uptake and its internalization to early endosomes. Cultured HeLa cells were incubated with 20 μg/ml Alexa 488-AdV5 in DMEM, 2 mg/ml fatty acid free BSA for 2 hours; media was changed for fresh containing 5 μg/ml of Alexa 568-transferrin (red) for another 30 minutes, washed with PBS and visualized utilizing CMS ex tempore.

FIG. 19 presents data concerning the Lipoprotein and anti-loop AB uptakes by human SR-BI and SR-BII. HeLa cells cultured on 12-well plates were incubated with none (A), 10 μg/ml of Alexa 488-labeled HDL (B) or LDL (C), or with 1×10⁷ pfu/ml of Alexa 488-labeled AdV5 (D) for 2 hours at 37° C. Cells were washed with PBS three times and detached utilizing a cell stripper solution. Cell-associated fluorescence was measured by FACS Analyzer.

FIG. 20 presents data concerning the role of SR-BI/II in AdV5 infectivity by GFP reporter gene in HeLa cells. Cultured HeLa cells were incubated with no, 1×10⁸, 1×10⁷, 1×10⁶ pfu of GFP-expressing AdV5 for 24 hours, cells were washed with PBS, detached with cell stripper and cell-associated fluorescence was measured by FACS.

FIG. 21 presents data demonstrating that recombinant adeno V5 mediates cytotoxicity in CD36/SR-BI and SR-BII mediated pathway. Various HeLa cells overexpressing hSR-B were plated at 75% of confluence, 24 hours later cells were added with 10⁸ PFU/ml of AdV5 or 10³/ml of Staph. Aureus, and further incubated for next 72 hours. HeLa cell layer resistance was measured.

FIG. 22 presents data concerning hepatocyte GFP expression mediated by GFP expressing recombinant AdV5 and Alexa 633 HDL uptake in various mice. WT, SR-BI/IIKO and CD36 KO were IV inoculated with 4×10⁶ PFU/g of GFP-AdV5. 22 hours later mice were additionally IV inoculated with 15 μg/ml of Alexa 633-HDL, and two hours later mice were sacrificed, perfused though inferior vena cava to wash out unbound ligands. Livers were removed to image utilizing in situ confocal microscopy.

FIG. 23 presents data concerning hepatocyte GFP expression mediated by GFP expressing AdV5 and Alexa 633 HDL uptake in various mice. WT, hSR-BI and hSR-BII transgenic mice were IV inoculated with 4×10⁶ PFU/g of GFP-AdV5. 22 hours later mice were additionally IV inoculated with 15 μg/ml of Alexa 633-HDL and two hours later mice were sacrificed, perfused though inferior vena cava to wash out unbound ligands. Livers were removed to image utilizing in situ confocal microscopy.

FIG. 24 presents data demonstrating that hSR-BI expression in SRBI/II mice restores AdV5-GFP clearance. SR-BUII KO mice were IV inoculated with 4×10⁶ PFU/g of empty AdV5 (Panel A) or hSR-BI-AdV5 (Panel B). At 72 hours after IV, mice received AdV5-GFP and then 22 hours later mice were additionally IV inoculated with 15 μg/ml of Alexa 633-HDL. Two hours later mice were sacrificed, perfused though the inferior vena cava to wash out unbound ligands. Livers were removed and imaged utilizing in situ confocal microscopy.

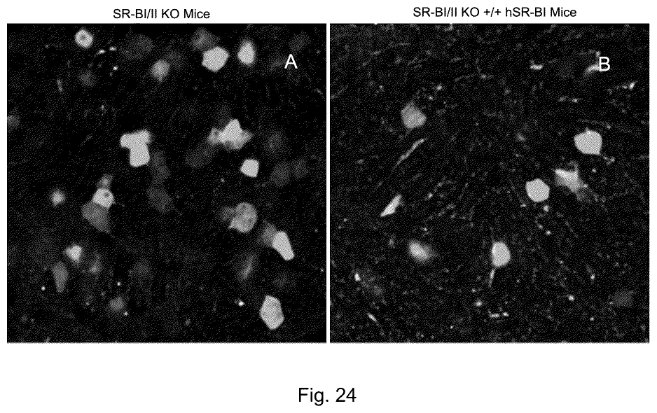

FIG. 25 presents data concerning the AAV2 and AAV8 uptake/infectivity in mock-transfected, SR-BI and CD36 overexpressing HeLa cells. Cultured HeLa cells were incubated with various concentrations of GFP-AAV2 and GFP AAV8 for two hours with 1×10⁷ pfu/ml of either of AVV at 37° C. Cells were then washed with PBS for three times and further incubated in DMEM containing 10% FCS for another 48 hours. In separate experiment cells were incubated with various concentrations of GFP-AAV2 and GFP AAV8 for 48 hours with 1×10⁷ pfu/ml of either of AVV at 37° C.

FIG. 26 presents data concerning ppLV-SARS-CoV2, ppLV-GVSV, ppLV-BALD infectivity in mock-transfected, SR-BI and CD36 overexpressing HeLa Cells. Cultured HeLa cells were incubated with various concentrations of ppLV-SARS-CoV2, ppLV-GVSV and ppLV-BALD for two hours with 1×10⁷ pfu/ml of either of ppLV at 37° C. Cells were then washed with PBS three times and further incubated in DMEM containing 10% FCS for another 48 hours.

FIG. 27 presents data concerning ppLV-SARS-CoV2 GFP transduction in SR-B deficient mice. WT (panel A), CD36 KO (panel B) or SR-BI/II KO (panel C) mice were IV inoculated with 4×10⁶ PFU/g of ppLV-SARS-CoV2 GFP, 22 hours later mice were additionally IV inoculated with 15 μg/ml of Alexa 568-HDL and two hours later mice were sacrificed, perfused though inferior vena cava to wash out unbound ligands. Livers were removed to image utilizing in situ confocal microscopy.

FIG. 28 presents data concerning MHV infectivity in mouse hepatoma BNL CL.2 and LR7 mouse fibroblast cell line transfected with AdV-Luc and AdV-hSRBI (CLA1) vectors. Mouse hepatoma cells (A) or mouse fibroblast cells (B) were transfected with AdV-Luc (control) and AdV-hSR-BI (CLA1) vectors for 48 hours before MHV treatment. Following incubation with various concentrations of MHV for two hours at 37° C., cells were washed with PBS twice and further incubated in DMEM containing 10% FCS for another 18 hours. Cells were washed with PBS, and cell-associated GFP fluorescence was measured using a Victor 3 multilabel plate reader.

FIG. 29 presents data concerning MHV uptake and infectivity in vector- and hSR-BI-transfected mouse hepatoma BNL CL.2. Mouse hepatoma cells were transiently transfected with either control or hSR-BI vectors before MHV treatment. For MHV uptake (A) cells were incubated with various concentrations of MHV for two hours at 37° C., washed with ice-cold PBS and further used for total RNA extraction and qRT-PCR assay of M-protein (MHV structural protein) gene expression. For assessment of MHV infectivity, cells were incubated with various concentrations of MHV for two hours at 37° C., then washed with PBS twice and further incubated in DMEM containing 10% FCS for another 18 hours. Cells were washed with PBS, and cell-associated GFP fluorescence was measured using a Victor 3 multilabel plate reader.

FIG. 30 presents data concerning MHV infectivity in primary culture of hepatocytes and liver sinusoidal epithelial cells (LSEC) isolated from WT and hSR-BI (CLA1) transgenic mice. Primary cultures of hepatocytes (A) and LSEC (B) from WT and hSR-BI transgenic mice were incubated with various concentrations of MHV in WE growth media (Williams' Medium E supplemented with insulin, dexamethasone and 10% FCS) for two hours at 37° C., then cells were washed with PBS two times and further incubated in WE growth media for another 18 hours. Cells were washed with PBS, and cell-associated GFP fluorescence was measured using a Victor 3 multilabel plate reader.

FIG. 31 presents data concerning MHV infectivity in bone marrow-derived macrophages from WT and SR-BI/BII-KO mice. Macrophages differentiated from bone marrow cells isolated from WT and SR-BI/BII-KO mice and cultivated for 5-7 days in RPMI growth media (RPMI supplemented with 10 ng/ml MCSF and 10% FCS), were incubated with various concentrations of MHV for two hours at 37° C. Cells were then washed with PBS and incubated in RPMI growth media for another 18 hours. Following washing with PBS cells we used either for measurement of cell-associated GFP fluorescence (A) or total RNA extraction and qRT-PCR assay of GFP (B) and M-protein (C) gene expression.

FIG. 32 presents data concerning the effects of apoA1-mimetic SAHPs and HDL on MHV infectivity in mouse hepatoma BNL CL.2. Mouse hepatoma cells were preincubated for 1 hour with various concentrations of L37 pA, ELKB and L-3D peptides or HDL, and with the added MHV (2.5×10⁶ PFU/ml) for the next two hours, in a serum-free DMEM media at 37° C. Following FCS addition to a final concentration 10%, cells were incubated for another 18 hours, then were washed with PBS, and cell-associated GFP fluorescence was measured utilizing a Victor 3 multilabel plate reader.

FIG. 33 presents data concerning the effect of SR-BI blocking antibody on MHV infectivity in mouse hepatoma BNL CL.2. Mouse hepatoma cells were first pre-incubated for 45 minutes with various concentrations of either anti-SR-BI blocking antibody or control non-immune antibody, followed by incubation with the added MHV (2.5×10⁶ PFU/ml) for the next two hours, in a serum-free DMEM media at 37° C. After FCS addition to a final concentration 10%, cells were incubated for another 18 hours. Cells were washed with PBS, and cell-associated GFP fluorescence was measured using a Victor 3 multilabel plate reader.

FIG. 34 presents data concerning the effect of class B scavenger receptor overexpression and knockdown in CALU3 cells on SARS-CoV-2 infectivity. Calu3 cells were transfected with siRNAs or gene encoding plasmids. After 48 h incubation, cells were infected with SARS-CoV-2 delta at MOI=0.1. After 24 h post infection, media were collected, and virus amount in the media were titered by plaque assay. The viral infectivity results under different conditions are graphically presented. Graph bar values are means+SD of 3 independent samples.

FIG. 35 presents data concerning the effect of amphipathic helical peptides on SARS-CoV-2 infectivity. Calu3 cells were prepared treated with virus as in the Materials and Methods section. 30 minutes prior to treatment with SARS-CoV-2 delta, L-37 pA, ELKB and L3D-37 pA were added at various concentrations and maintained during the entire incubation. Two aliquots of each well medium at 110 ul/aliquot were collected and virus in the media was tittered by plaque assay.

FIG. 36 presents data demonstrating that SARS-CoV-2-induced mortality in wild-type and SR-BI-KO, SR-BI heterozygous, CD36 KO and control mice. SR-BI KO (background Taconic C57BL/6), heterozygotes and control (Taconic C57BL/6) mice as well as CD36 KO (background Jackson Lab C57BL/6) and control (Jackson Lab C57BL/6) were treated with mouse adapted SARS-CoV-2 (strain MA10 from bei Resources) with a dose of 1e+05 (stock titer: 2.15e6 PFU/ml, 4.31e6 TCID50/ml) instilled intranasally and followed over 20 days. Weights were determined every 2-4 days. Mortality was determined by either death, a weight loss greater than 25% or meeting euthanasia criteria. The SR-BI experiment has been done twice, the CD36 experiment has been done once.

FIG. 37 schematically represents the experiments in Example 5 concerning cross-linking of AdV5 vector to CD36. On the left side of the figure is the experimental schematic as described in Example 5. On the right, detected CD36-AdV5 complexes from CD36 overexpressing and WT HeLa, with or w/o crosslinking are shown.

FIG. 38 presents data concerning CD36-mediated uptake of various ligands including AdV5. HeLa cells cultured on 12-well plates were incubated with nothing added (A), 10 ug/ml of Alexa 488-labeled LDL (B), oxLDL (C), or 10⁷ pfu/ml of Cy2-labeled AdV5 (D) at 37° C. for 2 hours. Cells were washed with PBS three times and collected utilizing a cell stripper solution. Cell associated fluorescence was measured by FACS Analyzer. Mock-Hela (E) and CD36-HeLa (F) cells cultured on glass slides were incubated with 20 μg/ml Alexa 488-AdV5 (green) in DMEM, 2 mg/ml Fatty Acid Free BSA for 2 hours, fixed and then stained for CD36 utilizing anti-CD36 antibody (E and F), and visualized utilizing confocal microscopy.

FIG. 39 presents data concerning blocking of AdV5 transduction utilizing anti-CD36 antibody and CD36 binding domain derived peptides in A549 cells. A549 cells were incubated with various antibody dilutions (left panel) or with CD36 binding domain derived peptide (right panel) in the presence of indicated concertation of AdV5-GFP for 24 hours in 10%-DMEM followed by PBS washing and GFP quantification as described in Example 5.

FIG. 40 presents data concerning the role of lipoproteins in AdV5-GFP-induced transduction in CD36 overexpressing and WT HeLa cells. Cells were incubated with various concentrations (10⁵-10⁸ pfu/ml) of AdV5-GFP vector in the presence or absence of 10% FCS, 40 μg/ml either or LDL for 1 hour at 37 C in BSA/DMEM, washed with PBS and additionally incubated for 24 hours in 10% FCS/DMEM followed PBS wash and GFP count.

FIG. 41 is a schematic representation of in vitro AdV5-GFP activity/degradation assay described in Example 5. Briefly, CD36-HeLa and Mock-HeLa were grown basic media until full confluence afterwards, cells were wash with Ca, Mg free PBS two times and further incubated with DMEM containing 2 mg/ml BSA with or without AdV5-GFP at 10¹⁰ PFU/ml for 1 hour in CO₂ incubator. The cells were washed with ice-cold free PBS three times and immediately added with ice-cold water, incubated 10 minutes and centrifuged 1000 g×10 min, cell extract was added in serial dilution to 96-well plates with cultured WT HeLa cell in 10% FCS/DMEM for 24 hours. The GFP signal was measured using a Victor 3 Fluorimeter.

FIG. 42 presents data concerning AdV5-GFP vector activity/degradation in WT and CD36 overexpressing HeLa cells. Cells were incubated with various concentrations (10⁵-10⁸ pfu/ml) of AdV5-GFP vector for 1 hour in CO₂ incubator. The cells were washed with ice-cold PBS three times and with ice-cold water then immediately added, incubated 10 minutes and centrifuged 1000 g×10 min, cell extract was added in serial dilution to 96-well plates with cultured WT HeLa cell in 10% FCS/DMEM for 24 hours for development of GFP expression. GFP signal was measured with a Victor 3 Fluorimeter. The two panels represent two different AdV5-GFP vector preparations.

FIG. 43 presents data concerning AdV5 luciferase transduction in rat hepatocyte, LSEC and KC culture from WT and CD36 KO animals. Cells isolated and plated as described in Example 5 were cultivated for 24 hours. Hepatocyte (panel A), LSEC (panel B) and KC (panels C) were incubated with various concentrations (10⁵-10⁸ pfu/ml) of luciferase expressing AdV5 particles. The cells were washed with PBS and further incubated in virus free media for 48 hours. Luciferase activity was measured in cell lysates.

FIG. 44 presents data concerning AdV5 transduction/infectivity as estimated by GFP expression in co-culture of LSECs and hepatocytes. NPLC and hepatocytes were isolated from WT (A, B) and CD36 KO (C, D, E) mice and cultivated for 24 hours as described in Methods. Hepatocyte-LSEC co-cultures were incubated with 10⁸ pfu/ml of AdV5-GFP. Panels A, C represent bright field microscopy of various cells and the detection of GFP signal is shown in panels B, D, E.

FIG. 45 presents data concerning WT mice who received IP PBS, L37 pA (10 and 60 mg/kg) 1 hour before IV injection of AdV5-GFP. 24 hours later, mice were sacrificed, and the liver was isolated and homogenized. The homogenate fluorescence was measured utilizing a Victor3 fluorimeter. The left panel compares 10 mg/kg of L37 pA to PBS and the right panel compares L37 pA at both 10 mg/kg and 60 mg/kg to PBS.

### DETAILED DESCRIPTION OF THE INVENTION

Viral infections include both unenveloped and enveloped viruses, with examples such as AdV, HIV, influenza virus, RSV, HPV, HSV, SARS-CoV-1, SARS-CoV-2, Marburg and Ebola viruses. Newly emerging infections, representing viral species related to the common cold such as coronaviruses (up to 35% of cases), can be associated with the appearance of novel particularly dangerous species such as SARS-CoV and SARS-COV-2 as well as adenoviruses due to mutagenesis and persistence in animal hosts. Recent pediatric hepatitis cases, some involving death, have been associated with adenoviral infections (although the causative agent is not yet certain). B scavenger receptors, including SR-BI, SR-BII and CD36, have been found to provide a novel anti-viral defense mechanism protecting parenchymal cells of various tissues from direct viral interaction and infection by mediating viral clearance by cells of the reticular endothelial system (RES) expressing these receptors. This discovery opens up new approaches for treating various frequently severe and sometimes fatal viral infections. As proof of principal, one approach is using recombinant adenovirus-based vectors expressing SR-BI, SR-BII or CD36. This represents a therapeutic gene therapy directed at facilitating viral clearance during infection. Infections such as SARS-CoV2 do not yet have targeted therapies that are safe, effective and widely applicable as are for other diseases. In the lung where there is significant class B scavenger receptor expression, peptides recognizing class B scavenger receptors may be used to both decrease viral infectivity as well as decrease pulmonary inflammation as peptides such as L37 pA have been shown to significantly decrease pulmonary inflammation. Directed lung applications such as through use of an aerosol may be extremely effective because of this dual peptide function.

In one aspect, the invention provides an improved method involving viral vector-mediated gene transfer to an animal. The improvement involves antagonizing or down-regulating the expression of an SR-B in the cells and tissues responsible for viral clearance. In one embodiment of the invention, an agent can be used that antagonizes an SR-B or down-regulates the expression of an SR-B in sinusoidal liver endothelial cells (LSEC) and liver Kupffer cells (KC). For example, the inventive method can involve (a) administering a viral vector comprising a desired transgene to an animal and (b) administering to the animal an agent that antagonizes an SR-B or down-regulates the expression of an SR-B.

The animal to be administered the agent (i.e., which antagonizes an SR-B or down-regulates the expression of an SR-B) is one undergoing viral vector-mediated gene transfer, i.e., involving administering a viral vector comprising a desired transgene to an animal. In this respect, the agent can be administered to the animal before administering the viral vector, after administering the viral vector, or concomitantly therewith.

The viral vector can be any type of vector, such as are commonly employed in the context of viral vector-mediated gene transfer. For example, the vector can be derived from an adenovirus (AdV) (including any suitable strain, such as type 5, type 26, type 35 as well as ChAdV3 and ChAdV63, an adeno-associated virus (AAV type 2 and type 8), a murine hepatitis virus (MHV), and lentivirus-based vectors, or other known to those of ordinary skill in the art to be suitable sources for constructing viral vector-mediated gene transfer vectors. In certain embodiments, the viral vector-mediated gene transfer vector can be selected from vectors derived from AdV5, AdV26, AdV35, ChAdV3, ChAdV63), AAV 2/8, MHVppLV-SarsCoV2, ppLV-GVSV (glycoprotein vesicular stomatitis virus) or retrovirus-based vectors.

For use in the inventive method involving antagonizing an SR-B, a suitable agent is administered to the animal undergoing viral vector-mediated gene transfer. The agent can comprise, for example, a synthetic amphipathic helical peptide (SAHP), an antibody targeting the SR-B, or a small molecule inhibitor (antagonist) of the SR-B.

In one embodiment involving upregulating SR-B activity in target cells, a suitable agent can be administered as a direct injection into a tissue/body space, directly exposed to the cells ex vivo or systemically. The agent can comprise, for example, a synthetic amphipathic helical peptide (SAHP) or viral vector mediated SR-B overexpression. In yet another embodiment, the method may involve the use of active principals which can be derived from or include asymmetric SAHPs that combine a viral vector specific binding domain with a CD36/SR-BI specific binding domain, optionally connected via a spacer. Such exemplary reagents are described in International Patent Publication WO 2023/168350 A2; Bocharov et al., J. Biol. Chem. 279(34), 36072-36082 (2004); and Bocharov et al., J Immunol. 197 (2): 611-619 (2016), the contents of each of which are incorporated herein in their entireties, for example.

In certain embodiments, the activate agents for use in the method of this invention include class A amphipathic helical peptides, e.g. as described in U.S. Pat. No. 6,664,230, and PCT Publications WO 2002/015923 and WO 2004/034977. Peptides comprising a class A amphipathic helix (“Class A peptides”), can also be useful in the treatment of one or more of the other indications described herein.

Class A peptides are characterized by formation of an α-helix that produces a segregation of polar and nonpolar residues thereby forming a polar and a nonpolar face with the positively charged residues residing at the polar-nonpolar interface and the negatively charged residues residing at the center of the polar face (see, e.g., Anantharamaiah (1986) Meth. Enzymol, 128: 626-668). It is noted that the fourth exon of apo A-I, a natural ligand for SR-B, when folded into 3.667 residues/turn produces a class A amphipathic helical structure.

One class A peptide, designated 18A (see, e.g., Anantharamaiah (1986) Meth. Enzymol, 128: 626-668) was associated with various patents describing it as an apoA-I mimic potently diminishing various inflammatory processes associated with acute and chronic inflammatory states.

In connection with embodiments in which the agent comprises an SAHP consisting from 18A peptide, its modifications including a monomer, dimer and trimer connected through additional proline amino acids, examples of such can be, for example, as described in published sources, or analogues of these. See, for example, International Patent Publication WO 2023/168350 A2; Bocharov et al., J. Biol. Chem. 279(34), 36072-36082 (2004); and Bocharov et al., J Immunol. 197 (2): 611-619 (2016), the contents of each of which are incorporated herein in their entireties. In some embodiments, the SAHP comprises two amino acid sequences selected from the group consisting of

(18A, SEQ ID NO: 1)
DWLKAFYDKVAEKLKEAF,
(18ELK, SEQ ID NO: 2)
EKLKELLEKLLEKLKELL,
(18ELR, SEQ ID NO: 3)
ERLRELLERLLERLRELL,

and a variant or derivative thereof, the two sequences being coupled to each other via a proline or an alanine. Examples of SAHPs for use in the inventive method involving antagonizing an SR-B include peptides having sequences such as

(L37pA, SEQ ID NO: 4)
DWLKAFYDKVAEKLKEAF-P-DWLKAFYDKVAEKLKEAF,
(5A, SEQ ID NO: 5)
DWLKAFYDKVAEKLKEAF-P-DWAKAAYDKAAEKAKEAA,
(P5A SEQ ID NO: 6)
DHLKAFYDKVACKLKEAF-P-NWAKAAYDKAAEKAKEAA,
(P5A C12/H2 SEQ ID NO: 7)
DWLKAFYDKVAEKLKEAF-P-DHAKAAYDKAACKAKEAA,
(ELK SEQ ID NO: 8)
EKLKELLEKLLEKLKELL-P-EKLKELLEKLLEKLKELL,
(ELK-B. SEQ ID NO: 9;)
EKLLELLKKLLELLKKLL-P-EKLLELLKKLLELLKKLL,
(ELK-B2 SEQ ID NO: 10)
EKLKELLEKLLELLKKLL-P-EKLKELLEKLLELLKKLL,
(ELK-C SEQ ID NO: 11)
EELKEKLEELKEKLEEKL-P-EELKEKLEELKEKLEEKL,
(ELK-C1. SEQ ID NO: 12)
EELKAKLEELKAKLEEKL-P-EELKAKLEELKAKLEEKL,
(ELK-C3 SEQ ID NO: 13)
EKLKELLEKLKAKLEELL-P-EKLKELLEKLKAKLEELL,
(ELK-C4 SEQ ID NO: 14)
EKLKAKLEELKAKLEELL-P-EKLKAKLEELKAKLEELL,
(ELK-D SEQ ID NO: 15)
EKLKALLEKLLAKLKELL-P-EKLKALLEKLLAKLKELL,
(ELK-D2 SEQ ID NO: 16)
EKLKELLEKLLAKLKELL-P-EKLKELLEKLLAKLKELL,
(ELK-E, SEQ ID NO: 17)
EWLKELLEKLLEKLKELL-P-EWLKELLEKLLEKLKELL,
(ELK-F, SEQ ID NO: 18)
EKFKELLEKFLEKFKELL-P-EKFKELLEKFLEKFKELL,
(ELK-F2, SEQ ID NO: 19)
EKFKELLEKLLEKLKELL-P-EKFKELLEKLLEKLKELL,
(ELK-G, SEQ ID NO: 20)
EELKELLKELLKKLEKLL-P-EELKELLKELLKKLEKLL,
(ELK-H, SEQ ID NO: 21)
EELKKLLEELLKKLKELL-P-EELKKLLEELLKKLKELL,
(ELK-I, SEQ ID NO: 22)
EKLKELLEKLLEKLKELL-A-EKLKELLEKLLEKLKELL,
(ELK-J, SEQ ID NO: 23)
EKLKELLEKLLEKLKELL-AA-EKLKELLEKLLEKLKELL,
(ELK-K, SEQ ID NO: 24)
DWLKAFYDKVACKLKEAF-P-DWAKAAYNKAAEKAKEAA,
(ELK-L, SEQ ID NO: 25)
DHLKAFYDKVAEKLKEAF-P-DWAKAAYDKAAEKAKEAA,
(ELK-A2, SEQ ID NO: 26)
EKLKAKLEELKAKLEELL-P-EKAKAALEEAKAKAEELA,
(ELK-AS, SEQ ID NO: 27)
EKLKAKLEELKAKLEELL-P-EHAKAALEEAKCKAEELA,
(ELR, SEQ ID NO: 28)
ERLLELLRRLLELLRRLL-P-ERLLELLRRLLELLRRLL,
(ELR-P-18A, SEQ ID NO: 29)
ERLLELLRRLLELLRRLL-P-DWLKAFYDKVAEKLKEAF,
(ELK-P-18A, SEQ ID NO: 30)
EKLLELLKKLLELLKKLL-P-DWLKAFYDKVAEKLKEAF,
(ELKB-P-18A, SEQ ID NO: 31)
EKLLELLKKLLELLKKLL-P- DWLKAFYDKVAEKLKEAF,

or the sequences of Table 1 shown here:

TABLE 1

| SEQ | | |
|---|---|---|
| ID | | |
| NO | Sequence | Name |
| 32 | DWLKAFYDKVAEKLKEAF | 18A |
| 33 | DWLKAFYDKVAEKLKEAF-P- | L37pA |
| | DWLKAFYDKVAEKLKEAF | |
| 34 | EKLKELLEKLLEKLKELL | ELK mono |
| 35 | ERLLELLRRLLELLRRLL | ELR mono |
| 36 | EKLLELLKKLLELLKKLL-P- | ELK-B |
| | EKLLELLKKLLELLKKLL | |
| 37 | EKLKELLEKLLELLKKLL-P- | ELK-B2 |
| | EKLKELLEKLLELLKKLL | |
| 38 | EELKEKLEELKEKLEEKL-P- | ELK-C |
| | EELKEKLEELKEKLEEKL | |
| 39 | EELKAKLEELKAKLEEKL-P- | ELK-C1 |
| | EELKAKLEELKAKLEEKL | |
| 40 | EKLKELLEKLKAKLEELL-P- | ELK-C3 |
| | EKLKELLEKLKAKLEELL | |
| 41 | EKLKAKLEELKAKLEELL-P- | ELK-C4 |
| | EKLKAKLEELKAKLEELL | |
| 42 | EKLKALLEKLLAKLKELL-P- | ELK-D |
| | EKLKALLEKLLAKLKELL | |
| 43 | EKLKELLEKLLAKLKELL-P- | ELK-D2 |
| | EKLKELLEKLLAKLKELL | |
| 44 | EWLKELLEKLLEKLKELL-P- | ELK-E |
| | EWLKELLEKLLEKLKELL | |
| 45 | EKFKELLEKFLEKFKELL-P- | ELK-F |
| | EKFKELLEKFLEKFKELL | |
| 46 | EKFKELLEKLLEKLKELL-P- | ELK-F2 |
| | EKFKELLEKLLEKLKELL | |
| 47 | EELKELLKELLKKLEKLL-P- | ELK-G |
| | EELKELLKELLKKLEKLL | |
| 48 | EELKKLLEELLKKLKELL-P- | ELK-H |
| | EELKKLLEELLKKLKELL | |
| 49 | EKLKELLEKLLEKLKELL-A- | ELK-I |
| | EKLKELLEKLLEKLKELL | |
| 50 | EKLKELLEKLLEKLKELL-AA- | ELK-J |
| | EKLKELLEKLLEKLKELL | |
| 51 | DWLKAFYDKVACKLKEAF-P- | ELK-K |
| | DWAKAAYNKAAEKAKEAA | |
| 52 | EKLKELLEKLLEKLKELL-P- | ELK-P-ELK |
| | EKLKELLEKLLEKLKELL | |
| 53 | ERLRELLERLLERLREL-P- | ELR-P-ELR |
| | ERLRELLERLLERLRELL | |
| 54 | DHLKAFYDKVAEKLKEAF-P- | ELK-L |
| | DWAKAAYDKAAEKAKEAA | |
| 55 | EKLKAKLEELKAKLEELL-P- | ELK-A2 |
| | EKAKAALEEAKAKAEELA | |
| 56 | EKLKAKLEELKAKLEELL-P- | ELK-AS |
| | EHAKAALEEAKCKAEELA | |
| 57 | DWLKAFYDKVAEKLKEAF-P- | 5A |
| | DWAKAAYDKAAEKAKEAA | |
| 58 | DHLKAFYDKVACKLKEAF-P- | P5A |
| | NWAKAAYDKAAEKAKEAA | |
| 59 | DWLKAFYDKVAEKLKEAF-P- | P5A C12/H2 |
| | DHAKAAYDKAACKAKEAA | |
| 60 | EKLKELLEKLLEKLKELL-P- | ELK-P-ELK |
| | EKLKELLEKLLEKLKELL | |
| 61 | ERLLELLRRLLELLRRLL-P- | ELR-P-ELR |
| | ERLLELLRRLLELLRRLL | |

or a variant or derivative thereof (e.g., typically sharing at least 9500 sequence identity with the foregoing. For example, the SAHP can be a monomeric, dimeric and tetrameric symmetric synthetic amphipathic helical peptide such as L37 pA (targeting all SR-Bs s), or ELK-B and ELK-B32 (preferably targeting CD36) as well as the asymmetric peptide 18ELK-B-P-18A.

In connection with embodiments in which the agent comprises an antibody targeting the SR-B, the antibody can be or comprise commercially-available reagents, such as an anti-CD36 polyclonal or recombinant antibody available from ABCAM, INC. (Cambridge, MA). Other examples include anti-SR-BI blocking antibody from NOVUS (NB400-113, example Figure), various blocking antibody produced as a result of in vivo immunization utilizing SR-BI, SR-BII and CD36 AdenoV5 expressing vectors followed by humanization (recombinant AB) for clinical use.

BTL-1 is a small molecule which can be potentially used for SR-BI blocking (Nieland et al., 2002; published in PNAS on Nov. 26, 2002, in vol. 99, no. 24, 15422-15427; available at: Discovery of chemical inhibitors of the selective transfer of lipids mediated by the HDL receptor SR-BI (pnas.org)). Additionally, oxidized Phospholipids are known to be antagonists/agonists/ligands for CD36 (Nieland et al., 2002).

An alternative approach to administering SR-B antagonists (such as the SAHP, anti-SR-B antibodies, or small molecule SR-B antagonists discussed above) involves the temporary vector mediated knockdown of SR-B receptors, for example using viral (e.g., AdV5) shRNAs targeting SRBI/BII and CD36, to accomplish comparable results as are reported in the Examples herein regarding SR-B knockout (KO) animals. In accordance with this embodiment, the inventive method comprises administering a suitable agent for impairing (down-regulating) the expression of an SR-B to the animal undergoing viral vector-mediated gene transfer. The agent can comprise, for example, an interfering RNA molecule (shRNA) targeting the mRNA sequence encoding the SR-B, a small molecule interfering with the SR-B gene expression, or other similar approach.

For use in the inventive method, shRNA targeting relevant coding sequences of SR-Bs can be designed by a person of ordinary skill in the art. In this respect, the target sequences for SR-Bs are known Moreover, experiments described in the Example 1 herein employed a strategy involving knocking down of CD36 and SR-BI. These target sequences can serve as a guide for designing suitable shRNA for use in the context of the present invention. Some examples of target sequence products for an shRNA approach include the following which are commercially available (Dharmacon) for CD36, SR-BI and LIMP2:

| | |
|---|---|
| SMARTpool, 5 nmol | Target sequence |

M-010206-01-0005, siGENOME Human CD36 (948) siRNA

| | |
|---|---|
| siGENOME SMARTpool siRNA D-010206-03, | CGACACAUAUAAAGGUAAA |
| CD36 | (SEQ ID NO: 62) |
| siGENOME SMARTpool siRNA D-010206-04, | GGAGACCUGUGUACAUUUC |
| CD36 | (SEQ ID NO: 63) |
| siGENOME SMARTpool siRNA D-010206-05, | CAUAGGACAUACUUGGAUA |
| CD36 | (SEQ ID NO: 64) |
| siGENOME SMARTpool siRNA D-010206-06, | GUAUUUGAAUCCGACGUUA |
| CD36 | (SEQ ID NO: 65) |

M-010592-01-0005, siGENOME Human SCARB1 (949) siRNA • SMARTpool, 5 nmol

| | |
|---|---|
| siGENOME SMARTpool siRNA D-010592-01, | GGACAAGUUCGGAUUAUUU |
| SCARB1 | (SEQ ID NO: 66) |
| siGENOME SMARTpool siRNA D-010592-02, | GAACUGCUCUGUGAAACUG |
| SCARB1 | (SEQ ID NO: 67) |
| siGENOME SMARTpool siRNA D-010592-03, | UGACUGGCCUGCACCCUAA |
| SCARB1 | (SEQ ID NO: 68) |
| siGENOME SMARTpool siRNA D-010592-04, | GGACAAACUGGGAAGAUUG |
| SCARB1 | (SEQ ID NO: 69) |

M-012087-00-0005, siGENOME Human SCARB2 (950) siRNA-SMARTpool, 5 Nmol

siGENOME SMART pool siRNA D-012087-01, SCARB2

| | | |
|---|---|---|
| Target Sequence: | GAUGAAAUCUUGUCCCUUA | SEQ ID NO: 70 |

| | |
|---|---|
| Mol. Wt. | Ext. Coeff. |
| 13,358.0 (g/mol) | 380,478 (L/mol-cm) |

siGENOME SMARTpool SiRNA D-012087-02, SCARB2

| | | |
|---|---|---|
| Target Sequence: | UCACUUGACUGGUGGAUA | SEQ ID NO: 71 |

| | |
|---|---|
| Mol. Wt. | Ext. Coeff. |
| 13,373.0 (g/mol) | 375,847 (L/mol-cm) |

siGENOME SMARTpool siRNA D-012087-03, SCARB2

| | | |
|---|---|---|
| Target Sequence: | UCACACAGUUGACGAAUUG | SEQ ID NO: 72 |

| | |
|---|---|
| Mol. Wt. | Ext. Coeff. |
| 13,373.0 (g/mol) | 374,334 (L/mol-cm) |

siGENOME SMARTpool siRNA 0-012087-04, SCARB2

| | | |
|---|---|---|
| Target Saquence: | GCCAAUAGGUGAGACAAUG | SEQ ID NO: 73 |

| | |
|---|---|
| Mol. Wt. | Ext. Goeff. |
| 13,388.0 (g/mol) | 370,418 (L/mol-cm) |

In some embodiments of this invention, other sequences which can affect SR-B, can be used for efficient SR-B knockdown or for interfering with effective protein synthesis of SR-B. Also, attenuation of SR-B expression can be achieved using a small molecule inhibitor of SR-B expression, such as, for example Indolinyl-thizole based agents (see ACS Med Chem Lett 2015, 6, 4, 375-380) and LXR activation (see Atherosclerosis 2012, 222(2): 382-9).

In another aspect, the invention provides a method of promoting viral clearance from an animal/or patient. The data provided herein and discussed in the Example below reveal that SR-Bs (exemplified via experiments involving CD36) constitute an anti-viral defense mechanism, which protects parenchymal cells of various tissues from a direct interaction with AdV5 by mediating LSEC viral clearance and potentially clearance in other cells.

Accordingly, the inventive method of promoting clearance of a virus comprises upregulating expression of one or more SR-Bs within an animal. The animal is one that either has a current viral infection or is at risk of contracting one. The virus to be cleared can be any desired target virus, such as AdV, HIV, influenza virus, RSV, HPV, HSV, SARS-CoV-1, SARS-CoV-2, Marburg and Ebola viruses but not limited by these specific viruses.

The inventive method involving up-upregulating expression of one or more SR-B can be achieved by administering to the animal an active principal that up-regulates one or more SR-B. Exemplary active principals can be derived from or include asymmetric SAHPs that combine virus specific binding domain with a CD36/SR-BI specific binding domain, optionally connected via a spacer. Such reagents are described in Bocharov et al., (2004) and Bocharov et al., (2016), for example. Alternatively, such active principles could include agents that direct viruses toward the CD36-mediated viral clearance/neutralization pathways.

In the context of the invention, the “animal” can be any species of animal amenable to viral infection or viral vector-mediated gene transfer. Such animals can be such of veterinary importance (e.g., cattle, sheep, goats, fowl, and the like), zoological importance (e.g., amphibians, reptiles, birds, mammals, such as induvial members of threatened or endangered species), laboratory specimens (e.g., mice, rats, etc.), and primates, including humans. In this respect, the inventive methods can be employed in the context of veterinary or medical treatment involving human or animal patients.

In practicing the inventive methods, the agent that antagonizes an SR-B or down-regulates the expression of an SR-B, the active principal that down-regulates one or more SR-Bs, and/or any viral vector comprising a desired transgene, can be administered to the animal within a pharmaceutical preparation including such agent, active principal, and/or viral vector and a pharmaceutically active carrier.

The carrier used in the inventive composition can be any of those conventionally used and is limited only by physio-chemical considerations, such as solubility and lack of reactivity with the agent, active principal, and/or viral vector, and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically and physiologically inert to the agent, active principal, and/or viral vector and one which has no detrimental side effects or toxicity under the conditions of use.

The choice of carrier and manner of formulation of the inventive composition will be determined in part by the particular agent, active principal, and/or viral vector of the invention and other active agents or drugs used, as well as by the particular method used to administer the agent, active principal, and/or viral vector. A variety of suitable formulations of the pharmaceutical composition, thus, can be employed in carrying out the inventive methods described herein. The following formulations for parenteral, subcutaneous, intravenous, intramuscular, and intraperitoneal administration are exemplary and are in no way limiting. One skilled in the art will appreciate that these routes of administering the agent, active principal, and/or viral vector are known and that more than one route can be used to administer a particular compound.

Injectable formulations are among those formulations that are preferred in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Toissel, 4th ed., pages 622-630 (1986)). The formulation can be formulated for injection by any desired route, such as via intravenous, intraperitoneal, intratumoral, or peritumoral injection.

Formulations suitable for injection include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The agent, active principal, and/or viral vector can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethylene glycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, and synthetic oils. Specific examples of oils include peanut, soybean, sesame, cottonseed, corn, olive, petrolatum, and mineral. Suitable fatty acids for use in parenteral and other injectable formulations include oleic acid, stearic acid, and isostearic acid. Ethyl oleate and isopropyl myristate are examples of suitable fatty acid esters.

Suitable soaps for use in injectable formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

Preservatives and buffers may be used. In order to minimize or eliminate irritation at the site of injection, such compositions may contain one or more nonionic surfactants having a hydrophile-lipophile balance of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Injectable formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

For use in the inventive methods and uses, any suitable dose of the agent that antagonizes an SR-B or down-regulates the expression of an SR-B, the active principal that up-regulates one or more SR-Bs, and/or any viral vector comprising a desired transgene can be administered to the animal. The appropriate dose and dosage will vary depending upon such factors as the animal's species, age, weight, height, sex, general medical condition, previous medical history, etc., which dose and dosage can be determined by a clinician (veterinarian or human physician) or laboratory personnel, as appropriate. While ideal dosing can be achieved on a case-by-case basis, for peptides such as the SAHPs discussed herein, suitable doses can be at least 1 mg/kg, such as at least 5 mg/kg or at least 10 mg/kg and can have an upper range of 60 mg/kg, such as 50 mg/kg, or 40 mg/kg. Thus, doses comprising 20 mg/kg, 30 mg/kg, 40 mg/kg may be suitably administered to an animal, particularly a human, in the performance of the inventive methods.

The following example further illustrates the invention but, of course, should not be construed as in any way limiting its scope. In brief, the experiments discussed in the Example demonstrate that SR-Bs, and particularly CD36, are AdV5 LSEC receptors that mediate viral clearance and decreases AdV5 delivery to the liver and lung parenchymal cells for productive infection, thus representing an antiviral defense mechanism in vivo.

### Example 1

The results of the experiments discussed in this Example demonstrate that CD36 can mediate AdV5 binding, internalization, infectivity and toxicity in phagocytic and epithelial cells and may also play a critical broader-based role in liver sinusoidal endothelial cells (LSEC)-mediated antiviral defense via viral clearance, potentially applicable to viruses other than AdV5. Additionally, these results reveal that CD36 functions differently depending on cell type and tissue in terms of viral recognition, infectivity in epithelial cells, as well as a viral clearance and antiviral innate immune responses in reticular endothelial cells (RECs), including LSEC. The results of these experiments demonstrate that CD36 can increase AdV5 cellular uptake and increase transduction in some cells but also reduce AdV5 vector transduction under certain conditions such as in the absence of fecal calf serum or lipoprotein particles or decrease viral activity/increase degradation in some cells. This multiplicity of effects allows manipulation/treatment to affect a specific desired change such as increased/decreased transduction or increased/decreased degradation.

### Materials and Methods

### Reagents

All media, serum preparations, cell trackers, reactive fluorescent dyes and antibiotics were obtained from Thermo Fisher Scientific. The anti-CD36 monoclonal antibody FA16 was purchased from Abcam Inc. (Cambridge, MA). Human HDL and LDL were isolated as reported previously. Extensively oxidized LDL (oxLDL) was prepared by incubation with 5 μM CuSO₄ at 37 C for 24 has previously described. Insulin, dexamethasone, Percoll, Opti Prep, FBS were from Sigma-Aldrich.

### Cell Cultures

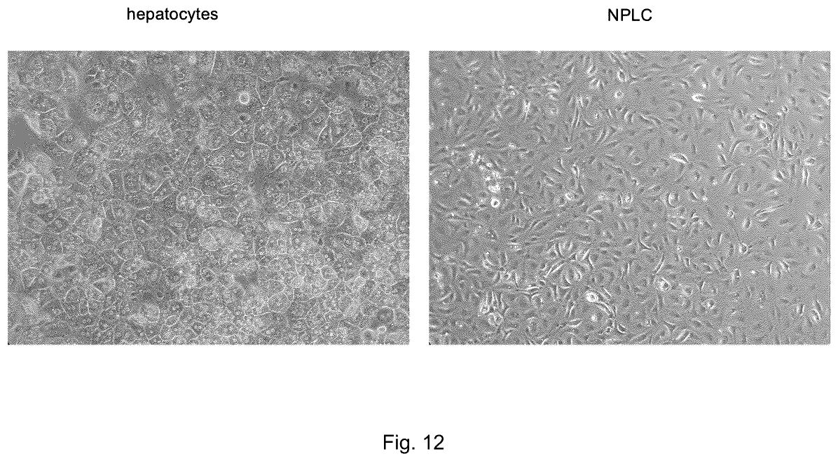

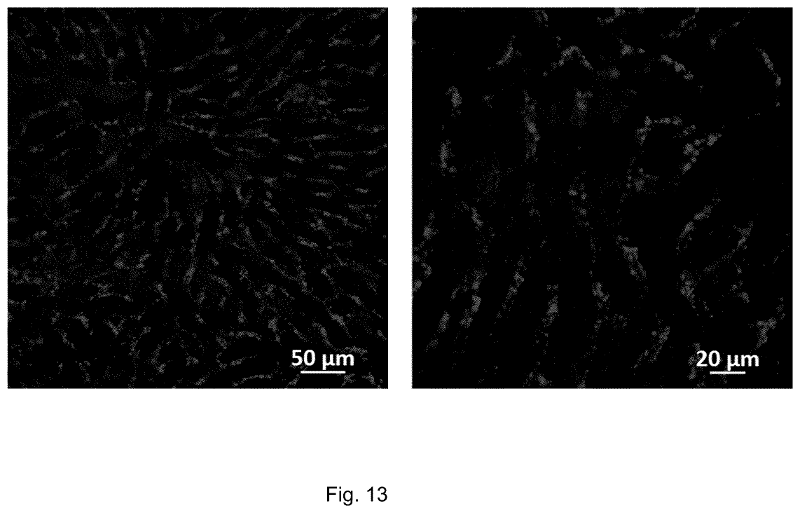

Stably transfected epithelial cell lines such as HeLa and HEK293 cells mock-transfected (Mock-HeLa and Mock-HEK) overexpressing human CD36 (CD36-HeLa and CD36-HEK) were characterized previously. Cells were grown in Dulbecco's modified Eagle's medium (DMEM) supplemented with 10% fetal calf serum (FCS), 2 mM glutamine, 100 IU/ml penicillin, 100 μg/ml streptomycin, and 100 μg/ml G418 at 37° C. in 5% CO₂ humidified atmosphere. NPLCs and hepatocytes were isolated from livers of CD36-deficient or wild type rats/mice using a method described previously with modifications where an Opti-Prep Density Gradient Reagent (Sigma) was used instead of Percoll for density gradient centrifugation for the isolation of NPLCs. Briefly, animals were anesthetized using ketamine/xylazine/acepromazine (80/10/0.02 mg/kg), the liver was perfused with 500 ml of Ca²⁺/Mg²⁺-free Hank's balanced salt solution (HBSS) followed by a perfusion with 15 ml of 1 mg/ml type I collagenase type 1 (Worthington, USA, cat #LS004197) in Mg²⁺-free HBSS containing 4 mM CaCl₂ at the rate of 3 ml/min. Liver cells were re-suspended in HBSS. Hepatocytes were sedimented by repetitive centrifugations at 50×g for 3 min in ice-cold HBSS. Supernatants containing sinusoidal non-parenchymal liver cells (NPLC) were collected and centrifuged at 300×g for 5 min. Pelleted NPLC were re-suspended in 17% of Opti-Prep Density Gradient Isosmotic Reagent solution in HBSS and centrifuged at 1500×g for 20 min. The NPLC, floated at the top of the gradient, were collected and pelleted by an additional centrifugation at 300×g for 5 min. The NPLC suspension was plated in serum-free DMEM into 96-well culture plates for 15 min to allow Kupffer cells (KC) to attach and spread. Non-attached cells were removed from culture plates by extensive washing, and the KC were further cultivated in RPMI 1640 containing 10% FCS and 10 ng/mL M-CSF. Supernatants containing LSEC as well as isolated HEP were plated on collagen coated plastic plates or glass slides in William's E media containing 10⁻⁶ M dexamethasone and 10 μg/ml Insulin (WEDI). After two hours the cells were washed with PBS and further cultivated in WEDI. Phase contrast images of hepatocyte (panel A) and LSEC (panel B) culture are seen in FIG. 12. Additionally, we characterize a LSEC compartment of mouse liver by an imaging for Alexa 633 labeled formaldehyde-treated BSA (LSEC-specific ligand) uptake (FIG. 13) in WT mice. As seen in this figure, Alexa 633 labeled formaldehyde-treated BSA demonstrates LSEC distribution without signs of its uptake by HEP.

### Fluorescent-Labeled Ligand Uptake and Reporter Gene Transduction Induced by AdV5 in Cell Culture

Adenovirus V5, native and oxidized lipoproteins and BSA were conjugated with Alexa 488/568, using a protein labeling kit (Invitrogen) following the vendor's instructions. HeLa cells, NPLCs and hepatocytes were incubated with Alexa488-AdV5 for 1 hour, followed by a pulse-chase incubation with Alexa 568-Transferrin or red Lysotracker, fixed with 3.7% paraformaldehyde and stained for anti-hCD36 FA16 antibody. Images (live and fixed) were analyzed using confocal microscopy. In some experiments HeLa cells were incubated with fluorescent-labeled ligands at 37° C. for 1 hour and then washed extensively with phosphate-buffered saline (PBS), detached with CELLSTRIPPER dissociation solution (Mediatech, Herndon, VA), fixed with 4% paraformaldehyde, and analyzed by a fluorescence-activated cell sorter (FACS, model A; Hitachi). Alternatively, cell associated fluorescence was measured utilizing a Victor 3 fluorimeter (PerkinElmer). For viral transduction assessment, preparations of adenovirus V5, expressing luciferase, GFP or mCherry reporters (1×10⁸-1×10⁶ PFU), were incubated for 24-48 hour with various cultured cells including HeLa, HEK293, HEP, LSEC and HEP-LSEC co-culture, and the viral infectivity/transduction was analyzed by Pierce™ Firefly Luciferase Flash Assay Kit (cat #16175, Thermo-fisher), fluorimentry (Victor 3 fluorimeter), fluorescent microscopy (Zeiss, OPTIMAX 7) or by confocal microscopy (Zeiss 780 confocal system). For AdV5 binding assessment, confluent cells were incubated with various concentrations of GFP-expressing AdV5 vectors (1×10⁸-1×10⁶ PFU) for 2 hours on ice. Afterwards, cells were washed with ice-cold PBS three times and further incubated in basic media for 48 hours. GFP expression was determined as written above.

### Adenovirus Cross-Linking in the Cell Culture

CD36-HeLa and Mock-HeLa were grown in basic media (DMEM, 10% FCS) to a full confluence. Cells were washed with Ca++, Mg++ free PBS two times and further incubated with DMEM containing 2 mg/ml BSA with or without AdV5-CFP at 10¹⁰ PFU/ml for 2 hours in CO₂ incubator. Afterwards, cells were washed with ice-cold Ca++, Mg++ free PBS three times and immediately added with pH 7.3 buffered PBS/10% formalin and incubated 10 minutes with slow rotation at room temperature (RT). The cells were washed with TBS to block cross linker and further lysed with extraction buffer (EB) 10 min at RT. Scrubbed cells and lysate were collected and further centrifuged 1000 g×10 min and used for ELISA based analysis. Briefly, 96-well ELISA plate were coated with anti-AdV5 mouse polyclonal antibody 5 μg/ml in 10 mM carbonate buffer pH 9.6 overnight at 4° C., washed with PBS, 0.1% Tween 20 (wash buffer-WB) 3 times for 3 minutes and block WB containing 2 mg/ml BSA for 1-2 hours at RT. After the next wash with WB once, plates were incubated with serial dilutions of cell extracts overnight at 4° C. Following additional washings with WB 3 times for 3 minutes at RT, plates were incubated for two hours with 2-5 μg/ml biotinylated anti-CD36 antibody in WB at RT, washed with WB 3 times for 3 minutes at RT and incubated with 2 μg/ml HRP-streptavidin for two hours in WB at RT, washed with WB 3 times for 3 minutes at RT. The reaction was developed utilizing ppCPP substrate.

Assessment of mRNA Expression for Several Known AdV5 Receptors in HeLa Cells, Overexpressing hCD36

RNA isolation and RT qPCR assays were performed as described above for mouse tissue samples. The following TaqMan Gene Expression assays (Thermo Fisher Scientific) were used: human CD36 (CD36, Hs00354519_m1), human CD46 (CD46, Hs00611257_m1), human integrin subunit alpha V (ITGAV, Hs00233808_m1), human integrin subunit beta 5 (ITGB5, Hs00174435_m1), human CAR (CXADR, Hs05395496_g1), human GAPDH (GAPDH, Hs02786624_g1).

Assessment of CAR Protein Expression in HeLa Cells Overexpressing hCD36 by Western Blot Analyses

For the Western Blot assay, cells lysates were prepared by direct adding 1× sample buffer (Life Technologies) to the wells with cultured HeLa cells (WT and CD36-overexpressing), with subsequent sonication and heating at 95° C. for 5 min. Samples were electrophoresed by reducing SDS PAGE, using 10% Tris-Glycine gels. Following protein transfer from gels to nitrocellulose membranes, blots were analyzed using the anti-human CD36 polyclonal antibody (Novus Biological, cat. #NB 400-145), and anti-CAR polyclonal antibody (Thermo Fisher Scientific, cat. #A302848A). Corresponding levels of 3-actin (control for sample loading) were assessed using 3-actin antibody (cat. #4967, Cell Signaling Technology).

### In Vivo Studies

For uptake experiments, WT and CD36-KO mice were injected retro-orbitally (R/O) or intravenously (IV) with 30 μg/g of Alexa488-HDL or with 4×10⁶ PFU/g of Alexa 488-AdV5. Two hours later, mice were euthanized, and the mouse liver, lungs and kidneys were perfused to wash out free ligands for confocal microscopy analyses. We imaged fluorescently labeled cells in intact tissues without sectioning using a Zeiss 780 confocal system (Zeiss, Jena, Germany). The tissues were kept cold and processed as soon as possible after collection. A piece of freshly excised liver tissue was washed in PBS and placed in 35 mm culture dishes with number 0 cover glass bottom (MatTek Corporation, Ashland, MA) for microscopy imaging analyses. Images were acquired sequentially by using a 488-nm laser line and emission wavelength between 505 and 580 nm for Alexa Fluor 488, a 693-nm laser line and emission wavelength between 638 and 747 nm for Alexa Fluor 647 and a 597-nm laser line and emission wavelength between 562 and 633 nm for Alexa Fluor 568. Tissue samples were also homogenized and extracted with 1% Triton X-100 in 50 mM Tris, pH 7.4. Fluorescence in tissue extracts, cleared by centrifugation, was also counted utilizing a Victor 3 fluorimeter.

AdV5 transduction/infectivity was analyzed by GFP, CFP and luciferase reporter expression after IV injection of 4×10⁶ PFU/g of GFP-AdV5 or LUC-AdV5 (Vector Biolabs). Twenty-two hours later, mice were further injected IV with 30 μg/g of Alexa 568 or Alexa 633-HDL (red), and two hours later, mice were anesthetized, sacrificed, perfused through the exterior vena cava to wash out free, unbound ligands. In separate experiments to test the effects of CD36 AdV5 vector driven GFP expression, mice were IV injected with 4×10⁷ PFU/g of corresponding vectors and 24 hours later, the GFP-AdV5 infective capacity was estimated by GFP reporter expression analyses as described above. All animal studies were approved by the Institutional Animal Care and Use Committee of the National Heart Lung and Blood Institute under protocols H-0050R2, H-0050R4 and H-0100R3.

### Assessment of GFP Expression Levels in Mouse Tissues by Fluorometry and Western Blot Analyses

Samples of excised tissues were quick-frozen on dry ice and stored at −80° C. The tissues samples were weighed, resuspended in 1:10 w/v of Tissue Protein Extraction Reagent I (Thermo Fisher Scientific) and disrupted using a chilled glass-Teflon homogenizer. The resulting lysates were centrifuged at 10,000×g for 10 minutes to pellet tissue debris. The supernatants were collected, and fluorescence intensity in the supernatants was measured using a Victor 3 multilabel counter. For Western Blot assay, aliquots of liver supernatants were mixed with 2× sample buffer (Life Technologies), heated at 95° C. for 5 min and electrophoresed by reducing SDS PAGE, using 10% Tris-Glycine gels. Following protein transfer from gels to nitrocellulose membranes, GFP blots were analyzed using an anti eGFP monoclonal antibody (F56-6A1.2.3, Thermo Fisher Scientific) and Alkaline Phosphatase Chromogenic Substrate (Life Technologies, cat. #WP20001). We also assessed levels of CD36 and β-actin by Western Blot using mCD36 antibody (cat. #AF2519, R&D Systems) and j-actin antibody (cat. #4967, Cell Signaling Technology).

Total RNA Isolation and Quantitative Real-Time PCR Analysis of eGFP mRNA in the Tissues of AdV5-GFP Treated Mice

For RNA isolation, tissue samples preserved in RNA-later solution were homogenized in Trizol Reagent using a Precellys 24 homogenizer (Bertin Technologies, Montigny-le-Bretonneux, France). RNAs were isolated using the PureLink RNA Mini Kit (Thermo Fisher Scientific). After the DNase treatment, RNA (2 g) was reverse transcribed using a TaqMan Reverse Transcriptase Reagent Kit. Real-time qPCR assays were performed on a StepOne Real-Time PCR System (Applied Biosystems), using 40 ng cDNA per reaction. TaqMan Gene Expression assays for eGFP were designed by Thermo Fisher Scientific TaqMan Assay custom design service. Relative levels of gene expression were measured by the comparative CT method with mouse GAPDH (TaqMan Assay ID number Mm03302249_m1, Thermo Fisher Scientific) as a reference gene. eGFP gene expression results were analyzed using the 2^{-ΔΔCT} formula and presented as normalized fold changes, compared to corresponding non-treated controls.

### Electron Microscopy

Confluent monolayers of HeLa cells were incubated with AdV5-LUC at 10⁸ PFU/ml for 2 hours in a CO₂ incubator. After three washings with ice-cold PBS, cells were fixed in PBS-buffered 2.5% glutaraldehyde (PBSG), scrubbed into the PBSG followed by double fixing in PBSG and osmium tetroxide (0.5%), dehydrated, and embedded into Spurr's epoxy resin. Ultrathin (90-nm) sections were double stained with uranyl acetate and lead citrate and viewed in a Philips CM10 transmission electron microscope (TEM) (Philips Electronics, Mahway, NJ).

### Cellular Cytotoxicity Assays

Cells were aliquoted into ACEA 96-well e-plates and allowed to sediment at room temperature for 30 min. The plates were then inserted into analyzing slots of the Real-Time Cell Analyzer (RTCA, ACEA Biosciences, San Diego, CA). Plates were cultured for 24-96 h until cells (various genetically modified HEK 293 cells) reached confluency and then, AdV5 was added at a concentration of approximately 1×10⁸ PFU/ml. Cell layer resistance was analyzed for the next 1-72 h. AdV5-induced cytotoxicity was measured using CytoTox 96® Non-Radioactive Cytotoxicity Assay (Promega, cat #G1780) according to the manufacturer's protocol. 10 ul of 10× lysis solution (per 100 ul of media) was added to the positive control wells to generate maximum LDH release. To calculate percent cytotoxicity for each experimental well, the following formula was used: Percent cytotoxicity=100× Experimental LDH Release (OD₄₉₀)/Maximum LDH Release (OD₄₉₀).

### Statistical Analyses

All data are expressed as mean values±standard deviation (SD). Differences between groups were analyzed by the Student's t-test, p<0.05 was considered significant.

### Results

### Uptake of Alexa 488-Labeled Adenovirus V5 is Increased in HeLa Cells Stably Expressing CD36

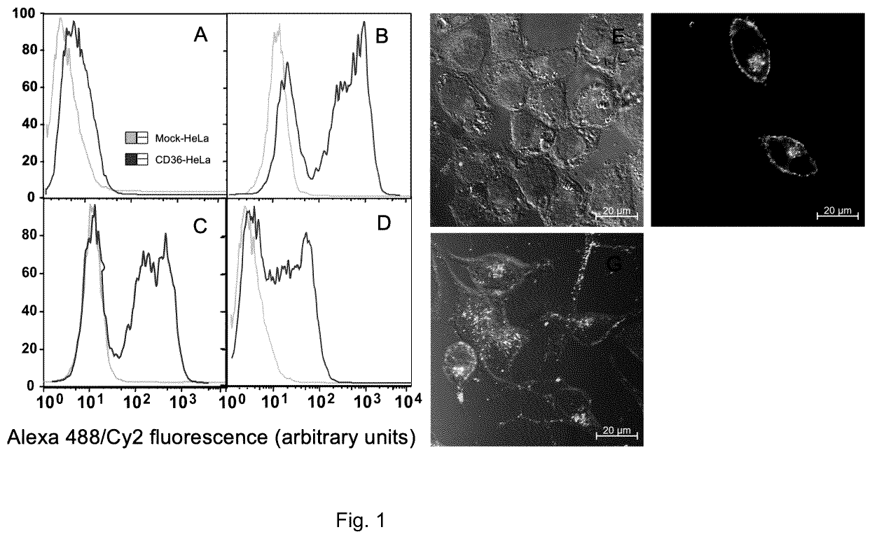

To investigate CD36 as a potential role as an adenoviral receptor, we first evaluated uptake of Alexa 488-protein labeled AdV5 in HeLa cells stably transfected with CD36 (CD36-HeLa). Using FACS analyses, we found that CD36 overexpression increased uptake not only of well-established CD36 ligands such as LDL and oxLDL, but also Cy2-AdV5 by 24-fold as judged by fluorescent peak shift at FACS graph of Cy2-AdV5 uptake in CD36-HeLa (red line) compared to a mock-HeLa control (green line) (FIG. 1 A-D). Furthermore, confocal microscopy analyses confirmed that surface binding and uptake of Alexa488-AdV5 (green) were increased in HeLa cells overexpressing hCD36 compared to mock-HeLa cells (FIG. 1 E, F). Alexa568-transferrin/alexa488-AdV5 colocalization in CD36 overexpressing HeLa cells was observed (FIG. 1G), indicating that CD36-mediated uptake was in part associated with AdV5 endocytosis into the endocytic recycling compartment (ERC). Colocalization of AdV5-Alexa488 with Lysotracker was also observed in CD36-HeLa cells, demonstrating a small but sizable AdV5 transport to lysosomes (LS).

This study was not designed to quantitively analyze a role of CD36 in AdV5 intracellular transport but, the data nonetheless demonstrate CD36-dependent AdV5 internalization into the ERC and LS in epithelial cells.

### AdV5 Particle/Virion Localization in CD36 Expressing HeLa Cells

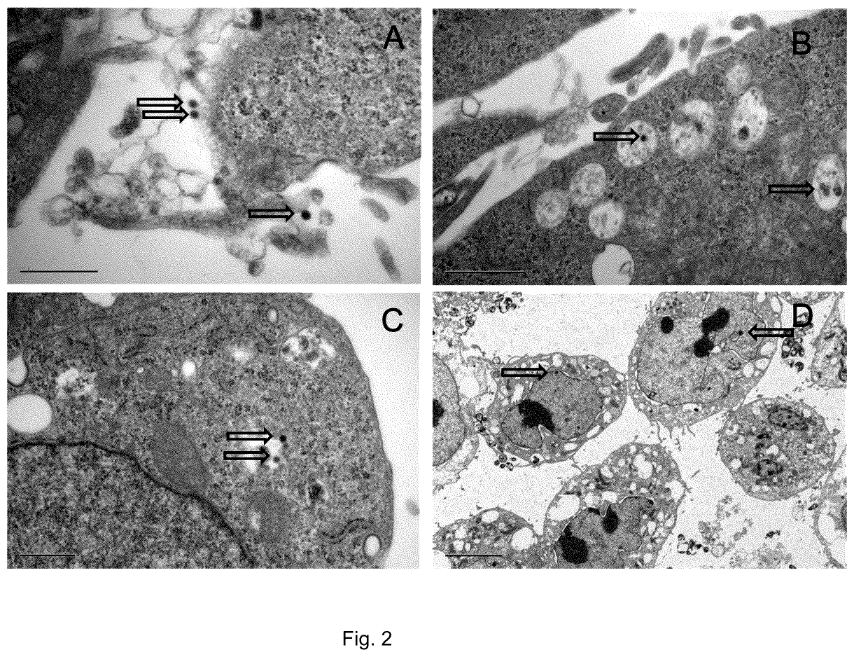

AdV5 uptake in CD36-HeLa cells was further analyzed using transmission electron microscopy (TEM). It was previously demonstrated that AdV5 particles are readily visible as 50-100 nm round black entities in cytosol, endocytic and mitochondria compartments, while no complementary antibody-dependent staining was required for their visualization. In FIG. 2, after initial adhesion to the cell surface, AdV5 was internalized in HeLa cells overexpressing CD36. The AdV5 particles are seen in endocytic vesicles (FIG. 2 B, arrow) cytosol (panel C), and some material, potentially, AdV5 DNA, in nucleus (Panel D), respectively, suggesting enhanced internalization and LS escape in CD36 expressing HeLa cells. This indicates that CD36 expression is an important regulator of AdV5 uptake in epithelial cells that are present in various tissues including the liver, lung, kidney, as well as pancreas and brain, which are common targets of gene therapy and AdV5 mediated vaccination.

### CD36 Increases Adenoviral Transduction in HeLa Cells

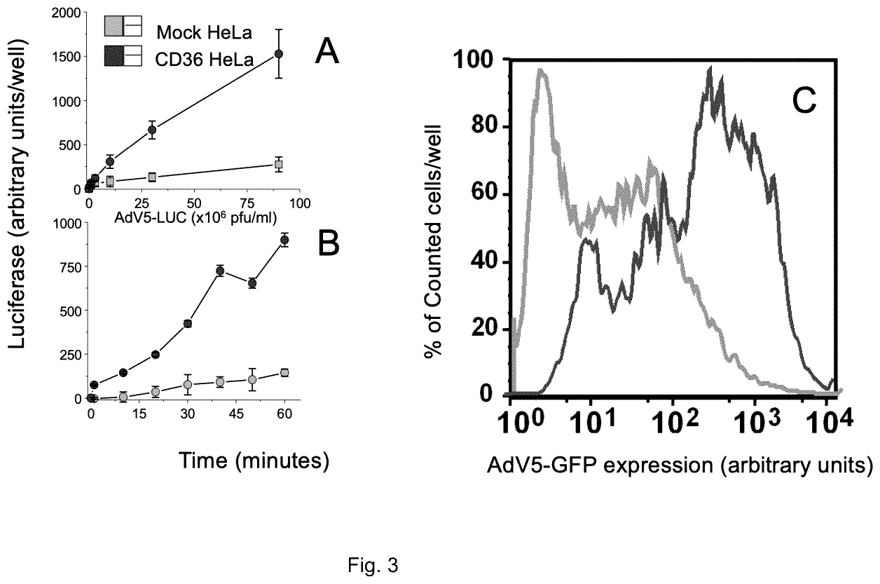

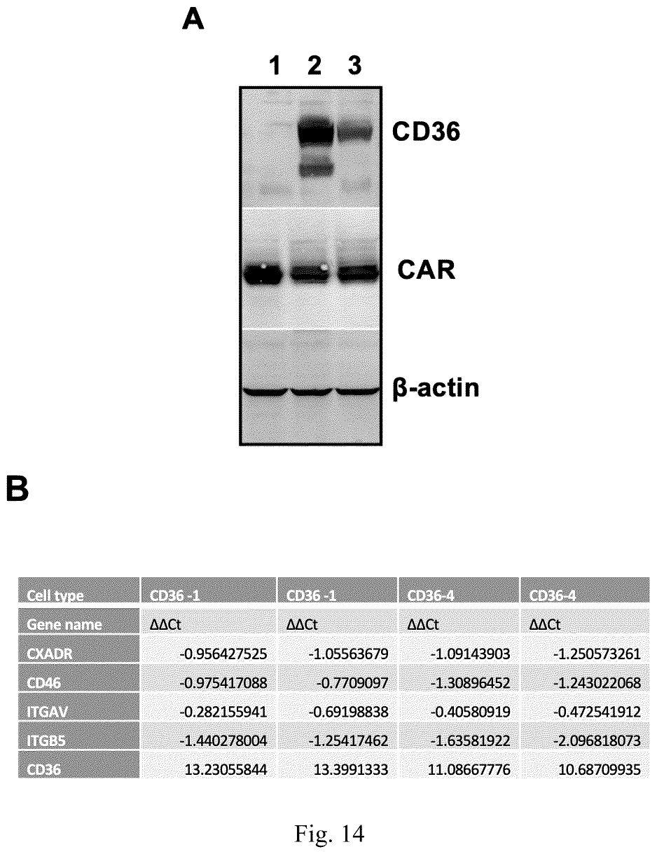

To assess the role of CD36 in adenoviral infectivity, CD36-HeLa were infected with GFP or luciferase reporter expressing AdV5 (AdV5-GFP or AdV5-LUC, respectively). As seen in FIG. 3 (A, B), CD36 overexpression resulted in a dose-dependent (FIG. 3 A) and time-dependent (FIG. 3B) increased expression of the luciferase reporter. Up to a 6-fold higher activity of AdV5-LUC infection at 1 h was observed in CD36-HeLa compared to mock-HeLa cells (FIG. 3B). In addition, AdV5-GFP transduction/infection levels measured by GFP expression were increased ˜10-fold in CD36-HeLa compared to mock-HeLa controls when analyzed by FACS (FIG. 3C). We had expanded this approach in accordance with previous reports to demonstrate CD36-dependent binding of AdV5 in CD36-HeLa while comparing it to Mock-HeLa cells (FIG. 14). After allowing AdV5 to bind to HeLa cells on ice for 1 hour, we conducted a PBS wash, followed by a 24-hour incubation period to enable GFP protein expression. CD36-expressing HeLa cells demonstrated a 5-fold increase in clone 1 and a 10-fold increase in clone 4, both of which expressed CD36. Of note, higher expressing clone 4 was more effective in AdV5-cold binding than CD36 clone 1 or Mock-HeLa cells (FIG. 14). This demonstrates that AV5 binding is associated with the CD36 receptor expression and function.

hCD36 Overexpression in HeLa Cells does not Affect the Expression of CAR Protein

To verify whether the hCD36 expression results in any changes in the levels of CAR protein, another well-known AdV5 receptor, we compared its expression in WT and hCD36 HeLa cells using Western blotting assay. Our data (FIG. 12) indicate that with the increased expression of hCD36 the expression of CAR protein remained unchanged. In addition, the results of qRT PCR analyses demonstrated that mRNA expression of CAR (cxadr gene) as well as several other known AdV5 receptors, in CD36 HeLa cells was either unchanged or moderately lower compared to WT HeLa cells (FIG. 13).

### CD36 Mediates Adenoviral Toxicity in HEK 293 Cells

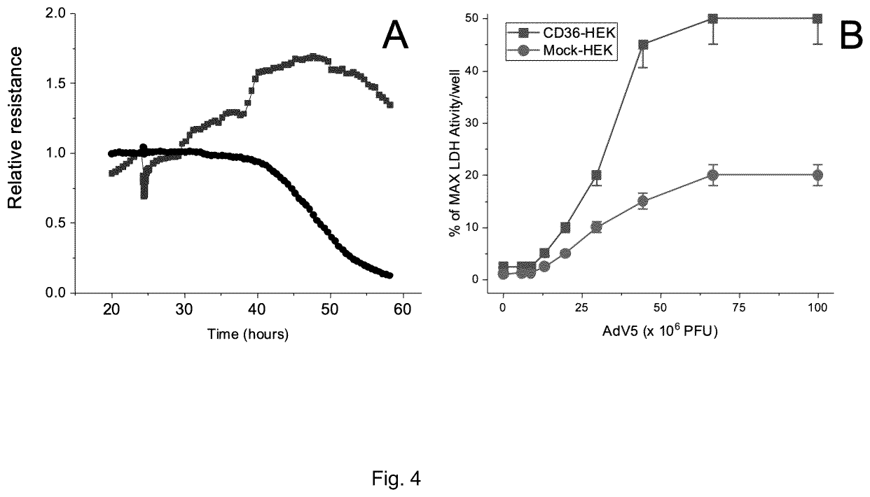

Because it is known that adenovirus-based vector administration is associated with toxicity, AdV5 toxicity was also analyzed in HEK293 cells stably transfected with CD36 (CD36-HEK/Mock-HEK). Increased CD36 expression was clearly associated with higher cytotoxicity in HEK293 cells. As shown in FIG. 4A, CD36 expression demonstrated a 50% reduction of monolayer integrity (corresponding to cellular toxicity) at 48 hours while no detectable cytotoxicity was found in mock-HEK293 cells treated with 1×10⁸ PFU of LUC-AdV5, when measured by RTCA. In addition, AdV5 treatment of CD36 overexpressing HEK-293 cells demonstrated a dose-dependent LDH release with up to 2.5-fold increase compared to mock-HEK (FIG. 4B).

CD36 Deficiency is Associated with a Reduced LSEC AdV5 Uptake in CD36 KO Mice

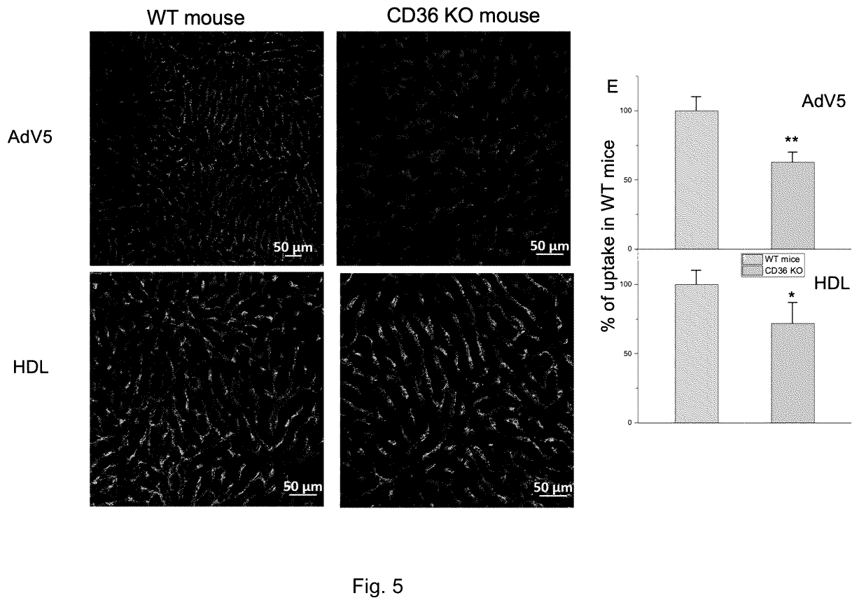

Since the gain of function in vitro experiments suggested that CD36 was an AdV5 receptor, mediating AdV5 binding and reporter transduction, we further analyzed AdV5 uptake and infectivity in vivo. As shown in FIG. 5, both Alexa488-HDL and Alexa488-AdV5 were taken up by liver sinusoidal endothelial cells, with minimal accumulation in hepatocytes observed at the 2-hour mark following intravenous (IV) injection. The distribution pattern of both labeled HDL and AdV5 closely resembled that of well-known markers of liver sinusoidal endothelial cells, including formaldehyde-treated albumin and ox-LDL uptake, as reported previously. This distribution pattern of HDL and AdV5 in liver sinusoids corroborates earlier findings regarding HDL and blood-borne AdV5 and HIV vectors. More importantly, HDL and AdV5 uptake by LSEC uptake in CD36 KO mice (FIG. 5 B, D), were reduced by ˜25% and ˜40%, respectively (FIG. 5E), when compared to WT controls (FIG. 5 A, C).

### AdV5 Uptake and GFP/LUC Gene Reporter Transduction in Cultured Hepatocytes, LSEC and Kupffer Cells

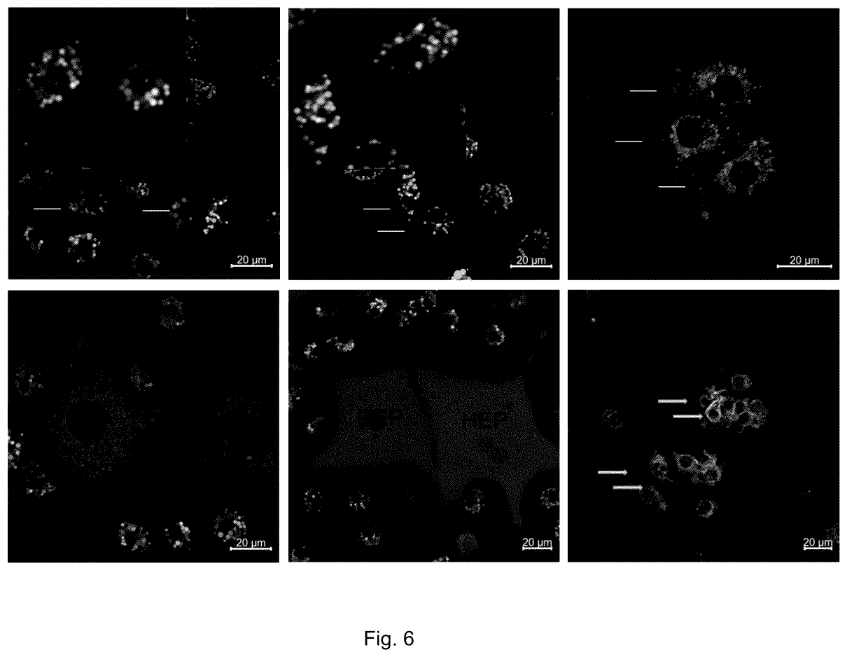

Since in vivo uptake of AdV5 occurred in sinusoidal liver cells, non-parenchymal liver cells (NPLC), which are composed of KC [10-20%] and LSEC [80-90%], rather than in hepatocytes (HEP), we further analyzed Alexa488-AdV5 uptake and GFP-AdV5/LUC-AdV5 transduction in cultured HEP, LSEC, Kupffer cells and NPLC. As shown in FIG. 6, mouse NPLCs were primarily responsible for uptake and endocytosis of AdV5 into the ERC (panels A, D). Partial sorting to lysosomes (panels B, E) was observed in both settings, NPLC primary culture (FIG. 6 A-C, F) and their co-culture with hepatocytes (FIG. 6 D, E). As shown in FIGS. 6 C and 6 D, the endocytosis of AdV5 was observed in NPLC with the expression of both CD36 (panel C) and the endothelial cell marker, CD31 (panel F).

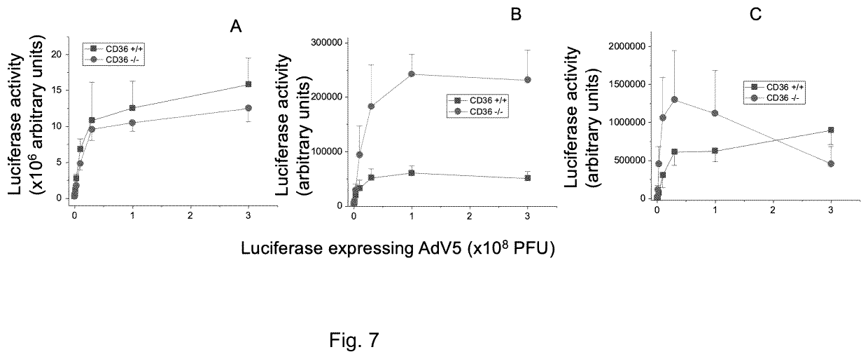

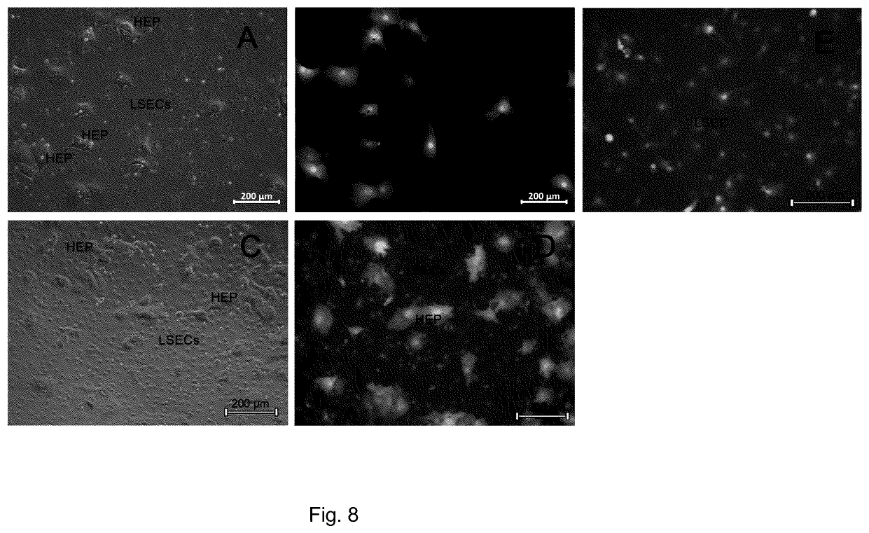

To further explore the viral transduction activity on these cells, cultured rat HEP, LSEC and Kupffer cells were transduced with LUC-AdV5. As shown in FIG. 7, no significant differences were observed of LUC-gene expression in HEP (FIG. 7 A) isolated from CD36 KO or WT rat. In contrast, LSEC (FIG. 7 B) and Kupffer cells (FIG. 7 C) isolated from CD36 KO rat were prone to LUC-AdV5 transduction compared to WT rat. LUC expression in LSEC and Kupffer cells were dose-dependent and demonstrated 6-fold and 2-fold increases in CD36 KO rat, respectively, when compared to WT rat. However, at higher AdV5 concentrations, Kupffer cells did not show differences in LUC expression. In co-culture of NPLC/LSEC isolated from both WT and CD36 KO mice with low hepatocyte content (FIG. 8 A-E), only hepatocytes expressed the GFP reporter upon transduction with GFP-AdV5. This indicates that hepatocytes were the primary target for AdV5 in WT mice. In contrast to WT mice, NPLCs showed resistance to AdV5 infection, despite being the primary cell type responsible for virus uptake. This suggests that NPLCs might neutralize the virus by transporting it to lysosomes, resulting in the lower GFP transduction observed (FIG. 8B). However, NPLC/LSEC cells from CD36 KO mice, when transduced with GFP-AdV5, expressed GFP (FIG. 8 D, E), which was more evident at higher magnifications (FIG. 8 E). At the same time, hepatocytes from CD36 KO mice (FIG. 8 D) expressed a similar level of GFP compared to WT hepatocytes (FIG. 8 B).

CD36 Protects the Parenchymal Cells of Liver and Lung from AdV5 Infection

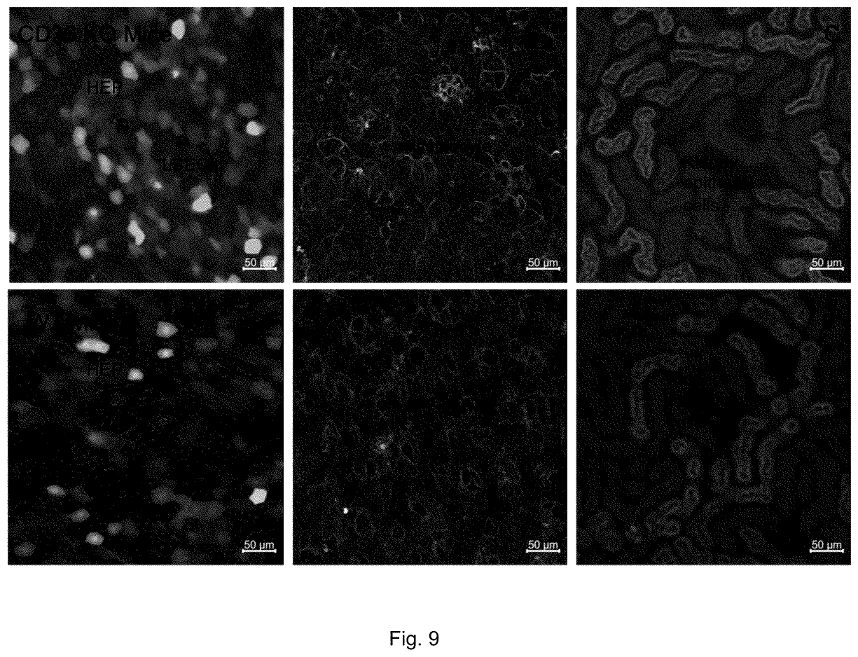

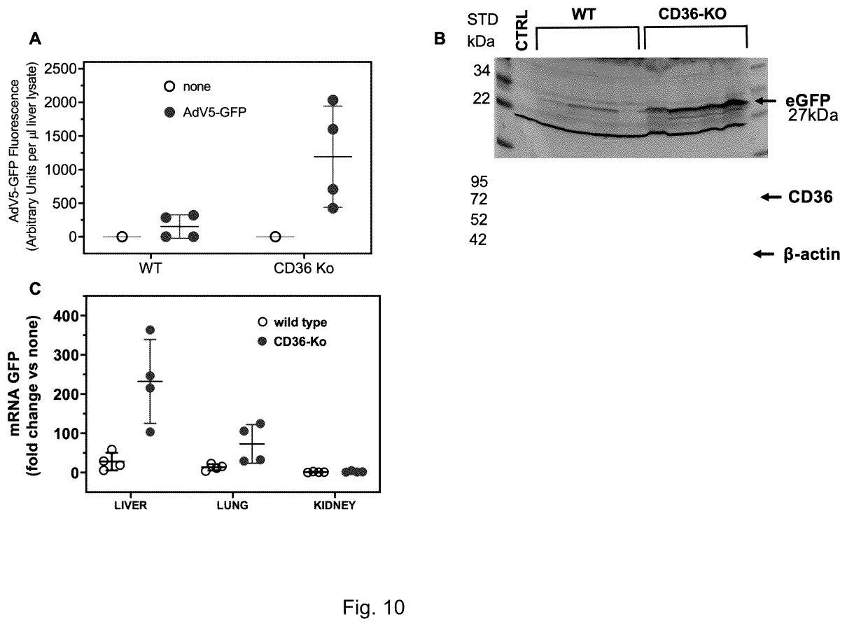

Since we found that NPLC from CD36 KO mice, especially LSEC, were prone to infection and AdV5 uptake was partially mediated through CD36, we analyzed AdV5-GFP transduction in organs of CD36 KO mice compared to WT mice after intravenous injection of 4×10⁶ PFU/g GFP-AdV5 in PBS. As seen in FIG. 9, parenchymal/epithelial cells (green signal corresponding to GFP expression) rather than non-parenchymal, endothelial cells (EC) expressed GFP. With EC of the liver and lung, Alexa 568-HDL was seen as a red signal that did not co-localize with the green signal. In kidneys, parenchymal tubular epithelial cells demonstrated both uptake of Alexa488-HDL and AdV-GFP transduction in CD36 KO mice with little difference in GFP signal compared to WT mice. However, importantly, the liver and lungs of CD36 KO mice appeared to be less protected as parenchymal cells of CD36 KO (FIG. 9 A, B) were highly transduced by GFP-AdV5 compared to WT mice (FIG. 9 D, E). Little differences were seen in kidneys where GFP expression was barely detectable (FIG. 9 C, F). The observation was further verified by lysate GFP fluorimentry (FIG. 10A), anti-eGFP western blotting (FIG. 10B), and quantitative RT-PCR of GFP gene expression in liver, lung and kidney (FIG. 10C). All three measured values (WB, GFP and RT-PCR) confirmed ≈10-fold increase of AdV5-GFP transduction in CD36 KO liver and lungs compared to WT mice.

### CD36 Restoration in CD36 KO or CD36 Overexpression in WT Mice Lead to a Protection Against GFP AdV5 Transduction

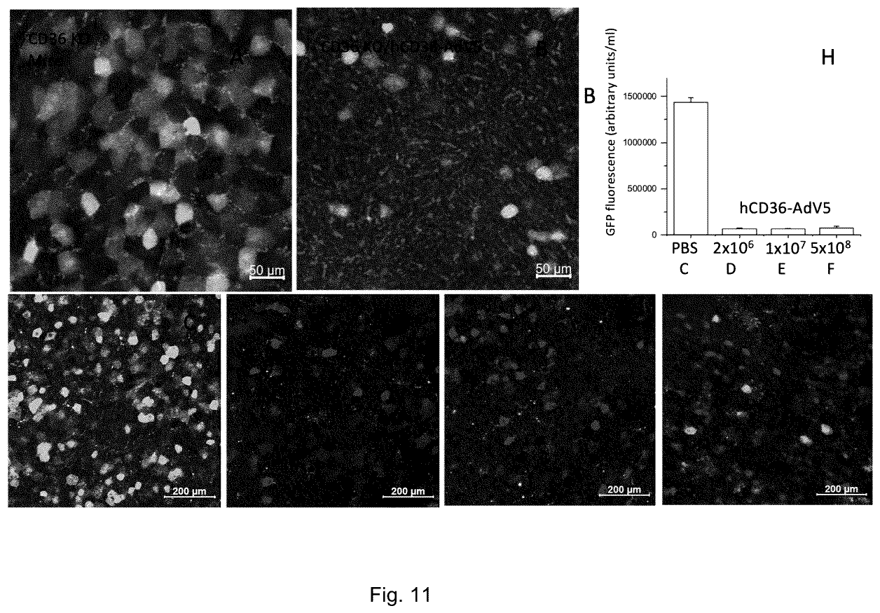

To further verify the role of CD36 in antiviral defense, CD36 KO mice were transfected with hCD36-AdV5 and LUC-AdV5 as a negative control. Two days later mice were infected with GFP-AdV5, and the hepatic GFP reporter expression was analyzed 24 hours later after an additional IV injection of Alexa 633/or Alexa 568-labeled HDL, utilizing confocal microscopy. The hepatocyte GFP expression was dramatically reduced following the hCD36-AdV5 transduction (FIG. 11B), compared to the control Luc-AdV5 (FIG. 11A) infection. Simultaneously, Alexa633-HDL uptake was increased in CD36 transduced mice (FIG. 11 B) compared to control (FIG. 11 A). These observations demonstrated that hCD36-AdV5 vector-mediated hCD36 expression resulted in a significant increase of Alexa 633-HDL uptake in the liver (panel B), and likewise, hCD36-AdV5 vector transduction greatly reduced subsequent GFP-AdV5 transduction in mice, suggesting an increased viral clearance by of NPLC/LSEC (FIG. 11 B). hCD36-AdV5 transduction into WT mice was also associated with reduced AdV5-GFP infectivity in mice receiving various doses of hCD36-AdV5 vector (FIG. 11, C-E).

Experimental Procedure for shRNA Adenovirus In Vitro Expression

To prepare the shRNA-adenovirus system, the shRNA is cloned into the Entry vector, pENTR™/U6. The dsDNA Oligonucleotides are annealed and incubated for 5 minutes at room temperature with the linearized pENTR™/U6 vector. The mixture is transformed into One Shot™ Top10 competent E. coli. The resulting plasmid DNA can be used immediately in a transient transfection. The shRNA expressed from the U6 promoter will form a hairpin that is processed into an siRNA molecule. The shRNA is then transferred into an expression vector. An LR recombination reaction is performed between the pENTR™ U6 entry construct and pAD/BLOCK-it™-DEST to generate the pAd/BLOCK-it™ expression construct. The Purified plasmid is digested with Pac I to expose the ITRs. Then transfect the 293 producer cell line with the adenoviral expression clone, followed by harvesting the cells and preparing a crude viral lysate. Amplify the adenovirus infecting 293A producer cells with the crude viral lysate. Determine the titer of the adenoviral stock. Then add the viral supernatant to the mammalian cell line of interest followed by assaying the target gene knockdown.

### Example 2

The results of the experiments discussed in this Example demonstrate that human SR-BIII function as viral co-receptors mediating both viral infectivity and their clearance.

### Materials and Methods

### Reagents

All media, serum preparations, cell trackers and reactive fluorescent dyes and antibiotics were obtained from Thermo Fisher Scientific. The anti-CD36 monoclonal antibody FA16 was purchased from Abcam Inc. (Cambridge, MA) as well as provided by Abruka, LCC, Estonia. Anti-hSR-BI and anti hSR-BII antibody were custom made utilizing C-terminal 15-amino acid peptides. Human HDL and LDL were isolated as reported previously.

### Cell Cultures

hSR-BI, hSR-BII, hCD36 overexpressing and mock-transfected HeLa and HEK293 cells were reported previously. HeLa (Tet-off) cells (Clontech), hCD36-overexpressing HeLa cells, as well as the human embryonic kidney cell line HEK293 (ATCC, Manassas, VA), both wild type and SR-B/II and CD36-overexpressing, were grown in Dulbecco's modified Eagle's medium (DMEM) supplemented with 10% fetal calf serum (FCS), 2 mM glutamine, 100 IU/ml penicillin, 100 μg/ml streptomycin, and 100 μg/ml G418 at 37° C. in 5% CO₂ humidified atmosphere.

Pseudo lentivirus-based particles/vectors (ppLV), AdV5 (cat #7006), AAV2 (cat #7004) and AAV8 (cat #7061) were purchased from Vector Biolab, ppLV-GVSV, ppLV-SARS-CoV2-S and ppLV-BALB were prepared following SBI Inc guidelines. Briefly, HEK 293T cells were simultaneously transfected with LV plasmid, packaging mixes plasmids: pPACK-SPIKE SARS-CoV-2 “S” Pseudo type Lentivector Packaging Mix for “Spike” pseudo typed lentiviral particles (cat #CVD19-500A-1), pPACKH1 HIV Lentivector Packaging Kit for VSV-G-pseudotyped lentiviral particles (cat #LV500A-1) and pPACK-BALD Envelope Protein-free Lentivector Packaging Mix for BALD-pseudotyped lentiviral particles (cat #LV550A-1).

### Reporter Gene Transduction in Culture

Adenovirus V5 expressing eGFP, “Spike” pseudo typed lentiviral particles, VSV-G-pseudo typed lentiviral particles, BALD-pseudo typed lentiviral particles, GFP-AAV2 and GFP-AA8 (1×106-1×1010 PFU) were incubated for 2 hours on ice (binding) or at 37° C. (uptake: binding plus internalization) followed by 48-hour chase in a virus-free media (DMEM, 10% FCS) with various cultured HeLa cells with subsequent assessment of cell fluorescence using multilabel counter (Victor 3), fluorescent microscopy (Zeiss, OptiMax 7) or by confocal microscopy as previously described.

### In Vivo Studies

WT, CD36 KO, SR-BI/II KO, hCD36, hSR-BI and hSR-BII transgenic mice were inoculated retro-orbitally (R/O) IV with 4×10⁷ PFU/g of GFP-AdV5 or 4×106 PFU/g of ppLV-SARS-COV2-S. 22 hours later, mice were inoculated with 30 μg/g of Alexa 568/633-HDL to distinguish LSEC cells and AfV5/SPIKE-ppLV mediated transduction. All animal studies were approved by the Institutional Animal Care and Use Committee of the National Heart Lung and Blood Institute under protocols H-0050R2, H-0050R4 and H-0100R3.

### Fluorescent-Labeled Ligand Uptake and Viral Transduction Analyses by Reporter Gene in Cell Culture

Adenovirus V5, native and oxidized lipoproteins and BSA were conjugated with Alexa 488/568, using a protein labeling kit (Invitrogen) following the vendor's instructions. HeLa cells, NPLCs and hepatocytes were incubated with Alexa488-AdV5 for 1 hour, followed by pulse-chase incubation with Alexa 568-Transferrin or red Lysotracker, fixed with 3.7% paraformaldehyde and stained for CD36. Images (live and fixed) were analyzed using confocal microscopy. In some experiments HeLa cells were incubated with fluorescent-labeled ligands at 37° C. for 1 hour and then washed extensively with phosphate-buffered saline (PBS), detached with Cellstripper dissociation solution (Mediatech, Herndon, VA), fixed with 4% paraformaldehyde, and analyzed by a fluorescence-activated cell sorter (FACS, model A; Hitachi). Alternatively, cell associated fluorescence was measured utilizing a Victor 3 fluorimeter (PerkinElmer). For viral transduction assessment, preparations of adenovirus V5, expressing luciferase, GFP or mCherry reporters (1×108-1×106 P FU), were incubated for 24-48 hour with various cultured cells including HeLa, HEK293, HEP, LSEC and HEP-LSEC co-culture, and the viral infectivity was analyzed by Pierce™ Firefly Luciferase Flash Assay Kit (cat #16175, Thermo-fisher), fluorimentry (Victor 3 fluorimeter), fluorescent microscopy (Zeiss, OptiMax 7) or by confocal microscopy (Zeiss 780 confocal system).

### In Vivo Studies

For transduction experiments, WT, hSR-BI, hSR-BII, SR-BKO and CD36-KO mice were inoculated retro-orbitally (R/O) IV with AdV5 (4×107 PFU/g) or SPIKE-ppLV (106 PFU/ml). AdV5/SPIKE-ppLV transduction/infectivity was analyzed by GFP, CFP and luciferase reporter expression after IV injection of AdV5 or AAV2/8 (Vector Biolabs). Twenty-two hours later, mice were further IV inoculated with 30 μg/g of Alexa 568 or Alexa 633-HDL (red), and two hours later, mice were anesthetized, sacrificed, perfused through the exterior vena cava to wash out free unbound ligands. In separate experiments testing effects of CD36 AdV5 vector driven GFP expression, mice were IV injected with 4×107 PFU/g of correspondent vectors and 24 hours later, the GFP-AdV5 infectivity was estimated by GFP reporter expression analyses as shown above. All animal studies were approved by the Institutional Animal Care and Use Committee of the National Heart Lung and Blood Institute under protocols H-0050R2, H-0050R4 and H-0100R3.

### Assessment of GFP Expression Levels in Mouse Tissues by Fluorometry and Western Blotting Analyses

Samples of excised tissues were quick-frozen on dry ice and stored at −80° C. The tissues samples were weighed, resuspended in 1:10 w/v of Tissue Protein Extraction Reagent I (Thermo Fisher Scientific) and disrupted using a chilled glass-Teflon homogenizer. The resulting lysates were centrifuged at 10,000×g for 10 minutes to pellet tissue debris. The supernatants were collected, and fluorescence intensity in the supernatants was measured using a Victor 3 multilabel counter. For Western Blotting assay, aliquots of liver supernatants were mixed with 2× sample buffer (Life Technologies), heated at 95° C. for 5 min and electrophoresed by reducing SDS PAGE, using 10% Tris-Glycine gels. Following protein transfer from gels to nitrocellulose membranes, GFP blots were analyzed using the anti eGFP monoclonal antibody (F56-6A1.2.3, Thermo Fisher Scientific) and Alkaline Phosphatase Chromogenic Substrate (Life Technologies, cat. #WP20001).

Corresponding levels of SR-BI/II and j-actin were also assessed by Western Blotting using mCD36 antibody (cat. #AF2519, R&D Systems) and j-actin antibody (cat. #4967, Cell Signaling Technology).

Total RNA Isolation and Quantitative Real-Time PCR Analysis of eGFP mRNA in the Tissues of AdV5-GFP Treated Mice

For RNA isolation, tissue samples preserved in RNA-later solution were homogenized in TRIzol Reagent using Precellys 24 homogenizer (Bertin Technologies, Montigny-le-Bretonneux, France). RNAs were isolated using the PureLink RNA Mini Kit (Thermo Fisher Scientific). After the DNase treatment, RNA (2 g) was reverse transcribed using a TaqMan Reverse Transcriptase Reagent Kit. Real-time qPCR assays were performed on a StepOne Real-Time PCR System (Applied Biosystems), using 40 ng cDNA per reaction. TaqMan Gene Expression assays for eGFP were designed by Thermo Fisher Scientific TaqMan Assay custom design service. Relative levels of gene expression were measured by the comparative CT method with mouse GAPDH (TaqMan Assay ID number Mm03302249_m1, Thermo Fisher Scientific) as a reference gene. eGFP gene expression results were analyzed using the 2−ΔΔCT formula and presented as normalized fold changes, compared to corresponding non-treated controls.

### Electron Microscopy

Confluent monolayers of HeLa cells were incubated with AdV5-LUC at 10-8 PFU/ml for 2 hours in a CO2 incubator. After three washings with ice-cold PBS, cells were fixed in PBS-buffered 2.5% glutaraldehyde (PBSG), scrubbed into the PBSG followed by double fixing in PBSG and osmium tetroxide (0.5%), dehydrated, and embedded into Spurr's epoxy resin. Ultrathin (90-nm) sections were double stained with uranyl acetate and lead citrate and viewed in a Philips CM10 transmission electron microscope (TEM) (Philips Electronics, Mahway, NJ).

### Cellular Cytotoxicity Assays

Cells were aliquoted into ACEA 96-well e-plates and allowed to sediment at room temperature for 30 min. The plates were then inserted into analyzing slots of the Real-Time Cell Analyzer (RTCA, ACEA Biosciences, San Diego, CA). Plates were cultured for 24-96 h until cells (various genetically modified HEK 293 cells) reached confluency and then, AdV5 was added at a concentration of 1×108 PFU/ml. Cell layer resistance was analyzed for the next 1-72 h. AdV5-induced cytotoxicity was measured using CytoTox 96® Non-Radioactive Cytotoxicity Assay (Promega, cat #G1780) according to the manufacturer's protocol. 10 ul of 10× lysis solution (per 100 ul of media) was added to the positive control wells to generate maximum LDH release. To calculate percent cytotoxicity for each experimental well, the following formula was used: Percent cytotoxicity=100× Experimental LDH Release (OD490)/Maximum LDH Release (OD490).

### Statistical Analyses

All data are expressed as mean values±standard deviation (SD). Differences between groups were analyzed by the Student's t-test, p<0.05 was considered significant.

### Results

The results of the experiments in this Example identify SR-B receptors, including CD36, SR-BI and SR-BII, as novel adenoviral/AAV/SARS-CoV-2/VSV receptors/coreceptors mediating viral cytotoxicity and gene transduction/infectivity in epithelial cell lines but also acting to decrease parenchymal transduction/infectivity when these class B scavenger receptors are located in the reticular endothelial system, namely, in specialized tissue endothelium such as LSEC.

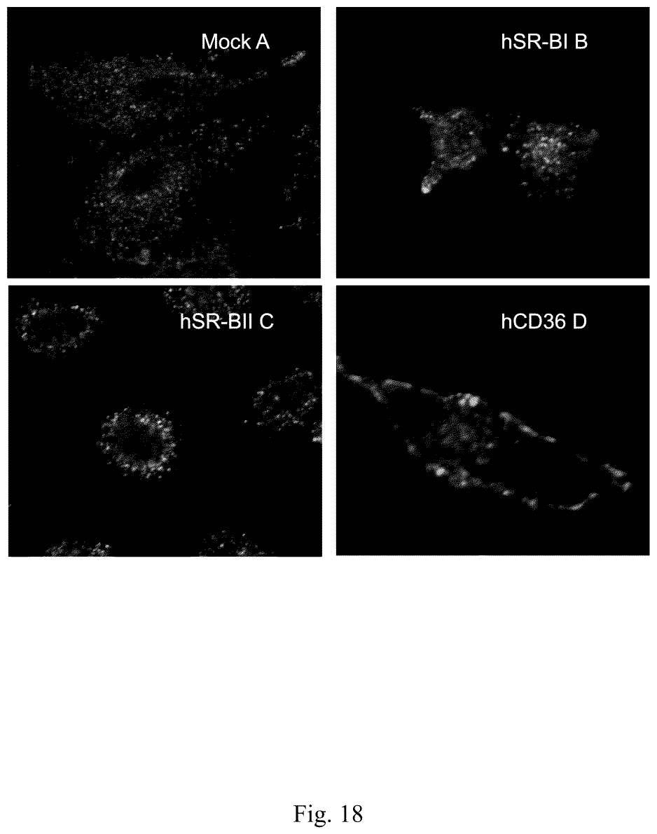

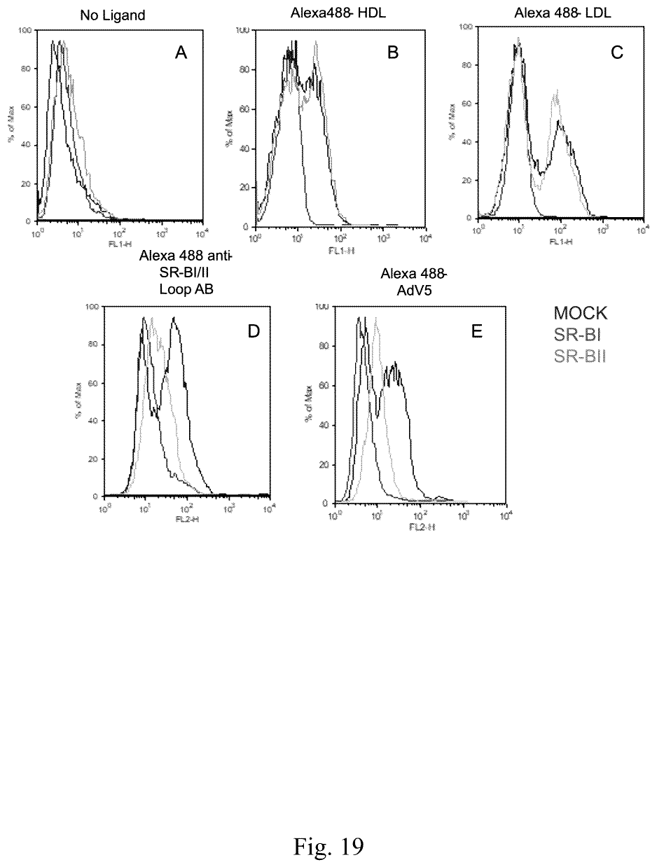

It was found that that Alexa 488-labeled AdV5 uptake was increased in HeLa cells stably transfected with hCD36, hSR-BI and hSR-BII. This approach was validated by similarly observing an increased uptake of two well established SR-B ligands, LDL and HDL, as well as using FACS analyses to demonstrate increased binding of a MoAB raised against extracellular loop of SR-BI/II domain, (FIGS. 18, 19). Data on CD36 ligands and AdV5 uptake have been similarly evaluated (data not shown).

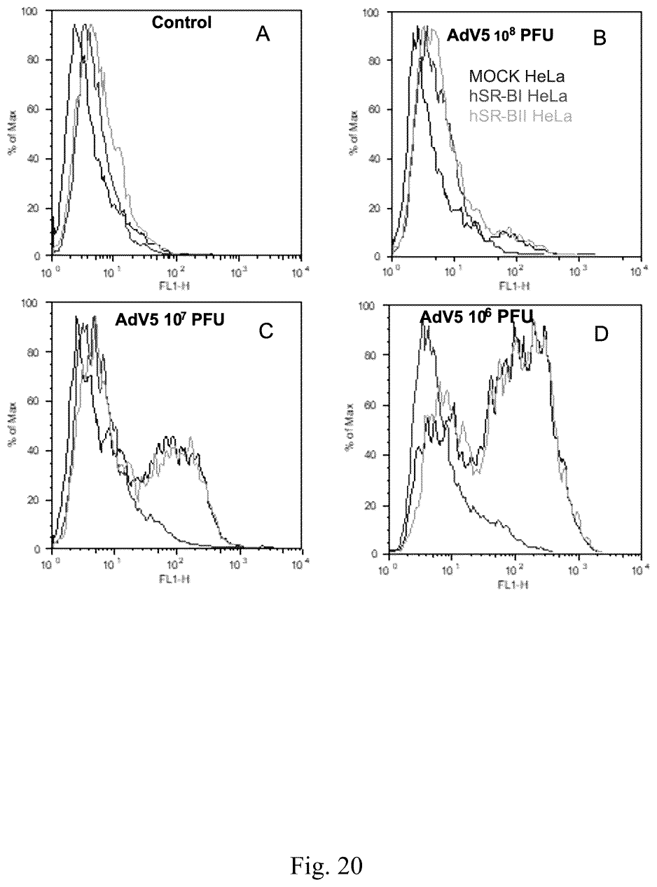

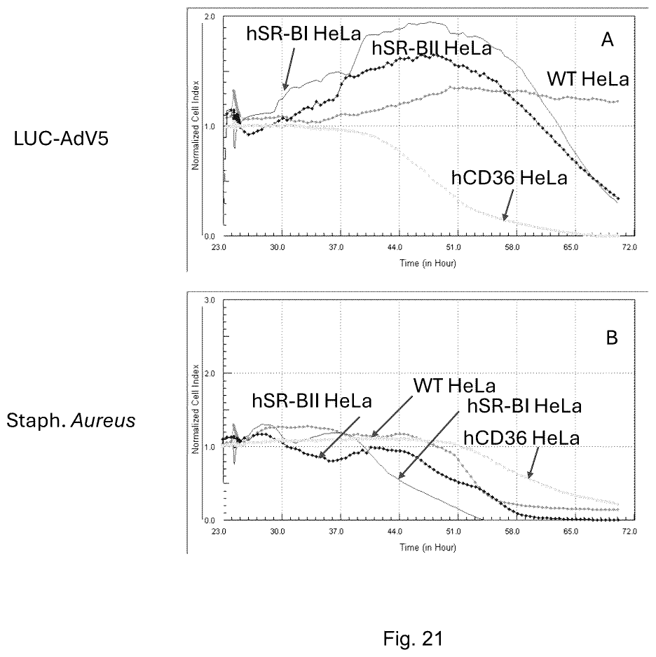

Additionally, AdV5 binding and uptake were tested utilizing GFP- and LUC-reporter expressing AdV5, that-was incubated with CD36- and Mock-HeLa cells at 37 C for 2 hours, washed and followed by further incubation in virus-free media. This approach further confirmed that all SR-B receptors including CD36, SR-BI and SR-BII are binding and endocytic receptors for AdV5, which can also productively sustain AdV5 infectivity and induce toxicity in epithelial cell lines (FIGS. 20, 21). However in vivo toxicity was not different between WT and SR-B KO mice as measured by AST and LDH plasma levels and histology.

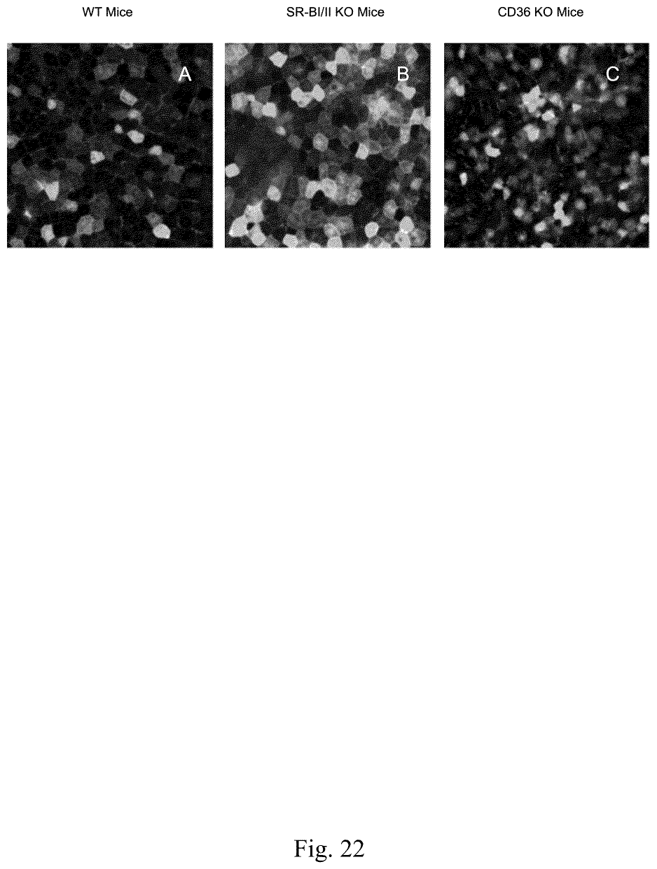

Since clearance of bloodborne AdV5 as well as other viruses involved LSEC, thus protecting hepatocytes, we analyzed GFP-AdV5-induced infectivity in vivo and have found that it effectively infected mostly hepatocytes (FIG. 22). It is known that hepatocytes have high expression levels of CAR, a recognized receptor mediating AdV5 infectivity, while expressing CD36 and SR-BUII at lower levels. Endothelial cells express high levels of CD36, SR-BUII, CD46 and other scavenger receptors, and less of CAR, which makes them less sensitive toward AdV5-induced infectivity and transduction.

To investigate the roles of SR-B in AdV infectivity and transduction in vivo, CD36KO, SR-BI/IIKO and WT mice were infected with GFP-AdV5 and sacrificed 24 hours later (FIG. 22). As seen in a set of confocal liver images a slight increase in parenchymal (epithelial) cell infectivity measured by GFP transduction was found in CD36 KO mice (FIG. 22, C). A much more visible increase was observed in the liver of SR-BUII KO mice (FIG. 22, B), where the number of GFP positive hepatocytes was increased more than 10-fold, compared to WT mice (FIG. 22, B).

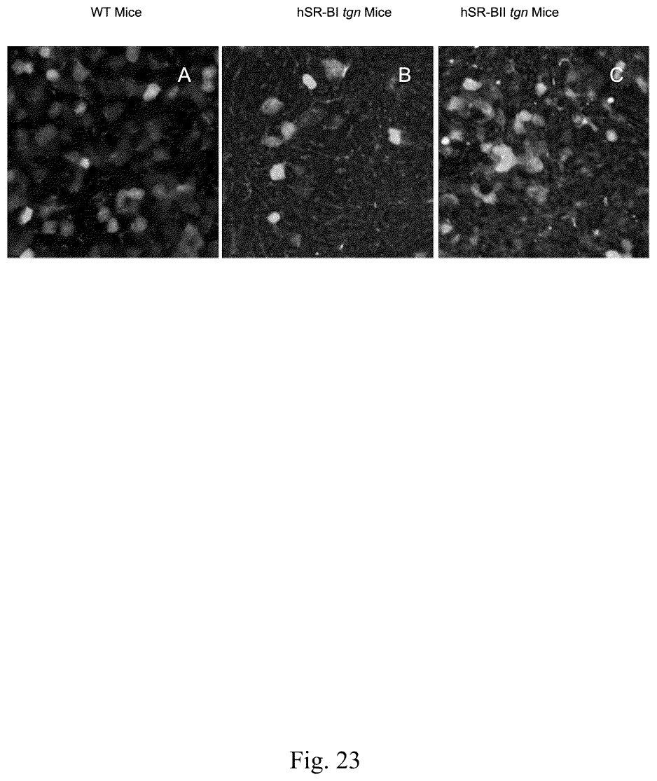

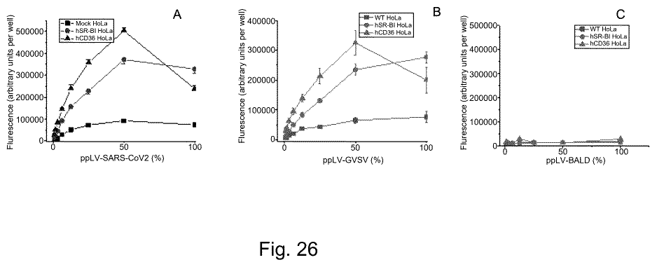

An antiviral protective role of SR-BUII was also confirmed by using SR-BI and SR-BII transgenic mice, described previously, that demonstrated enhanced antiviral defense when assessed by AdV5-GFP transduction (FIG. 23B, C). Transgenic SR-BI and SR-BII mice demonstrated increased Alexa 633-HDL uptake, confirming the expressed functional effect of SR-BUII transgenes (FIG. 26). Simultaneously, hSR-BI and hSR-BII expression drastically reduced GFP reporter expression induced by GFP-AdV5 infection (FIG. 23, B, C).

To further confirm this antiviral protective role of SR-BI, AdV5-mediated SR-BI transduction was performed in SR-BI/BII KO mice, followed by IV injection with GFP-AdV5 after 48 hours, with subsequent analysis of the livers utilizing confocal microscopy. SR-BI transduction restored SR-BI expression and increased Alexa 568-HDL uptake, demonstrating this expected functional effect of SR-BI transduction (FIG. 26). Simultaneously, SR-BI expression drastically reduced GFP reporter expression induced by GFP-AdV5 infection.

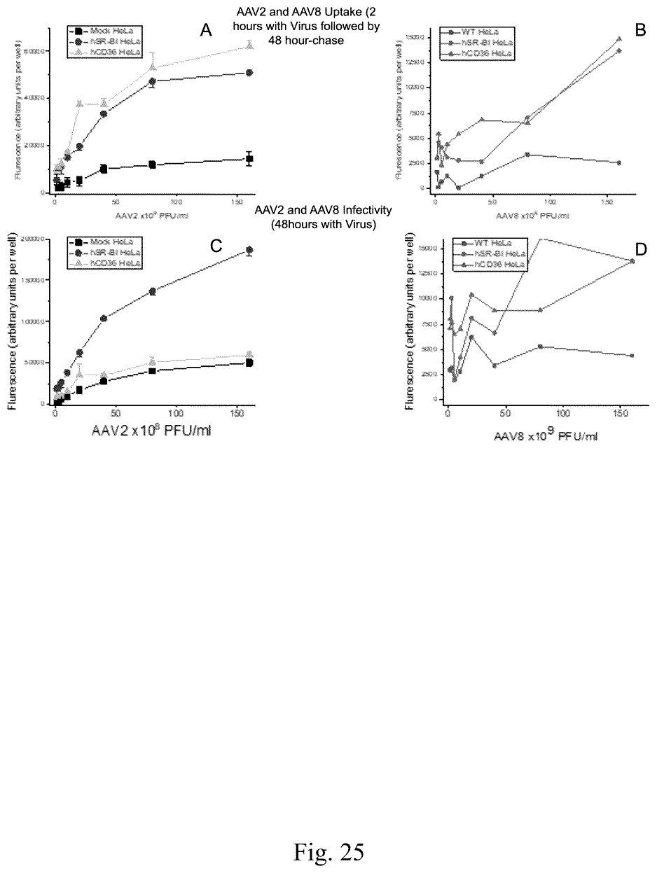

To establish a more general role of SR-B in antiviral recognition and antiviral defense, WT, CD36 and hSR-BI overexpressing HeLa cells were incubated with various viruses and their GFP reporter expressing pseudo typed analogues, including AAV2, AAV8 (FIG. 25) as well as ppLV-GVSV, ppLV-SARS-COV2-S and ppLV-BALD (FIG. 26). These data demonstrate that SR-BI and CD36 are receptors/co-receptors mediating binding and transduction of AAV viruses and pseudo typed analogues of VSV and SARS-COV2.

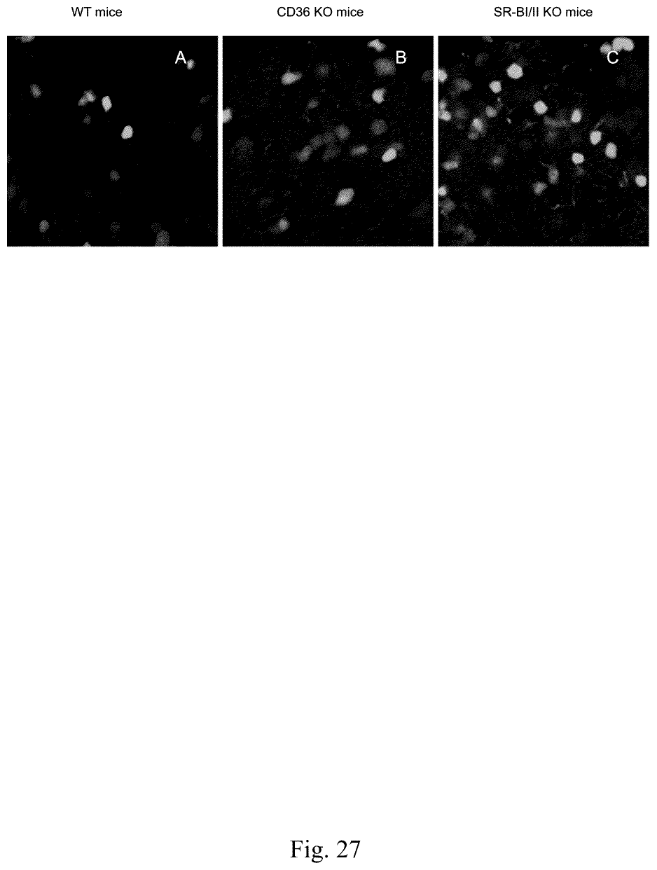

To investigate the role of SR-B in ppLV-SARS-COV2-S transduction in vivo, CD36KO, SR-BI/IIKO and WT mice were infected with SARS-COV2 pseudo typed analogues with GFP expressing reporter. The animals were sacrificed 24 hours later and analyzed utilizing confocal microscopy (FIG. 27). The data reveal a moderate increase in parenchymal (epithelial) cell infectivity as measured by GFP transduction in CD36 KO mice (FIG. 27, B), while a much greater difference was observed in the liver of SR-BI/II KO mice (FIG. 27, C), where the number of GFP positive hepatocytes increased more than 3-4-fold. Thus, this result demonstrates that rather than being a gateway for infection, SR-BI and CD36 are the part of antiviral defense in vivo, not only for bloodborne AdV5, but also for SARS-CoV2 pseudo typed analogues with a GFP expressing reporter.

In conclusion, this Example for the first time demonstrates that SR-B are not only an anti-bacterial host defense mechanism mediating clearance of gram-negative bacterial pathogens and signaling of PAMPs and DAMPs, but also an anti-viral defense mechanism protecting parenchymal cells of various tissues from a direct interaction with virus, mediating viral clearance and metabolism in SR-B expressing cells of the RES. These findings can help to develop new antiviral therapies. For example, one approach could involve using the agents capable to direct viruses to SR-B-mediated viral clearance/neutralization pathways.

### Example 3

The results of the experiments discussed in this Example demonstrate that SR-BI is a coreceptor for Mouse Hepatitis virus (MHV), facilitating MHV uptake and amplification in vitro. In brief, the data demonstrate that SR-BI can enhance MHV transduction, suggesting that it can function as a co-receptor of the principal MHV receptor CEACAM1, facilitating viral entry by mediating initial binding and attachment to the host cell surface.

### Materials and Methods

### Reagents

All media, sera, antibiotics, and all reagents used for RNA isolation, reverse transcription, and real-time PCR were purchased from Thermo Fisher Scientific (Waltham, MA, USA). Recombinant mouse macrophage colony-stimulating factor (M-CSF) was purchased from Sigma-Aldrich (St. Louis, MO, USA). The anti-SRBI blocking antibody was purchased from Novus Biological. Human HDL was isolated as reported previously.

### Cell Cultures

The following primary cultures and cell lines were used in this study:
1) Mouse liver cells BNL CL.2 (epithelial cells from normal liver);
2) Mouse LR7 cell line (mouse fibroblast cells derived from L cells transfected with the MHV receptor mCEACAM1a;
3) Primary cultures of mouse hepatocytes and liver sinusoidal endothelial cells (LSEC) isolated from WT and hSR-BI/BII transgenic mice as described in Example 1;
4) Bone marrow-derived macrophages (BMDM) isolated from WT and SR-BI/BII-knockout mice. The macrophages were differentiated by culturing in RPMI-1640 supplemented with 10% fetal calf serum (FCS), in the presence of 10 ng/mL of mouse M-CSF for 7-10 days.

### Assessment of MHV Transduction

The MHV-A59 preparations used in various doses (10−3-10−7 PFU/ml), depending on cell type, were incubated for 2 hours on ice (binding) or at 37° C. (uptake) followed by an 18-hour chase in a virus-free media (DMEM, 10% FCS) at 37° C. with various cultured cell lines or primary cultures. Subsequent assessment of cell-associated fluorescence used a multilabel counter Victor 3.

Total RNA Isolation and Quantitative Real-Time PCR Analysis of MHV Membrane M-Protein and GFP mRNA

Following MHV treatment, total mRNA was isolated from cells using Trizol Reagent and the PureLink RNA Mini Kit (Thermo Fisher Scientific) according to the manufacturer's protocol. After DNase treatment, RNA (2 g) was reverse transcribed using a TaqMan Reverse Transcriptase Reagent Kit. Real-time qPCR assays were performed on a StepOne Real-Time PCR System (Applied Biosystems), using 40 ng cDNA per reaction. TaqMan Gene Expression assays for GFP and MHV M-Protein were ordered from Thermo Fisher Scientific TaqMan Assay custom design service. Relative gene expression levels were measured by the comparative CT method with mouse GAPDH (TaqMan Assay ID number Mm03302249_m1, Thermo Fisher Scientific) as a reference gene. GFP and MHV M-protein gene expression results were analyzed using the 2−ΔΔCT formula and presented as normalized fold changes, compared to corresponding non-treated controls.

### Results

hSRBI Increases MHV Infectivity in Mouse Hepatoma BNL CL.2 and LR7 Fibroblast Mouse Cell Lines

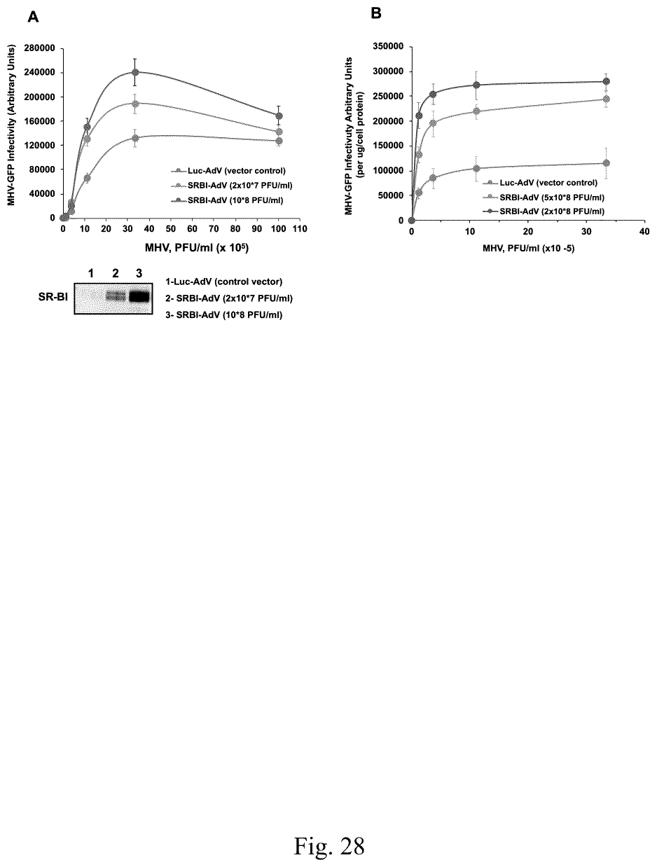

To evaluate the SR-BI role in MHV-infectivity, transient transfection of two different MHV-permissive mouse cell lines, mouse hepatoma BNL CL.2, and fibroblast LR7, was performed with hSR-BI adenoviral vector. The data reveal that that both cell line types as well as hepatocytes (FIG. 28A) and fibroblasts (FIG. 28B) overexpressing hSRBI, demonstrated about 1.5-2 times higher (depending on the expression level of hSRBI) MHV transduction levels compared to control ADV-Luc vector-transfected cells (FIG. 28).

### MHV Uptake and Infectivity are Elevated in SR-BI-Transfected Vs Vector-Transfected Mouse Hepatoma BNL CL.2.

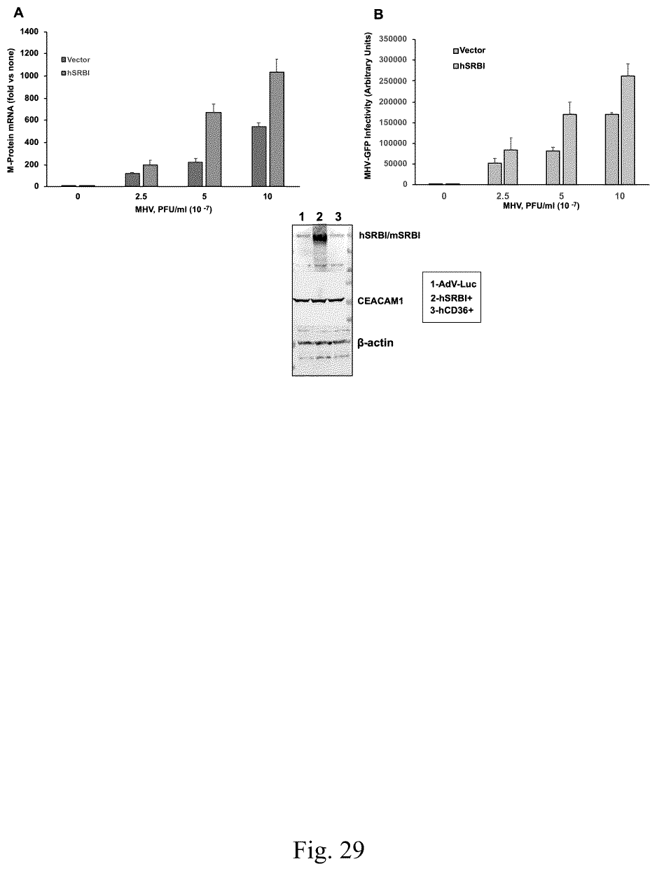

To further analyze the specific steps of SR-BI involvement in MHV infectivity, MHV uptake and transduction levels in vector- and hSR-BI-transfected mouse hepatoma BNL CL.2 cells was assessed. After a 2-hour incubation at 37° C. (uptake) with increasing doses of MHV, between 2-2.5 times higher levels of M-protein mRNA in cells expressing hSR-BI was observed, as compared to control Luc-AdV-transfected cells (FIG. 29A). Simultaneously, MHV transduction levels, measured after 2 hours of incubation with the virus followed by 18 hours incubation in serum-free media w/o virus, were also elevated in SR-BI-expressing cells, though to a lesser extent, approximately 1.5- to 2-fold (FIG. 29B).

MHV Infectivity Levels are Higher in Hepatocytes and Liver Sinusoidal Epithelial Cells Isolated from hSRBI (CLA-1) Transgenic Mice Compared to WT Cells

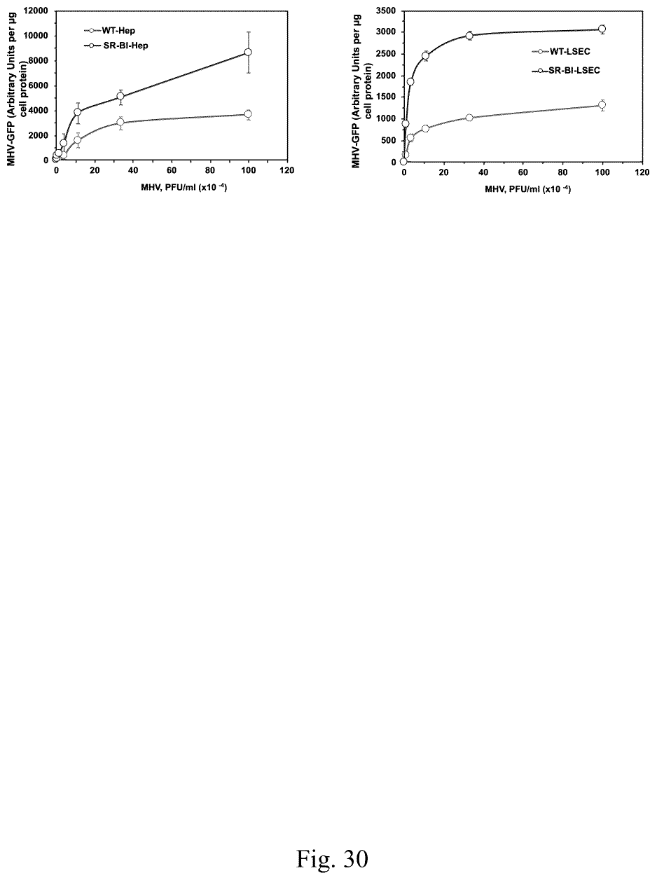

Next, another cell model responsive to MHV, primary cell cultures of hepatocytes and NPLC, was used, and the data reveal that MHV infectivity was markedly, 2.5-3 times higher, in both types of liver cells isolated from hSR-BI transgenic mice compared to wild-type control cells (FIG. 30).

### MHV Infectivity is Reduced in SRBI/BII-Deficient Mouse Bone Marrow-Derived Macrophages

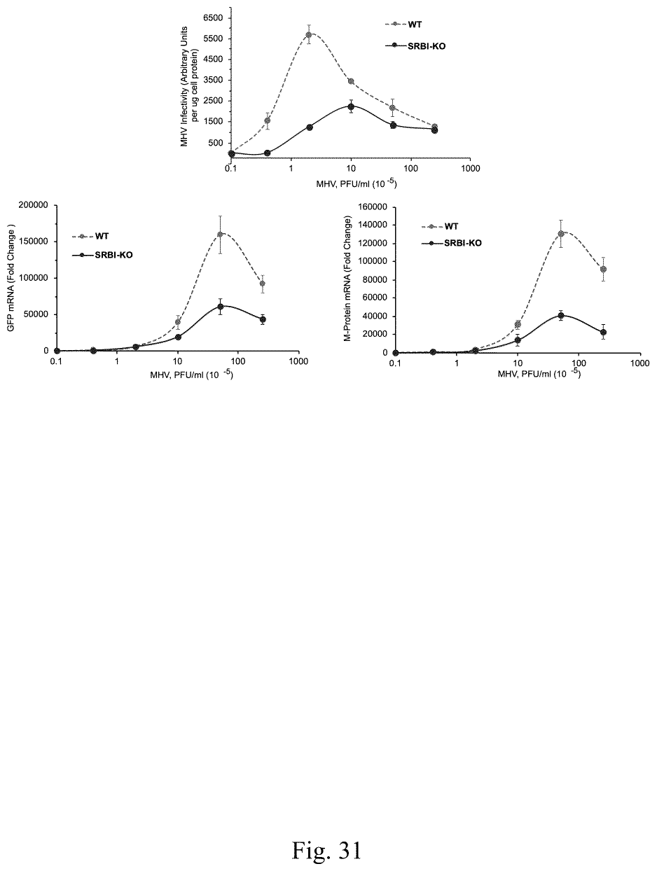

To obtain additional evidence of the SR-BI's role in MHV infectivity, the GFP and M-protein mRNA expression levels in bone marrow-derived macrophages (BMDM) from WT and SR-BI/BII-KO mice following 2 hrs of MHV treatment was quantified. The data, presented in FIG. 31, demonstrate that SR-BI-deficient BMDM had markedly lower mRNA expression of both viral proteins, GFP (panel B) and M-protein (panel C), compared to control macrophages isolated from wild-type mice. The MHV transduction level (FIG. 31A), assessed by the intensity of cell-associated GFP signal, was also significantly reduced in BMDM from SR-BI/BII-KO mice.

### Apo AI-Mimetic SAHPs and Anti-SR-BI Blocking Antibody Inhibit MHV Infectivity in Mouse Hepatoma BNL CL.2.

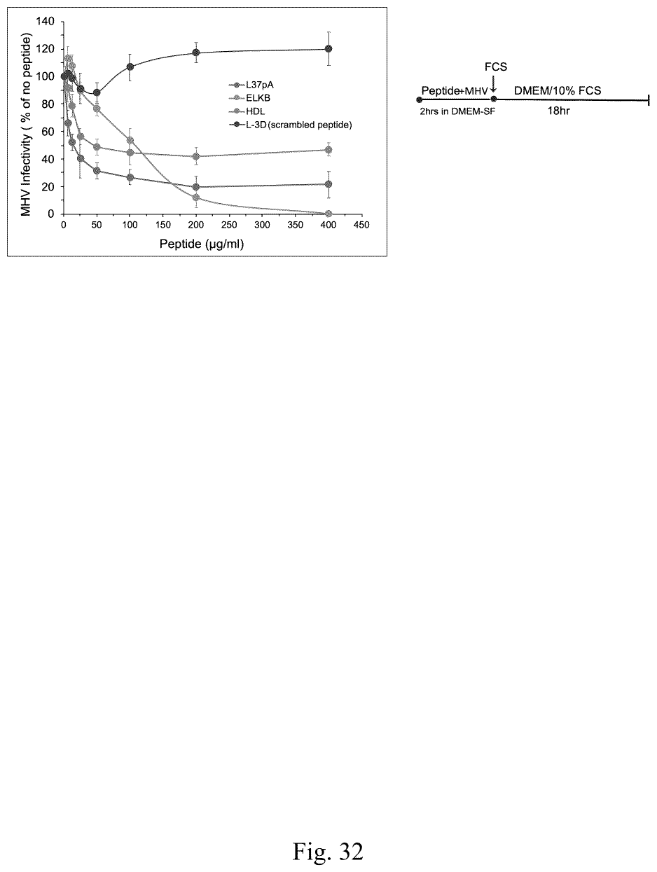

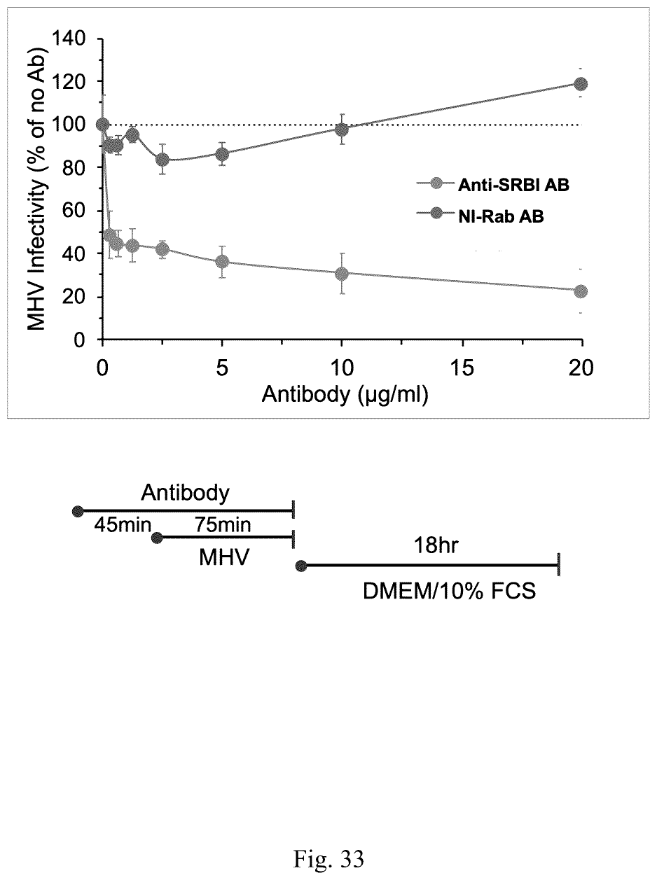

Next, the ability of SAHPs, known SR-BI agonists, to inhibit MHV uptake and infectivity in mouse hepatoma cells, was assessed. As demonstrated in FIG. 32, L37 pA and ELKB efficiently inhibited MHV infectivity in a dose-dependent manner, while the scrambled L3D peptide did not have any blocking activity. When the SR-BI blocking antibody (raised against the receptor-binding extracellular loop) was preincubated with the cells before the 2-hr incubation with MHV, significant (70-80%) inhibition of MHV transduction in the mouse hepatoma cell line also was observed (FIG. 33).

### Example 4

The results of the experiments discussed in this Example demonstrate the effect of class B scavenger receptor expression and treatment with amphipathic helical peptides on SARS-CoV-2 infectivity.

### Materials and Methods

### Viral Infectivity Assay in Calu3 Cells

Calu3 cells (human airway epithelial cell line) were maintained in RPMI-1640, 10% FBS, and 1% Glutamine. For viral infectivity assays, Calu3 cells were seeded at 3×10^{5/12} well plates and cultured at 37° C. After 48 h incubation, the culture medium was replaced with RPMI-1640+2% FBS and cells were infected with SARS-CoV-2 delta at MOI=0.1. At 24 h post infection, 2 aliquots of each well medium at 110 ul/aliquot were collected and virus in the media was titered by plaque assay.

### Calu-3 Transfection in Calu3 Cells

For siRNA knockdown treatment, siRNAs (20 nM) were transfected into Calu3 cells by reverse transfection method using Lipofectamine RNAiMax (Thermol Fisher) according to manufacturer's instruction when Calu3 cells were seeded into 12 well plates at 3×10⁵/well. After 48 h incubation at 37° C., the cell culture media were replaced with RPMI-1640+2% FBS and the cells were infected with the virus as described above.

For gene over-expression, Calu3 cells were seeded at 3×10^{5/12} well plates and cultured at 37° C. After 24 h incubation, gene encoding plasmids were transfected into Calu3 cells using X-tremeGene HP (Sigma) according to the manufacturer's instructions. After 24 h post transfection, the cell culture media were replaced with RPMI-1640+2% FBS and the cells were infected with the virus as described above.

### Amphipathic Helical Peptide Treatment of SARS-CoV-2 Virus Using Calu3 Cells

Calu3 cells were prepared and treated with virus as described above. 30 minutes prior to treatment with SARS-CoV-2 delta, L-37 pA, ELKB and L3D-37 pA were added at various concentrations and maintained during the entire incubation. Two aliquots of each well medium at 110 ul/aliquot were collected and virus in the media was tittered by plaque assay.

### Effect of Class B Scavenger Receptor Expression In Vivo on Mouse SARS-CoV-2 Induced Mortality

SR-BI KO (background Taconic C57BL/6), heterozygotes and control (Taconic C57BL/6) mice as well as CD36 KO (background Jackson Lab C57BL/6) and control (Jackson Lab C57BL/6) were treated with mouse adapted SARS-CoV-2 (strain MA10 from bei Resources) with a dose of 1e+05 (stock titer: 2.15e6 PFU/ml, 4.31e6 TCID50/ml) instilled intranasally and followed over 14 days (for SR-BI experiment) and 10 days (for the CD36 experiment). The CD36 experiment was done for 10 days as all deaths in the SR-BI experiment occurred within the first 8 days. In addition, similar rates of mortality were noted in the SR-BI experiment (20%) and the CD36 experiment (17%) indicating the shorter period of follow-up for CD36 did not affect the results. Weights were determined every 2-4 days. Mortality was determined by either death, a weight loss greater than 25% or meeting euthanasia criteria.

### Results

To evaluate the potential contribution of class B scavenger receptors on SARS-CoV-2 infectivity, these receptors were up and down regulated in vitro by transfection using a known SARS-CoV-2 susceptible human lung cell line, Calu3.

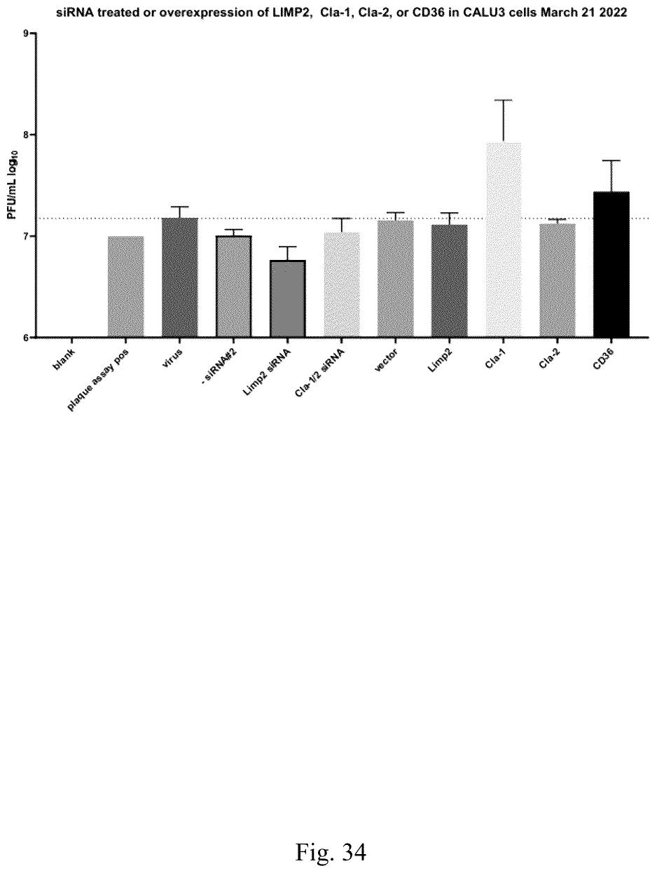

As revealed in FIG. 34, down regulation of both SR-BI/SR-BII (CLA-1/CLA-2) and LIMP2 (an intracellular class B scavenger receptor) reduced infectivity compared to the virus control, but only LIMP2 reduced infectivity relative to the siRNA control. Both CD36 and SR-BI overexpression appeared to increase infectivity. These results suggest that LIMP2 is needed for optimal SARS-CoV-2 infectivity and that decreasing LIMP2 quantity or activity may decrease SARS-CoV-2 infectivity.

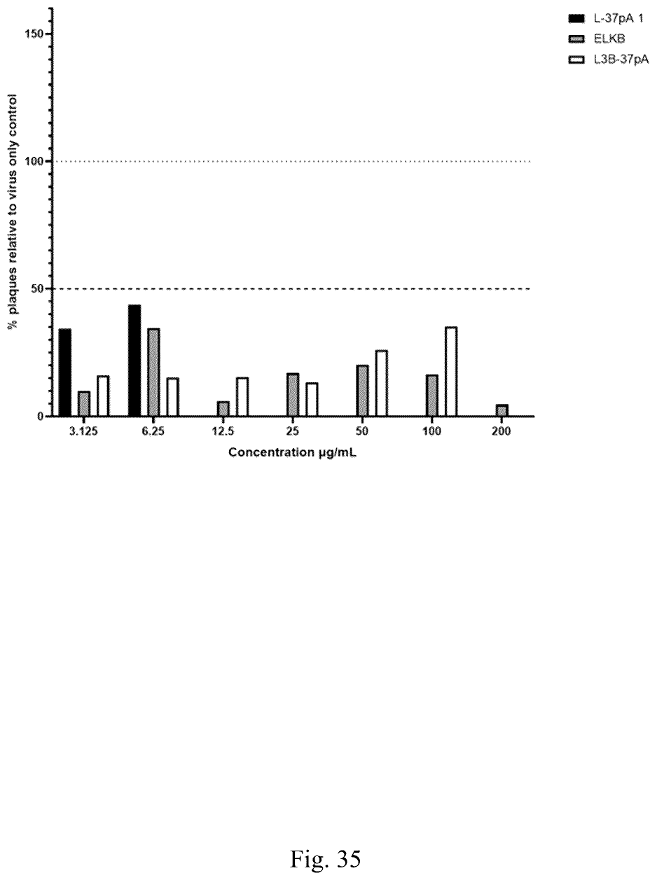

Amphipathic helical peptides including L37 pA (most potently targeting SR-BI), ELKB (most potently targeting CD36) and L3D-37 pA (scrambled control peptide) were evaluated for their ability to affect SARS-CoV-2 infectivity in CALU3 cells. As revealed in FIG. 35, L37 pA had a dose-response with complete inhibition of infectivity beginning at 12.5 ug/ml. Neither ELKB nor L3D-37 pA had a dose response and did not completely inhibit infectivity at concentrations less than 100 ug/ml, suggesting some non-specific effect contributing to infectivity inhibition.

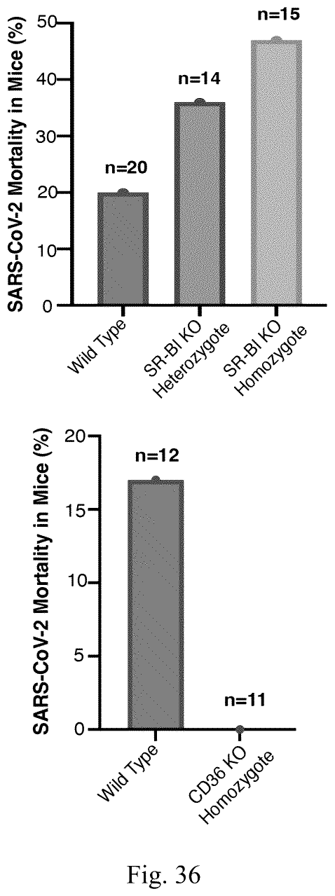

Additionally, knocking out SR-BI and CD36 in mice would affect SARS-CoV-2 infectivity was studied. In two experiments, using mouse-adapted SARS-CoV-2 virus, SR-BI KO mice, and heterozygote KO mice and controls had respective mortality rates of 47%, 36% and 20%, demonstrating a gene dosage effect and indicating a significant essential function of SR-BI in protecting against infection (FIG. 36). Of note, one of the functions of class B scavenger receptors, such as SR-BI, when expressed in liver sinusoidal endothelial cells (LSEC), is to recognize and clear various viruses. In a single experiment CD36 KO mice had no mortality compared to control mice (17% mortality). These results suggest an important function of CD36 in SARS-CoV-2 facilitating infectivity.

Overall, these in vitro and in vivo results in this Example suggest important roles of class B scavenger receptors in affecting SARS-CoV-2 infectivity. In vitro, LIMP2, SR-BI and CD36 can promote infectivity. Decreasing LIMP2 quantity or activity may inhibit SARS-CoV-2 infectivity. In vivo SR-BI appears to be most important in viral clearance and that increasing SR-B1 quantity or activity may inhibit SARS-CoV-2 infectivity, whereas CD36 appears to be most important in facilitating infectivity, and decreasing CD36 quantity or activity may inhibit SARS-CoV-2 infectivity. These differential effects result from in vivo net infectivity reflecting the contribution of class B scavenger receptor facilitated viral infectivity in susceptible cells and virus degradation mediated by liver sinusoidal endothelial cells (LSEC) that express both SR-BI and CD36. Although other viruses may have different relative results, the principal of targeting class B scavenger receptors to decrease viral infectivity is likely applicable to many viruses.

### Example 5

The results of the experiments discussed in this Example demonstrate that CD36 is a receptor for AdV5 vector, mediating AdV vector uptake and transduction as well as decrease AdV5 viral activity/increase degradation in some cells/conditions in vitro.

To investigate the role of CD36 in various stages of AdV5 vector transduction, viral binding, entry and replication were evaluated with several mouse cell lines using host cells both expressing CAR and lacking its expression.

### Materials and Methods

### Reagents

All media, serum preparations, cell trackers, reactive fluorescent dyes and antibiotics were obtained from Thermo Fisher Scientific. The anti-CD36 monoclonal antibody FA16 was purchased from Abcam Inc. (Cambridge, MA). Human HDL and LDL were isolated as reported previously. Extensively oxidized LDL (oxLDL) was prepared by incubation with 5 μM CuSO4 at 37 C for 24 has previously described. Insulin, dexamethasone, Percoll, Opti Prep, FBS were from Sigma-Aldrich.

### Cell Cultures

Stably transfected epithelial cell lines including HeLa and HEK293 cells, mock-transfected cells (Mock-HeLa and Mock-HEK) and overexpressing human CD36 cells (CD36-HeLa and CD36-HEK) were characterized previously. Cells were grown in Dulbecco's modified Eagle's medium (DMEM) supplemented with 10% fetal calf serum (FCS), 2 mM glutamine, 100 IU/ml penicillin, 100 μg/ml streptomycin, and 100 μg/ml G418 at 37° C. in 5% CO2 humidified atmosphere. NPLCs and hepatocytes were isolated from livers of CD36-deficient or wild type rats/mice using a method described previously with modifications where an Opti-Prep Density Gradient Reagent (Sigma) was used instead of Percoll for density gradient centrifugation for the isolation of NPLCs. Briefly, animals were anesthetized using ketamine/xylazine/acepromazine (80/10/0.02 mg/kg), the liver was perfused with 500 ml of Ca²⁺/Mg²⁺-free Hank's balanced salt solution (HBSS) followed by a perfusion with 15 ml of 1 mg/ml type I collagenase type 1 (Worthington, USA, cat #LS004197) in Mg²⁺-free HBSS containing 4 mM CaCl₂ at the rate of 3 ml/min. Liver cells were re-suspended in HBSS. Hepatocytes were sedimented by repetitive centrifugations at 50×g for 3 min in ice-cold HBSS. Supernatants containing sinusoidal non-parenchymal liver cells (NPLC) were collected and centrifuged at 300×g for 5 min. Pelleted NPLC were re-suspended in 17% of Opti-Prep Density Gradient Isosmotic Reagent solution in HBSS and centrifuged at 1500×g for 20 min. The NPLC, floated at the top of the gradient, were collected and pelleted by an additional centrifugation at 300×g for 5 min.

The NPLC suspension was plated in serum-free DMEM into 96-well culture plates for 15 min to allow Kupffer cells (KC) to attach and spread. Non-attached cells were removed from culture plates by extensive washing, and the KC were further cultivated in RPMI 1640 containing 10% FCS and 10 ng/mL M-CSF. Supernatants containing LSEC as well as isolated HEP were plated on collagen coated plastic plates or glass slides in William's E media containing 10⁻⁶ M dexamethasone and 10 μg/ml Insulin (WEDI). After two hours the cells were washed with PBS and further cultivated in WEDI. Additionally, we characterize a LSEC compartment of mouse liver by an imaging for Alexa 633 labeled formaldehyde-treated BSA (LSEC-specific ligand) uptake.

### Fluorescent-Labeled Ligand Uptake and Reporter Gene Transduction Induced by AdV5 in Cell Culture

Adenovirus V5, native and oxidized lipoproteins and BSA were conjugated with Alexa 488/568, using a protein labeling kit (Invitrogen) following the vendor's instructions. HeLa cells, NPLCs and hepatocytes were incubated with Alexa488-AdV5 for 1 hour, followed by a pulse-chase incubation with Alexa 568-Transferrin or red Lysotracker, fixed with 3.7% paraformaldehyde and stained for anti-hCD36 FA16 antibody. Images (live and fixed) were analyzed using confocal microscopy. In some experiments HeLa cells were incubated with fluorescent-labeled ligands at 37° C. for 1 hour and then washed extensively with phosphate-buffered saline (PBS), detached with Cellstripper dissociation solution (Mediatech, Herndon, VA), fixed with 4% paraformaldehyde, and analyzed by a fluorescence-activated cell sorter (FACS, model A; Hitachi).

Alternatively, cell associated fluorescence was measured utilizing a Victor 3 fluorimeter (PerkinElmer). For viral transduction assessment, preparations of adenovirus V5, expressing luciferase, GFP or mCherry reporters (1×10⁸-1×10⁶ PFU), were incubated for 24-48 hour with various cultured cells including HeLa, HEK293, HEP, LSEC and HEP-LSEC co-culture, and the viral infectivity/transduction was analyzed by Pierce™ Firefly Luciferase Flash Assay Kit (cat #16175, Thermo-fisher), fluorimentry (Victor 3 fluorimeter), fluorescent microscopy (Zeiss, OptiMax 7) or by confocal microscopy (Zeiss 780 confocal system). For AdV5 binding assessment, confluent cells were incubated with various concentrations of GFP-expressing AdV5 vectors (1×10⁶-1×10⁸ PFU/ml) for 2 hours on ice. Afterwards, cells were washed with ice-cold PBS three times and further incubated in basic media for 24-48 hours. GFP expression was determined as written above.

### Cross-Linking Protocol for Cross-Linking in Cell Culture:

CD36-HeLa and Mock-HeLa were grown in basic media until full confluence, afterwards cells were washed with Ca, Mg free PBS two times and further incubated with DMEM containing 2 mg/ml BSA with or without AdV5-GFP at 10¹⁰ PFU/ml for 1 hour in CO₂ incubator. The cells were washed with ice-cold PBS three times and immediately added with pH 7.3 buffered PBS/10% formalin and incubated 10 minutes with slow rotation at room temperature (RT). Cells were washed with TBS and further lysed with extraction buffer (EB, 50 mM TRIS, 1% Triton 100 added with enzyme inhibitors) 10 min at RT. Scrubbed cells and lysate were collected and further centrifuged 1000 g×10 min, cell extract was used for Cross-linking-ELISA.

### Protocol for Cross-Linking ELISA:

96-well ELISA plate were coated with anti-AdV5 mouse polyclonal antibody 5 μg/ml in 10 mM carbonate buffer pH 9.6 overnight at 4° C., washed with PBS, 0.1% Tween 20 (wash buffer-WB) 3 times for 3 minutes and block WB containing 2 mg/ml BSA for 1-2 hours at RT. Plated were washed with WB once and incubate with serial dilutions of cell extracts overnight at 4° C., wash with WB 3 times for 3 minutes at RT. Plates were farther incubated for two hours with 2-5 μg/ml biotinylated anti-CD36 antibody in WB at RT, washed with WB 3 times for 3 minutes at RT. For visualization we used an HRP-streptavidin and development substrate.

### AdV5 Activity/Degradation Assay

CD36-HeLa and Mock-HeLa were grown basic media until full confluence afterwards, cells were washed with Ca, Mg free PBS two times and further incubated with DMEM containing 2 mg/ml BSA with or without AdV5-GFP at 10×¹⁰ PFU/ml for 1 hour in CO₂ incubator. The cells were washed with ice-cold Ca; Mg free PBS three times and immediately ice-cold water was added and incubated for 10 minutes and centrifuged 1000 g×10 min. The cell extract was serially diluted into 96-well plates with cultured WT HeLa cell in 10% FCS/DMEM for 24 hours for development of GFP expression. The GFP signal was measured using a Victor 3 Fluorimeter.

### Results

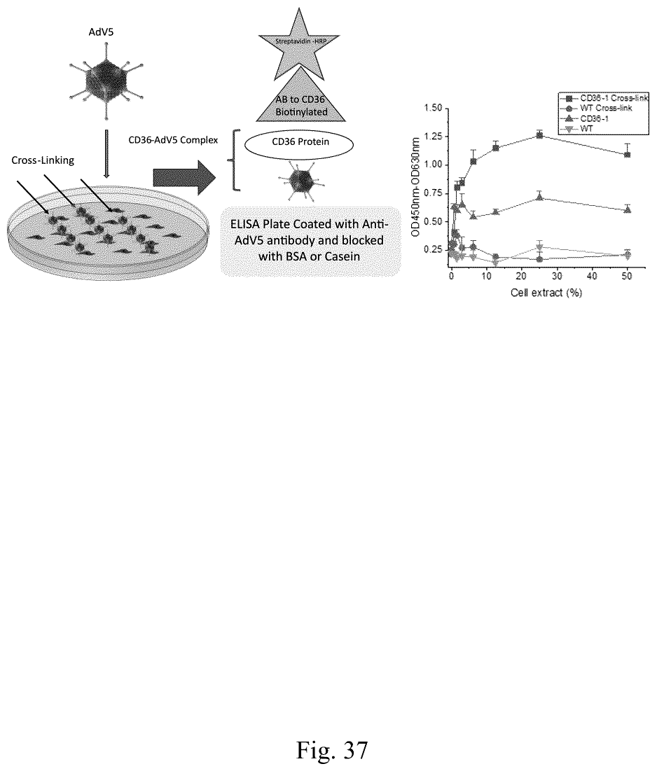

A direct interaction between adenoviral vectors with CD36, one of the SR-B family members (FIG. 37), was studied. When CD36 is overexpressed in HeLa cells (CD36-1), there is increased ADV5 binding compared to WT cells. Cross-linking makes this complex more stable than without cross linking. These data suggests that CD36 directly binds AdV5, forming a ligand-receptor complex.

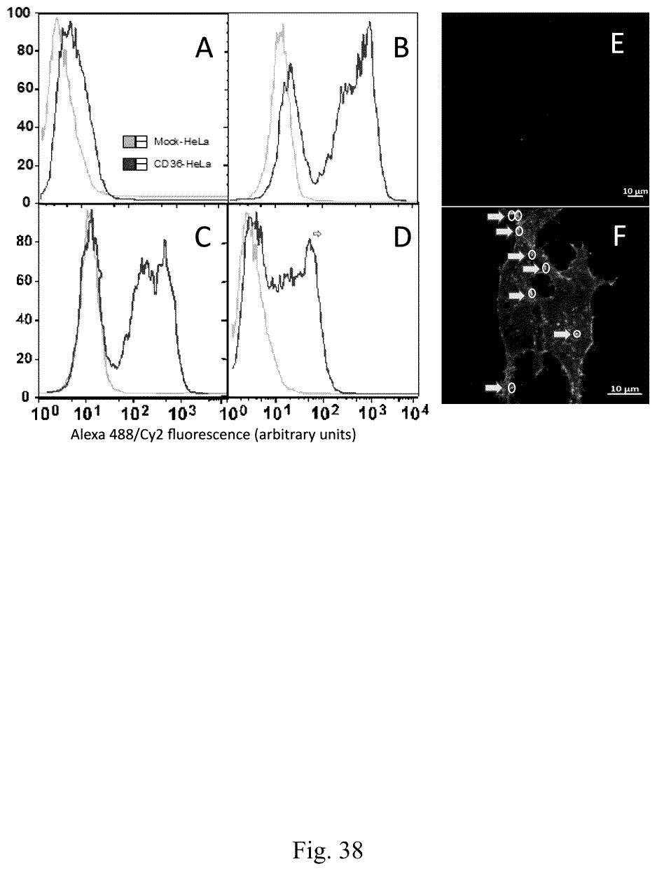

To investigate CD36's potential role as an adenoviral receptor, the uptake of Alexa 488-protein labeled AdV5 in HeLa cells stably transfected with CD36 (CD36-HeLa) was studied. Using FACS analyses, it was discovered that CD36 overexpression increased uptake not only of well-established CD36 ligands such as LDL and oxLDL, but also Cy2-AdV5 by 24-fold as judged by fluorescent peak shift at FACS graph of Cy2-AdV5 uptake in CD36-HeLa (red line) compared to a mock-HeLa control (green lane) (FIG. 38, A-D). AdV5 and CD36 are co-localized in the cells expressing CD36 (FIGS. 38, E and F). Circles demonstrate areas of colocalization.

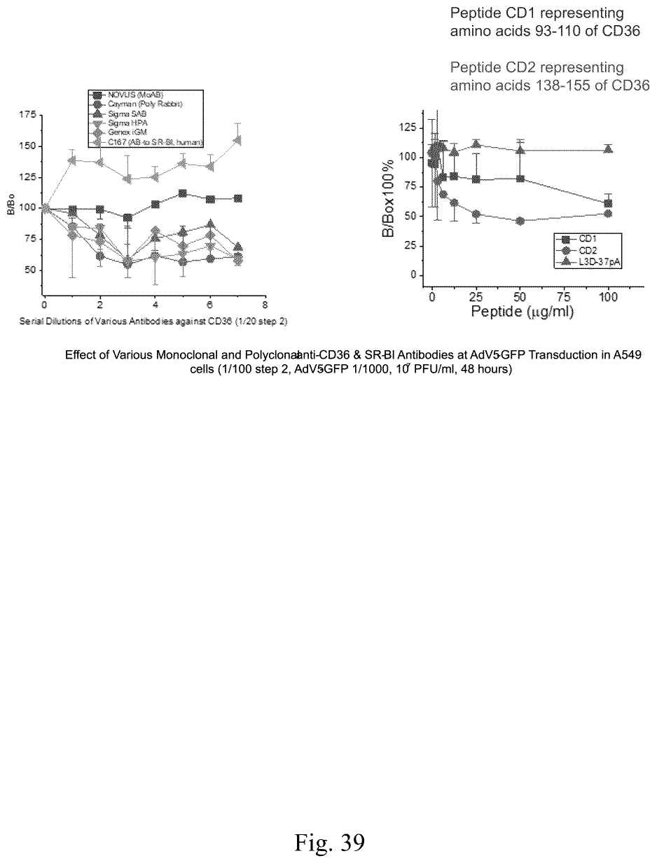

CD36-AdV5 interaction can be blocked by anti-CD36 polyclonal antibodies as well as CD36 binding domain derived synthetic peptides including CD1 and CD2 (FIG. 39).

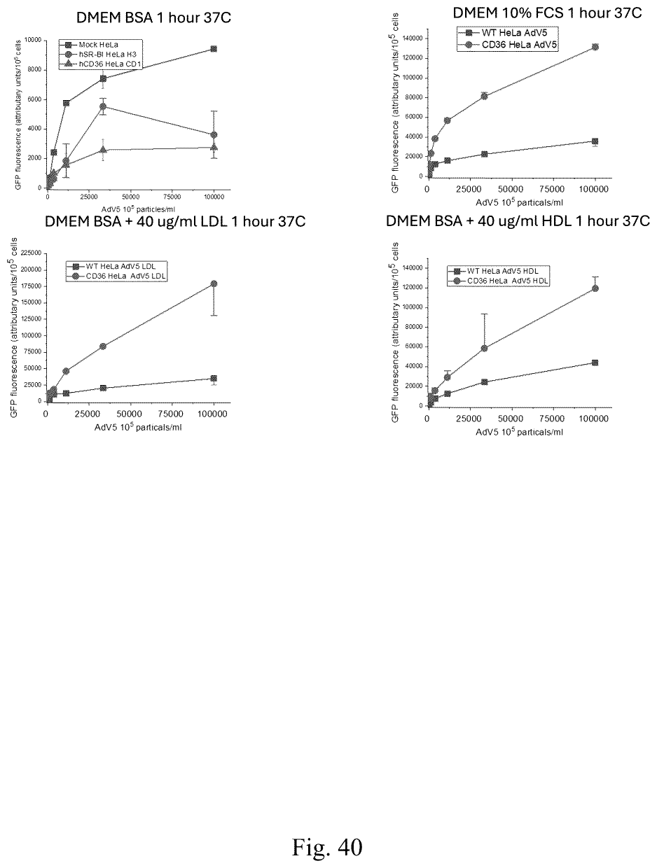

The AdV5 vector-mediated GFP gene transduction was determined to be critically dependent on serum and lipoproteins such as LDL and HDL when tested as earlier in HeLa cells (FIG. 40). In contrast to serum free AdV5 mediated transduction, where it was reduced in CD36 overexpressing cells (upper left panel), the presence of serum, HDL or LDL increased transduction indicating that AdV5-lipoprotein complexes are critical in some cell types in mediating AdV5 transduction. In the absence of serum, HEK293 were like HeLa cells with AdV5 vector-mediated gene transduction being reduced by several fold (data not shown).

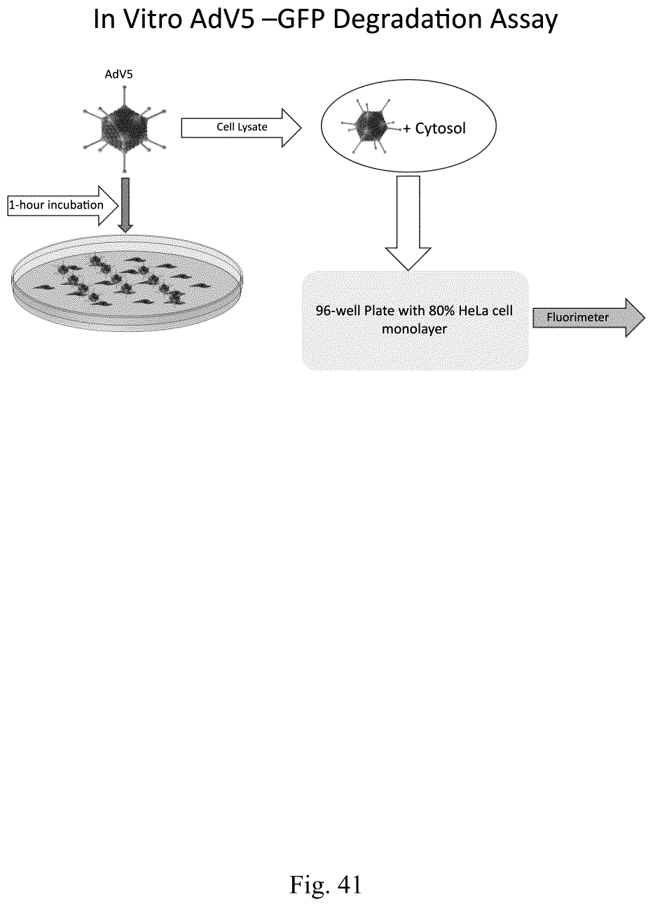

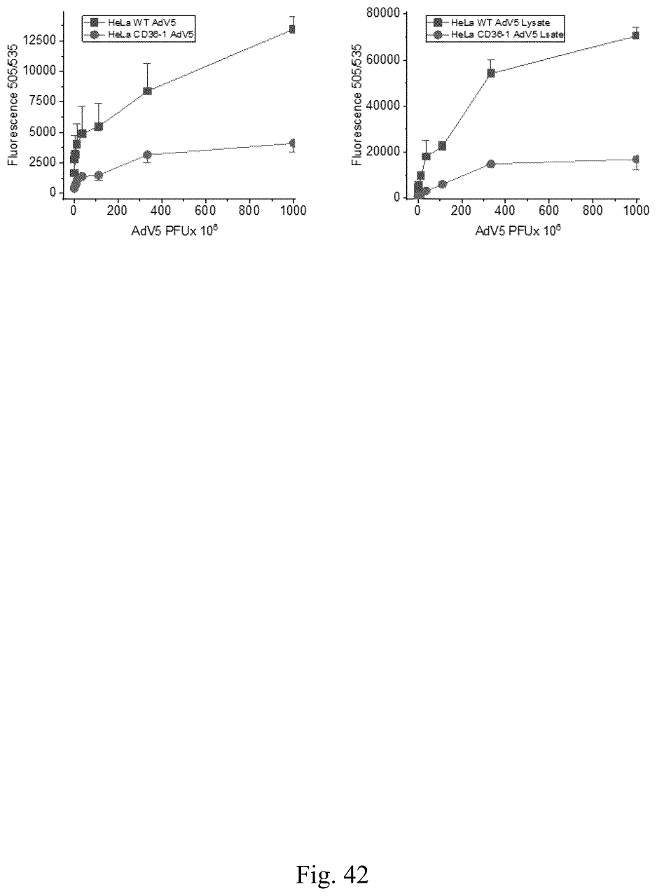

Cell-mediated CD36-dependent AdV5 activity/degradation was measured in vitro utilizing an AdV5-GFP activity degradation assay (FIG. 41). Under conditions (using media absent serum or lipoprotein particles) as used in FIG. 38 where CD36-HeLa demonstrated much higher AdV-Cy2 uptake when compared to WT-HeLa, the cytosol of these cells was isolated and used to transfect HeLa WT cells. The cytosol from overexpressing cells exhibited a much lower transduction efficiency compared to WT cells. This suggests that during the transfection process, the cytosolic AdV5-GFP from the overexpressing cells is processed, degraded, sequestered or modified such that the subsequent transduction efficiency is reduced compared to WT cells (FIG. 42). If this effect results from AdV5 degradation, such would demonstrate that CD36 mediates AdV5-vector degradation both in vitro as well as in vivo.

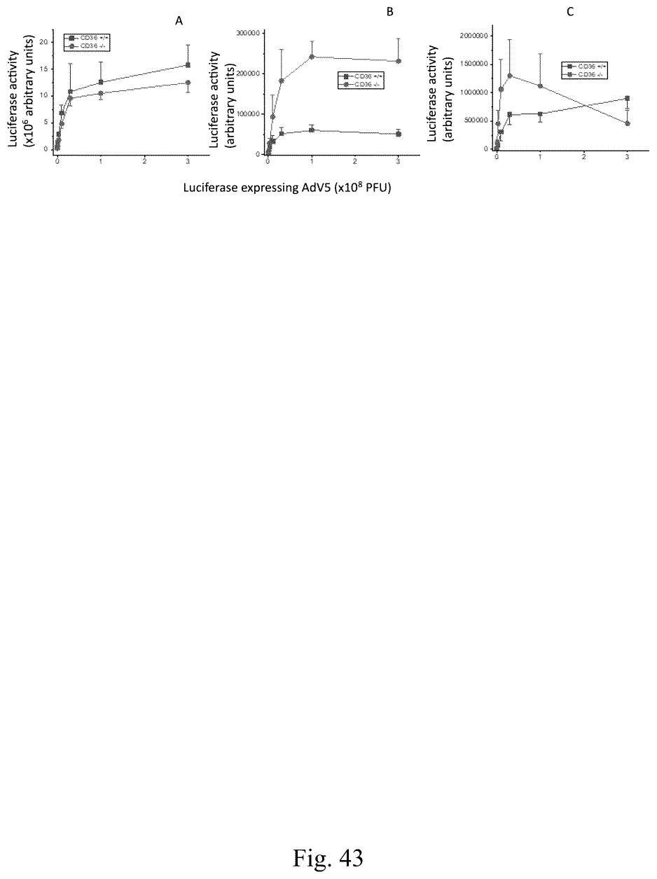

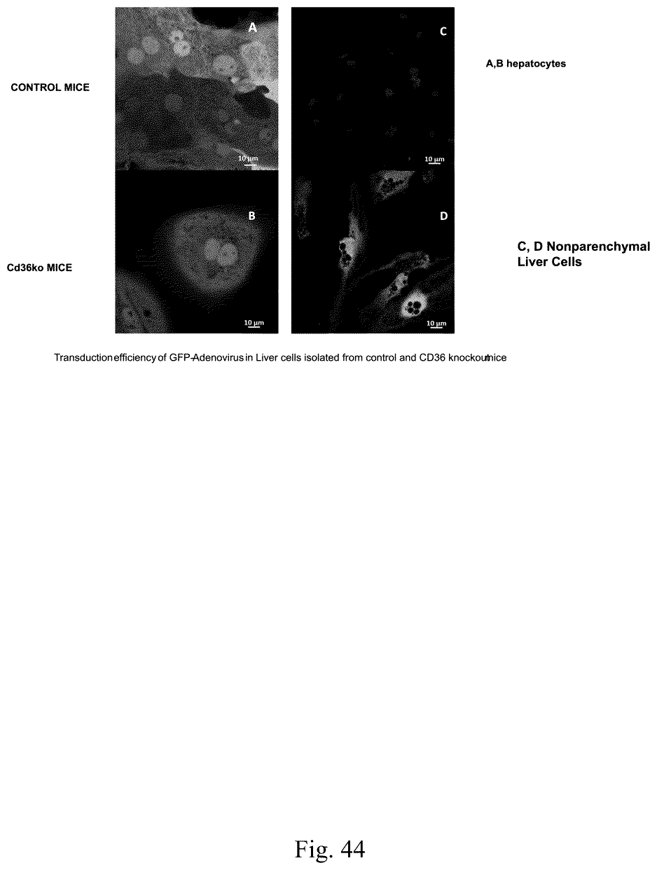

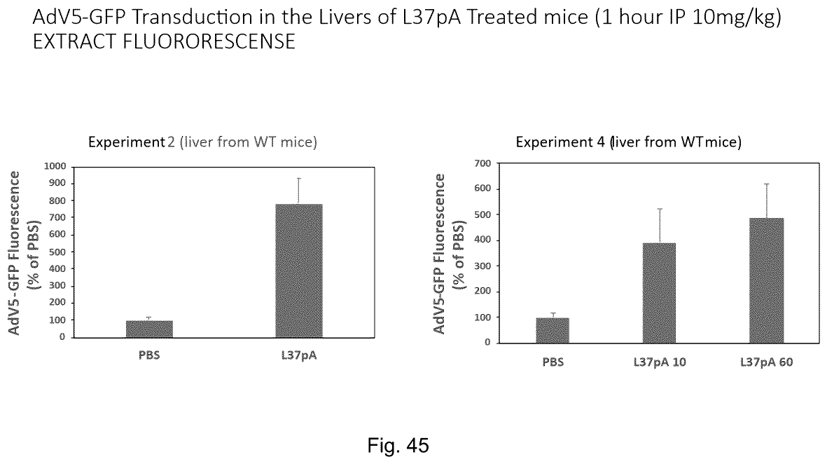

Liver Sinusoidal Endothelial cells (LSEC) from CD36 KO mice had greater gene transduction by AdV5-Luiferase when compared to WT LSEC, indicating a lack of AdV5 degradation in LSECs when CD36 expression was absent (FIG. 43). Similar results are presented in FIG. 44, where GFP transduction was evaluated in cultures of liver hepatocytes and LSECs isolated from CD36KO and WT mice. Hepatocytes from both were transfectable while only CD36KO LSEC expressed GFP (FIG. 44), suggesting that AdV5 degradation mediated by CD36 is absent, allowing GFP transduction. Intraperitoneal injection of the L37 pA peptide increases AdV5-GFP transduction in mouse liver (FIG. 45) compared to PBS with an apparent increased effect when increasing the dose from 10 mg/kg to 60 mg/kg.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of viral vector-mediated gene transfer to an animal or to cells ex vivo, the method comprising (a) administering a viral vector comprising a desired transgene to an animal or to cells ex vivo and (b) at least one of the following:
(i) administering to the animal or to cells ex vivo an agent that antagonizes a member of the class B scavenger receptor (SR-B) family of proteins or down-regulates the expression/activity of an SR-B or
(ii) administering to the animal or to cells ex vivo an agent that up-regulates the expression/activity of an SR-B.

2. The method according to claim 1, wherein the viral vector is derived from an adenovirus (AdV), an adeno-associated virus (AAV), Murine Hepatitis Virus (MHV), or a lentivirus.

3. The method according to claim 1, wherein the up-regulation or down-regulation of the expression/activity of an SR-B comprises regulating the expression/activity of an SR-B involved in viral vector degradation.

4. The method according to claim 1, wherein the viral vector is derived from AdV5, AdV26, AAV 2/8/9, or lentiviruses.

5. The method according to claim 1, wherein the agent that (a) antagonizes an SR-B or down-regulates the expression/activity of an SR-B or (b) up-regulates the expression/activity of an SR-B, comprises a synthetic amphipathic helical peptide (SAHP) or an antibody targeting the SR-B.

6. The method according to claim 5, wherein the agent comprises a SAHP having an amino acid sequence consisting of SEQ ID NOs: 1-61.

7. The method according to claim 5, wherein the agent comprises a SAHP selected from L37 pA, ELK-B, ELKB-2, and ELKB-18A.

8. The method according to claim 1, wherein the agent that down-regulates the expression/activity of an SR-B comprises an interfering RNA molecule (shRNA) targeting the mRNA sequence encoding the SR-B.

9. The method according to claim 8, wherein the shRNA molecule comprises a sequence selected from the group of sequences consisting of SEQ ID Nos 62-73.

10. The method according to claim 1, wherein the agent that up-regulates or down-regulates the expression/activity of an SR-B comprises a small molecule.

11. The method according to claim 1, wherein (a) precedes (b).

12. The method according to claim 1, wherein (b) precedes (a)

13. The method according to claim 1, wherein (a) and (b) are concomitant.

14. The method according to claim 1, wherein the animal is human.

15. In a method comprising viral vector-mediated gene transfer to an animal or to cells ex vivo, the improvement comprising
(i) administering to the animal or to cells ex vivo an agent that antagonizes a member of the class B scavenger receptor (SR-B) family of proteins or down-regulates the expression/activity of an SR-B or
(ii) administering to the animal or to cells ex vivo an agent that up-regulates the expression/activity of an SR-B.

16. The method according to claim 15, wherein the animal is human.

17. A method of promoting viral clearance in an animal, the method comprising administering to an animal having or at risk of a viral infection an active principal that up-regulates the expression/activity of one or more SR-Bs.

18. The method of claim 17, wherein the active principal is an asymmetric SAHP that comprises a first virus specific binding domain and a second SR-B specific binding domain, optionally connected via a spacer.

19. The method according to claim 17, wherein the animal is human.


SEQUENCE LISTING (73 sequences)

SEQ ID NO: 1 - protein, 18 aa, synthetic construct
DWLKAFYDKVAEKLKEAF

SEQ ID NO: 2 - protein, 18 aa, synthetic construct
EKLKELLEKLLEKLKELL

SEQ ID NO: 3 - protein, 18 aa, synthetic construct
ERLRELLERLLERLRELL

SEQ ID NO: 4 - protein, 37 aa, synthetic construct
DWLKAFYDKVAEKLKEAFPDWLKAFYDKVAEKLKEAF

SEQ ID NO: 5 - protein, 37 aa, synthetic construct
DWLKAFYDKVAEKLKEAFPDWAKAAYDKAAEKAKEAA

SEQ ID NO: 6 - protein, 37 aa, synthetic construct
DHLKAFYDKVACKLKEAFPNWAKAAYDKAAEKAKEAA

SEQ ID NO: 7 - protein, 37 aa, synthetic construct
DWLKAFYDKVAEKLKEAFPDHAKAAYDKAACKAKEAA

SEQ ID NO: 8 - protein, 37 aa, synthetic construct
EKLKELLEKLLEKLKELLPEKLKELLEKLLEKLKELL

SEQ ID NO: 9 - protein, 37 aa, synthetic construct
EKLLELLKKLLELLKKLLPEKLLELLKKLLELLKKLL

SEQ ID NO: 10 - protein, 37 aa, synthetic construct
EKLKELLEKLLELLKKLLPEKLKELLEKLLELLKKLL

SEQ ID NO: 11 - protein, 37 aa, synthetic construct
EELKEKLEELKEKLEEKLPEELKEKLEELKEKLEEKL

SEQ ID NO: 12 - protein, 37 aa, synthetic construct
EELKAKLEELKAKLEEKLPEELKAKLEELKAKLEEKL

SEQ ID NO: 13 - protein, 37 aa, synthetic construct
EKLKELLEKLKAKLEELLPEKLKELLEKLKAKLEELL

SEQ ID NO: 14 - protein, 37 aa, synthetic construct
EKLKAKLEELKAKLEELLPEKLKAKLEELKAKLEELL

SEQ ID NO: 15 - protein, 37 aa, synthetic construct
EKLKALLEKLLAKLKELLPEKLKALLEKLLAKLKELL

SEQ ID NO: 16 - protein, 37 aa, synthetic construct
EKLKELLEKLLAKLKELLPEKLKELLEKLLAKLKELL

SEQ ID NO: 17 - protein, 37 aa, synthetic construct
EWLKELLEKLLEKLKELLPEWLKELLEKLLEKLKELL

SEQ ID NO: 18 - protein, 37 aa, synthetic construct
EKFKELLEKFLEKFKELLPEKFKELLEKFLEKFKELL

SEQ ID NO: 19 - protein, 37 aa, synthetic construct
EKFKELLEKLLEKLKELLPEKFKELLEKLLEKLKELL

SEQ ID NO: 20 - protein, 37 aa, synthetic construct
EELKELLKELLKKLEKLLPEELKELLKELLKKLEKLL

SEQ ID NO: 21 - protein, 37 aa, synthetic construct
EELKKLLEELLKKLKELLPEELKKLLEELLKKLKELL

SEQ ID NO: 22 - protein, 37 aa, synthetic construct
EKLKELLEKLLEKLKELLAEKLKELLEKLLEKLKELL

SEQ ID NO: 23 - protein, 38 aa, synthetic construct
EKLKELLEKLLEKLKELLAAEKLKELLEKLLEKLKELL

SEQ ID NO: 24 - protein, 37 aa, synthetic construct
DWLKAFYDKVACKLKEAFPDWAKAAYNKAAEKAKEAA

SEQ ID NO: 25 - protein, 37 aa, synthetic construct
DHLKAFYDKVAEKLKEAFPDWAKAAYDKAAEKAKEAA

SEQ ID NO: 26 - protein, 37 aa, synthetic construct
EKLKAKLEELKAKLEELLPEKAKAALEEAKAKAEELA

SEQ ID NO: 27 - protein, 37 aa, synthetic construct
EKLKAKLEELKAKLEELLPEHAKAALEEAKCKAEELA

SEQ ID NO: 28 - protein, 37 aa, synthetic construct
ERLLELLRRLLELLRRLLPERLLELLRRLLELLRRLL

SEQ ID NO: 29 - protein, 37 aa, synthetic construct
ERLLELLRRLLELLRRLLPDWLKAFYDKVAEKLKEAF

SEQ ID NO: 30 - protein, 37 aa, synthetic construct
EKLLELLKKLLELLKKLLPDWLKAFYDKVAEKLKEAF

SEQ ID NO: 31 - protein, 37 aa, synthetic construct; identical to SEQ ID NO: 30

SEQ ID NO: 32 - protein, 18 aa, synthetic construct
DWLKAFYDKVAEKLKEAF

SEQ ID NO: 33 - protein, 37 aa, synthetic construct; identical to SEQ ID NO: 4

SEQ ID NO: 34 - protein, 18 aa, synthetic construct
EKLKELLEKLLEKLKELL

SEQ ID NO: 35 - protein, 18 aa, synthetic construct
ERLLELLRRLLELLRRLL

SEQ ID NO: 36 - protein, 37 aa, synthetic construct; identical to SEQ ID NO: 9

SEQ ID NO: 37 - protein, 37 aa, synthetic construct; identical to SEQ ID NO: 10

SEQ ID NO: 38 - protein, 37 aa, synthetic construct; identical to SEQ ID NO: 11

SEQ ID NO: 39 - protein, 37 aa, synthetic construct; identical to SEQ ID NO: 12

SEQ ID NO: 40 - protein, 37 aa, synthetic construct; identical to SEQ ID NO: 13

SEQ ID NO: 41 - protein, 37 aa, synthetic construct; identical to SEQ ID NO: 14

SEQ ID NO: 42 - protein, 37 aa, synthetic construct; identical to SEQ ID NO: 15

SEQ ID NO: 43 - protein, 37 aa, synthetic construct; identical to SEQ ID NO: 16

SEQ ID NO: 44 - protein, 37 aa, synthetic construct; identical to SEQ ID NO: 17

SEQ ID NO: 45 - protein, 37 aa, synthetic construct; identical to SEQ ID NO: 18

SEQ ID NO: 46 - protein, 37 aa, synthetic construct; identical to SEQ ID NO: 19

SEQ ID NO: 47 - protein, 37 aa, synthetic construct; identical to SEQ ID NO: 20

SEQ ID NO: 48 - protein, 37 aa, synthetic construct; identical to SEQ ID NO: 21

SEQ ID NO: 49 - protein, 37 aa, synthetic construct; identical to SEQ ID NO: 22

SEQ ID NO: 50 - protein, 38 aa, synthetic construct; identical to SEQ ID NO: 23

SEQ ID NO: 51 - protein, 37 aa, synthetic construct; identical to SEQ ID NO: 24

SEQ ID NO: 52 - protein, 37 aa, synthetic construct; identical to SEQ ID NO: 8

SEQ ID NO: 53 - protein, 36 aa, synthetic construct
ERLRELLERLLERLRELPERLRELLERLLERLRELL

SEQ ID NO: 54 - protein, 37 aa, synthetic construct; identical to SEQ ID NO: 25

SEQ ID NO: 55 - protein, 37 aa, synthetic construct; identical to SEQ ID NO: 26

SEQ ID NO: 56 - protein, 37 aa, synthetic construct; identical to SEQ ID NO: 27

SEQ ID NO: 57 - protein, 37 aa, synthetic construct; identical to SEQ ID NO: 5

SEQ ID NO: 58 - protein, 37 aa, synthetic construct; identical to SEQ ID NO: 6

SEQ ID NO: 59 - protein, 37 aa, synthetic construct; identical to SEQ ID NO: 7

SEQ ID NO: 60 - protein, 37 aa, synthetic construct; identical to SEQ ID NO: 8

SEQ ID NO: 61 - protein, 37 aa, synthetic construct; identical to SEQ ID NO: 28

SEQ ID NO: 62 - RNA, 19 nt, Homo sapiens
  source 1..19: mol_type = genomic RNA
  modified_base 8: mod_base = OTHER; note = uracil
  modified_base 10: mod_base = OTHER; note = uracil
  modified_base 16: mod_base = OTHER; note = uracil
cgacacatataaaggtaaa

SEQ ID NO: 63 - RNA, 19 nt, Homo sapiens
  source 1..19: mol_type = genomic RNA
  modified_base 8: mod_base = OTHER; note = URACIL
  modified_base 10: mod_base = OTHER; note = uracil
  modified_base 12: mod_base = OTHER; note = uracil
  modified_base 16..18: mod_base = OTHER; note = uracil
ggagacctgtgtacatttc

SEQ ID NO: 64 - RNA, 19 nt, Homo sapiens
  source 1..19: mol_type = genomic RNA
  modified_base 3: mod_base = OTHER; note = uracil
  modified_base 10: mod_base = OTHER; note = uracil
  modified_base 13: mod_base = OTHER; note = uracil
  modified_base 17: mod_base = OTHER; note = uracil
  modified_base 19: mod_base = OTHER; note = uracil
cataggacatactggatat

SEQ ID NO: 65 - RNA, 19 nt, Homo sapiens
  source 1..19: mol_type = genomic RNA
  modified_base 2: mod_base = OTHER; note = uracil
  modified_base 4..6: mod_base = OTHER; note = uracil
  modified_base 10: mod_base = OTHER; note = uracil
gtatttgaatccgacgtta

SEQ ID NO: 66 - RNA, 19 nt, Homo sapiens
  source 1..19: mol_type = genomic RNA
  modified_base 8..9: mod_base = OTHER; note = uracil
  modified_base 14..15: mod_base = OTHER; note = uracil
  modified_base 17..19: mod_base = OTHER; note = uracil
ggacaagttcggattattt

SEQ ID NO: 67 - RNA, 19 nt, Homo sapiens
  source 1..19: mol_type = genomic RNA
  modified_base 5: mod_base = OTHER; note = uracil
  modified_base 8: mod_base = OTHER; note = uracil
  modified_base 10: mod_base = OTHER; note = uracil
  modified_base 12: mod_base = OTHER; note = uracil
  modified_base 18: mod_base = OTHER; note = uracil
gaactgctctgtgaaactg

SEQ ID NO: 68 - RNA, 19 nt, Homo sapiens
  source 1..19: mol_type = genomic RNA
  modified_base 1: mod_base = OTHER; note = uracil
  modified_base 5: mod_base = OTHER; note = uracil
  modified_base 10: mod_base = OTHER; note = uracil
  modified_base 17: mod_base = OTHER; note = uracil
tgactggcctgcaccctaa

SEQ ID NO: 69 - RNA, 19 nt, Homo sapiens
  source 1..19: mol_type = genomic RNA
  modified_base 9: mod_base = OTHER; note = uracil
  modified_base 17..18: mod_base = OTHER; note = uracil
ggacaaactgggaagattg

SEQ ID NO: 70 - RNA, 19 nt, Homo sapiens
  source 1..19: mol_type = genomic RNA
  modified_base 3: mod_base = OTHER; note = uracil
  modified_base 8: mod_base = OTHER; note = uracil
  modified_base 10..11: mod_base = OTHER; note = uracil
  modified_base 17..18: mod_base = OTHER; note = uracil
gatgaaatcttgtccctta

SEQ ID NO: 71 - RNA, 19 nt, Homo sapiens
  source 1..19: mol_type = genomic RNA
  modified_base 1: mod_base = OTHER; note = uracil
  modified_base 5..6: mod_base = OTHER; note = uracil
  modified_base 10: mod_base = OTHER; note = uracil
  modified_base 13: mod_base = OTHER; note = uracil
  modified_base 17: mod_base = OTHER; note = uracil
tcacttgactggtggataa

SEQ ID NO: 72 - RNA, 19 nt, Homo sapiens
  source 1..19: mol_type = genomic RNA
  modified_base 1: mod_base = OTHER; note = uracil
  modified_base 9..10: mod_base = OTHER; note = uracil
  modified_base 17..18: mod_base = OTHER; note = uracil
tcacacagttgacgaattg

SEQ ID NO: 73 - RNA, 19 nt, Homo sapiens
  source 1..19: mol_type = genomic RNA
  modified_base 6: mod_base = OTHER; note = uracil
  modified_base 10: mod_base = OTHER; note = uracil
  modified_base 18: mod_base = OTHER; note = uracil
gccaatacgtcagacaatg